(19) United States
(12) Patent Application Publication
(10) Pub. No.: US 2010/0292292 A1
(43) Pub. Date: Nov. 18, 2010

(54) Treatment of Neurodegenerative Diseases Using Indatraline Analogs

(75) Inventors: Peter T. Lansbury, JR., Brookline, MA (US); Craig J. Justman, Cambridge, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: LINK MEDICINE CORPORATION, CAMBRIDGE, MA (US)

(21) Appl. No.: 12/678,225

(22) PCT Filed: Sep. 12, 2008
(86) PCT No.: PCT/US08/76172

§ 371 Date: Jun. 24, 2010

Related U.S. Application Data
(60) Provisional application No. 60/972,053, filed on Sep. 13, 2007.

(51) Int. Cl. A61K 31/135; C07C 211/33; A61K 31/405; C07D 209/14; A61P 25/00; A61P 25/28

(52) U.S. Cl. 514/415; 564/308; 514/647; 548/503

(57) ABSTRACT

Methods and compositions useful in the treatment or prevention of synucleinopathies, such as Parkinson's disease, diffuse Lewy body disease, and multiple system atrophy, or other neurodegenerative diseases (e.g., amyotrophic lateral sclerosis, Huntington's disease, and Alzheimer's disease) are provided. The treatment including administering to a subject an indatraline derivative that inhibits the aggregation of α-synuclein.

### RELATED APPLICATIONS

The present invention claims priority under 35 U.S.C. §119(e) to U.S. provisional patent application, U.S. Ser. No. 60/972,053, filed Sep. 13, 2007, which is incorporated herein by reference.

### FIELD OF THE INVENTION

The present invention relates to the treatment of neurodegenerative diseases, particularly synucleinopathies, such as Parkinson's disease (PD), diffuse Lewy body disease (DLBD), multiple system atrophy (MSA), and various neuronal brain iron accumulation disorders including pantothenate kinase-associated neurodegeneration, using an α-synuclein aggregation inhibitor based on the structure of indatraline.

### BACKGROUND OF THE INVENTION

Synucleinopathies are a diverse group of neurodegenerative disorders that share common pathologic lesions including abnormal aggregates of insoluble α-synuclein protein in selectively vulnerable populations of neurons and glia. Certain evidence links the formation of filamentous aggregates to the onset and progression of clinical symptoms and the degeneration of affected brain regions in neurodegenerative disorders including Parkinson's disease (PD), diffuse Lewy body disease (DLBD), multiple system atrophy (MSA), and disorders of brain iron concentration including pantothenate kinase-associated neurodegeneration (e.g., PANK1).

Lewy bodies, comprising fibrillar α-synuclein, are characteristic of the Parkinson's disease brain. The hypothesis that α-synuclein aggregation drives disease progression is supported by pathological and genetic studies: (1) the progression of Lewy body pathology in the brain follows closely the typical progression of PD symptoms, from autonomic problems (brainstem involvement) to movement disorders (midbrain) to cognitive problems (cortex) (Braak et al., J. Neural. Sci.; 248:255-8, 2006); (2) rare, autosomal dominant forms of PD involve triplication or duplication of the gene and comparable overexpression of the wild-type protein (Singleton et al., Science, 302:841, 2003; Chartier-Harlin et al., Lancet, 364:1167-9, 2004); (3) common (ca. 3% of sporadic PD) polymorphisms in the promoter region affect α-synuclein expression and PD risk (Pals et al., Ann. Neurol., 56:591-5, 2004; Chiba-Falek et al., Hum. Mol. Genet., 10:3101-9, 2001); and (4) haplotypes that are also likely to affect protein expression level segregate with PD risk (Tan et al., Neurology, 62:128-31, 2004).

The current treatment options for these debilitating neurodegenerative diseases include symptomatic medications such as carbidopa-levodopa, anticholinergics, and monoamine oxidase inhibitors, with widely variable benefit. Even for the best responders, i.e., patients with idiopathic Parkinson's Disease, an initial good response to levodopa is typically overshadowed by drug-induced complications such as motor fluctuations and debilitating dyskinesia, following the first five to seven years of therapy. For the other disorders, the current medications offer marginal symptomatic benefit. Given the severe debilitating nature of these disorders and their prevalence, there is a clear need in the art for novel approaches towards treating and managing these diseases.

### SUMMARY OF THE INVENTION

The present invention provides novel compounds that inhibit the aggregation of α-synuclein. These compounds are based on the structure of indatraline, a monoamine oxidase inhibitor that has also been found to inhibit α-synuclein aggregation.

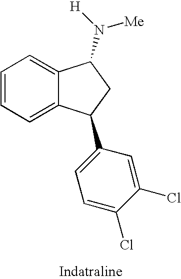

These compounds provide novel therapeutic approaches to the treatment of synucleinopathies, such as Parkinson's disease (PD), diffuse Lewy body disease (DLBD), multiple system atrophy (MSA), and disorders of brain iron concentration including pantothenate kinase-associated neurodegeneration (e.g., PANK1). Other neurodegenerative diseases where abnormal synuclein metabolism or accumulation has been implicated such as amyotrophic lateral sclerosis (ALS), Huntington's Disease (HD), and Alzheimer's Disease (AD) (including Alzheimer's Disease with Lewy Bodies) may also be treated with the inventive compounds. The diseases may also be prevented using the inventive compounds. The invention also provides methods of preparing the inventive compounds, methods of using the inventive compounds to treat and prevent neurodegenerative diseases in subject, and pharmaceutical compositions of the inventive compounds.

In one aspect, the present invention provides compounds of formula I:

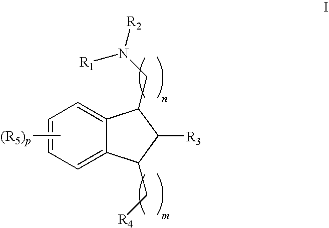

wherein

n is an integer between 0 and 6, inclusive;

m is an integer between 0 and 6, inclusive;

p is an integer between 0 and 4, inclusive;

R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{A}; —CO₂R_{A}; —C(═O)N(R_{A})₂; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; —C(═O)N(R_{B})₂; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is substituted or unsubstituted, branched or unbranched aryl; or substituted or unsubstituted, branched or unbranched heteroaryl;

R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable forms thereof. In certain embodiments, the substituents off the core ring system may be cis (i.e., both on the same face). In other embodiments, the substituents off the core ring system may be trans (i.e., each on a different face).

According to the invention, compounds of formula I which are based on the structure of indatraline have been shown to prevent the aggregation of α-synuclein. In certain embodiments, the compounds have been shown to bind α-synuclein and increase the rate of α-synuclein aggregation in 1,1,1,3,3,3-hexafluoroisopropanol (HFIP). See Example 10 and FIGS. 1-17. Increased rates of α-synuclein aggregation in this artificial system have been found to be indicative of reducing α-synuclein aggregation in vivo. Indatraline derivatives of formula I were also found to reduce α-synuclein aggregation in an aqueous buffered solution relevant to physiological conditions. See Example 11 and FIGS. 18-25. In vivo indatraline has been found to suppress α-synuclein-induced retinal degeneration in transgenic Drosophila (see FIG. 26A) and to decrease α-synuclein neurotoxicity in dopaminergic neurons (see FIG. 26B).

In another aspect, the invention provides methods of inhibiting α-synuclein aggregation by contacting α-synuclein protein with an effective amount of a compound of formula I to reduce the aggregation of α-synuclein. The method may be carried out in vitro or in vivo. In another aspect, the invention provides methods of treating cells expressing α-synuclein by contacting cells with an effective amount of a compound of formula I to reduce the aggregation of α-synuclein. The treatment of cells with the inventive compounds may be carried out in vivo or in vitro.

In another aspect, the invention provides methods of treating a synucleinopathic subject with a therapeutically effective amount of compound of formula I. In certain embodiments, the synucleinopathic subject has a synucleinopathy or disease associated with abnormal synuclein accumulation or metabolism. In certain embodiments, the subject is a human. In certain embodiments, the subject suffers from Parkinson's disease (PD), diffuse Lewy body disease (DLBD), multiple system atrophy (MSA), or a disorder of brain iron concentration including pantothenate kinase-associated neurodegeneration (e.g., PANK1). In certain embodiments, the subject suffers from Alzheimer's disease (AD). In certain embodiments, the subject suffers from Huntington's disease (HD).

In yet another aspect, the invention provides methods of inhibiting α-synuclein aggregation using an effective amount of a combination of a farnesyl transferase inhibitor and an α-synuclein aggregation inhibitor of formula I to reduce the aggregation of α-synuclein. This aspect of the invention in part stems from the recognition that when both these agents are administered to a subject there is an additive or synergistic effect between the two agents. That is, in certain embodiments, lower doses of at least one of these agents can be used than when either agents is administered individually. The inventive combination therapy may be used in vivo or in vitro to inhibit α-synuclein aggregation. For example, the combination may be administered to a subject (e.g., mouse, rat, human), or cells in cell culture may be contacted with a combination of an inventive compound of formula I and a farnesyl transferase inhibitor.

The invention also provides pharmaceutical compositions comprising a therapeutically effective amount of a compound of formula I. The composition may optionally include a pharmaceutically acceptable excipient. In certain embodiments, the therapeutically effective amount of the compound of formula I that inhibits the aggregation of α-synuclein or a pharmaceutically acceptable form thereof comprises about 10 ng/kg of body weight to about 1000 mg/kg of body weight at a frequency of administration from once a day to once a month. In certain embodiments, the composition may further comprise a farnesyl transferase inhibitor. In certain embodiments, the therapeutically effective amount of each agent or pharmaceutically acceptable form thereof comprises about 10 ng/kg of body weight to about 1000 mg/kg of body weight at a frequency of administration from once a day to once a month. The amount of one or both of the agents may be lower than when either agent is administered alone. In certain embodiments, the composition may also include other pharmaceutical agents for treating synucleinopathic subjects or subjects with neurodegenerative diseases. Such agents may include drugs for treating the symptoms of the disease rather than the disease itself.

In another aspect, the invention provides kits comprising pharmaceutical compositions of a compound of formula I. The kit optionally includes instructions for prescribing the medication. In certain embodiments, the kit includes multiple doses. The kit may include sufficient quantities of each component to treat a subject for a week, two weeks, three weeks, four weeks, or multiple months. In certain embodiments, such kits include the inventive combination of a farnesyl transferase inhibitor and a compound of formula I. The agents may be packaged separately or together.

This application refers to various issued patents, published patent applications, journal articles, and other publications, all of which are incorporated herein by reference.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomeric mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidene ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonoethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulae of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of synucleinopathies or other neurodegenerative diseases. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6, or 7 carbons in the ring structure.

Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

As used herein, the term “halogen” designates —F, —Cl, —Br or —I; the term “sulfhydryl” or “thiol” means —SH; and the term “hydroxyl” means —OH.

The term “methyl” refers to the monovalent radical —CH₃, and the term “methoxyl” refers to the monovalent radical —CH₂OH.

The term “aralkyl” or “arylalkyl,” as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms “ortho”, “meta”, and “para” apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” or “heteroaryl” refer to 3- to 10-membered ring structures, more preferably 3- to 7-membered rings, whose ring structures include one to four heteroatoms. Heterocycles can also be polycycles. Heterocyclyl groups include, for example, thiophene, benzothiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the definition of each expression, e.g., alkyl, m, n, etc., when it occurs more than once in any structure, is intended to be independent of its definition elsewhere in the same structure.

Definitions of other terms used throughout the specification include:

As used herein and in the appended claims, the singular forms “a”, “an”, and “the” include the plural reference unless the context clearly indicates otherwise. Thus, for example, a reference to “a cell” includes a plurality of such cells.

“Animal”: As used herein, the term “animal” refers to any member of the animal kingdom. In some embodiments, “animal” refers to a human at any stage of development. In some embodiments, “animal” refers to a non-human animal at any stage of development. In some embodiments, animals include, but are not limited to, mammals, birds, reptiles, amphibians, fish, and/or worms. In certain embodiments, the animal is a vertebrate. In certain embodiments, the non-human animal is a mammal (e.g., an ape, a rodent, a mouse, a rat, a rabbit, a monkey, a dog, a cat, a sheep, cattle, a primate, and/or a pig). In some embodiments, an animal may be a transgenic animal, genetically-engineered animal, and/or clone.

“Effective amount”: In general, the “effective amount” of an active agent or combination of agents refers to an amount sufficient to elicit the desired biological response. As will be appreciated by those of ordinary skill in this art, the effective amount of an inventive combination may vary depending on such factors as the desired biological endpoint, the pharmacokinetics of the agents being delivered, the disease being treated, the mode of administration, and the patient. For example, the effective amount of an inventive compound (i.e., α-synuclein aggregation inhibitor) is the amount of the compound that when administered results in reducing α-synuclein aggregation in a subject.

“Pharmaceutically acceptable”: The present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier”: The phrase “pharmaceutically-acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

“Pharmaceutically acceptable salt”: The term “pharmaceutically acceptable salt” as used herein is meant to comprise the therapeutically active non-toxic acid and non-toxic base addition salt forms that the compounds are able to form. The compounds that have basic properties can be converted into their pharmaceutically acceptable acid addition salts by treating the base form with an appropriate acid. Appropriate acids include, for example, inorganic acids such as hydrohalic acids, e.g., hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e., butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. In certain embodiments, the salt is a tartrate salt. The tartrate salt may be either L-tartric acid or D-tartric acid. Both tartric acids are available from Aldrich Chemical Company, Inc. (Milwaukee, Wis.). The salts may be anhydrous or hydrous forms.

The compounds that have acidic properties can be converted into their pharmaceutically acceptable base addition salts by treating the acid form with a suitable organic or inorganic base. Appropriate base salt forms include, for example, the ammonium salts, the alkali and earth alkaline metal salts, e.g., the lithium, sodium, potassium, magnesium, calcium salts and the like, salts with organic bases, e.g., the benzathine, N-methyl-D-glucamine, hydrabamine salts, and salts with amino acids such as, for example, arginine, lysine and the like.

The terms acid or base addition salt also comprise the hydrates and the solvent addition forms that the compounds are able to form. Examples of such forms are, e.g., hydrates, alcoholates, and the like.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19; incorporated herein by reference).

The pharmaceutically acceptable salts of the inventive compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine, and the like. See, for example, Berge et al., supra.

“Small molecule”: As used herein, the term “small molecule” is used to refer to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention.

“Synucleinopathic subject”: As used herein, the term “synucleinopathic subject” or “subject with a synucleinopathy” refers to a subject that is diagnosed with, affected by, or at risk of developing a synucleinopathy (e.g., predisposed, for example genetically predisposed, to developing a synucleinopathy, or for whom biomarkers suggest a pre-clinical state) and/or any neurodegenerative disorder characterized by pathological synuclein aggregations. Several neurodegenerative disorders including Parkinson's disease, diffuse Lewy body disease (DLBD), multiple system atrophy (MSA), and disorders of brain iron concentration including pantothenate kinase-associated neurodegeneration (e.g., PANK1) are collectively grouped as synucleinopathies. In certain particular embodiments, the synucleinopathy is Parkinson's disease. In certain embodiments, the synucleinopathy is diffuse Lewy body disease (DLBD). In certain embodiments, the synucleinopathy is multiple system atrophy. In certain particular embodiments, the synucleinopathy is a disorder of brain iron concentration (e.g., pantothenate kinase-associated neurodegeneration). In certain embodiments, the synucleinopathy may not be a prototypical synucleinopathy as described above but may include such diseases as amyotrophic lateral sclerosis (ALS), Huntington's Disease (HD), and Alzheimer's Disease (AD) (including Alzheimer's Disease with Lewy Bodies).

“Therapeutically effective amount”: The phrase “therapeutically effective amount” as used herein means that amount of a compound or composition which is effective for producing some desired therapeutic effect in a subject at a reasonable benefit/risk ratio applicable to any medical treatment. Accordingly, a therapeutically effective amount prevents, minimizes, slows, or reverses disease progression associated with a synucleinopathy or other neurodegenerative disease. Disease progression can be monitored by clinical observations, laboratory, and/or neuroimaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

“Treatment”: According to the invention, the term “treatment” includes prophylaxis and therapy, and includes managing a subject's symptoms and halting the progression of the disease. Treatment includes preventing, slowing, stopping, or reversing (e.g., curing) the development of a synucleinopathy or other neurodegenerative disease, and/or the onset of certain symptoms associated with a synucleinopathy or other neurodegenerative disease in a subject with, or at risk of developing, a synucleinopathy, a related disorder, or other neurodegenerative disease. For the treatment of a synucleinopathy, the therapy typically includes preventing, slowing, stopping or reversing (e.g., curing) the accumulation and/or aggregation of α-synuclein in a subject with a synucleinopathy. Therapy also includes decreasing the amount of accumulated α-synuclein in a subject with a synucleinopathy or other neurodegenerative disorder.

### BRIEF DESCRIPTION OF THE DRAWING

FIG. 1 shows that indatraline binds to α-synuclein and affects the rate of structure formation in the presence of 1,1,1,3,3,3-hexafluoro-2-propanol (HFIP). A. Monitoring of Thioflavin T fluorescence. B. Monitoring of α-synuclein fluorescence polarization. Solvent alone (solid line), indatraline at 50 μM (open squares), 100 μM (open circles), 200 μM (open triangles), or 400 μM (closed squares).

FIG. 2 shows that LNK-121 binds to α-synuclein and affects the rate of structure formation in the presence of HFIP. A. Monitoring of Thioflavin T fluorescence. B. Monitoring of α-synuclein fluorescence polarization. Solvent alone (solid line), LNK-121 at 50 μM (open squares), 100 μM (open circles), 200 μM (open triangles), or 400 μM (closed squares).

FIG. 3 shows that both cis-LNK-121 and trans-LNK-121 bind to α-synuclein and affect the rate of structure formation in the presence of HFIP. Monitoring of Thioflavin T fluorescence in the presence of A. cis-LNK-121 B. trans-LNK-121. Solvent alone (solid line), LNK-121 at 50 μM (open squares), 100 μM (open circles), or 200 μM (open triangles).

FIG. 4 shows that LNK-122 binds to α-synuclein and affects the rate of structure formation in the presence of HFIP. A. Monitoring of Thioflavin T fluorescence. B. Monitoring of α-synuclein fluorescence polarization. Solvent alone (solid line), LNK-122 at 200 μM (open triangles), or 400 μM (closed squares).

FIG. 5 shows that LNK-123 binds to α-synuclein and affects the rate of structure formation in the presence of HFIP. A. Monitoring of Thioflavin T fluorescence. B. Monitoring of α-synuclein fluorescence polarization. Solvent alone (solid line), LNK-123 at 50 μM (open squares), 100 μM (open circles), 200 μM (open triangles), or 400 μM (closed squares).

FIG. 6 shows that both cis-LNK-123 and trans-LNK-123 bind to α-synuclein and affect the rate of structure formation in the presence of HFIP. Monitoring of Thioflavin T fluorescence in the presence of A. cis-LNK-123 and B. trans-LNK-123. Solvent alone (solid line), LNK-123 at 100 μM (open squares), 200 μM (open circles), 300 μM (open triangles), or 400 μM (closed squares).

FIG. 7 shows that LNK-124 binds to α-synuclein and affects the rate of structure formation in the presence of HFIP. A. Monitoring of Thioflavin T fluorescence. B. Monitoring of α-synuclein fluorescence polarization. Solvent alone (solid line), LNK-124 at 200 μM (open triangles) or 400 μM (closed squares).

FIG. 8 shows that LNK-125 binds to α-synuclein and affects the rate of structure formation in the presence of HFIP. A. Monitoring of Thioflavin T fluorescence. B. Monitoring of α-synuclein fluorescence polarization. Solvent alone (solid line), LNK-125 at 100 μM (open circles), 200 μM (open triangles), or 400 μM (closed squares).

FIG. 9 shows that LNK-126 binds to α-synuclein and affects the rate of structure formation in the presence of HFIP. Monitoring of Thioflavin T fluorescence. Solvent alone (solid line) or LNK-125 at 400 μM (closed squares).

FIG. 10 shows that LNK-130 binds to α-synuclein and affects the rate of structure formation in the presence of HFIP. A. Monitoring of Thioflavin T fluorescence. B. Monitoring of α-synuclein fluorescence polarization. Solvent alone (solid line), LNK-130 at 50 μM (open squares), 100 μM (open circles), 200 μM (open triangles), or 400 μM (closed squares).

FIG. 11 shows that both cis-LNK-1110 and trans-LNK-1110 bind to α-synuclein and affect the rate of structure formation in the presence of HFIP. Monitoring of Thioflavin T fluorescence in the presence of A. cis-LNK-1110 and B. trans-LNK-1110. Solvent alone (solid line), LNK-1110 at 100 μM (open squares), 200 μM (open circles), 300 μM (open triangles), or 400 μM (closed squares).

FIG. 12 shows that both cis-LNK-1111 and trans-LNK-1111 bind to α-synuclein and affect the rate of structure formation in the presence of HFIP. Monitoring of Thioflavin T fluorescence in the presence of A. cis-LNK-1111 and B. trans-LNK-1111. Solvent alone (solid line), LNK-1111 at 100 μM (open squares), 200 μM (open circles), or 400 μM (closed squares).

FIG. 13 shows that both cis-LNK-1114 and trans-LNK-1114 bind to α-synuclein and affect the rate of structure formation in the presence of HFIP. Monitoring of Thioflavin T fluorescence in the presence of A. cis-LNK-1114 and B. trans-LNK-1114. Solvent alone (solid line), LNK-1114 at 100 μM (open squares), 200 μM (open circles), 300 μM (open triangles), or 400 μM (closed squares).

FIG. 14 shows that both cis-LNK-1115 and trans-LNK-1115 bind to α-synuclein and affect the rate of structure formation in the presence of HFIP. Monitoring of Thioflavin T fluorescence in the presence of A. cis-LNK-1115 B. trans-LNK-1115. Solvent alone (solid line), LNK-1115 at 100 μM (open squares), 200 μM (open circles), 300 μM (open triangles), or 400 μM (closed squares).

FIG. 15 shows that both cis-LNK-1521 and trans-LNK-1521 bind to α-synuclein and affect the rate of structure formation in the presence of HFIP. Monitoring of Thioflavin T fluorescence in the presence of A. cis-LNK-1521 and B. trans-LNK-1521. Solvent alone (solid line), LNK-1521 at 50 μM (open squares), 100 μM (open circles), 200 μM (open triangles), or 400 μM (closed squares).

FIG. 16 shows that both cis-LNK-1800 and trans-LNK-1800 bind to α-synuclein and affect the rate of structure formation in the presence of HFIP. Monitoring of Thioflavin T fluorescence in the presence of A. cis-LNK-1800 and B. trans-LNK-1800. Solvent alone (solid line), LNK-1800 at 50 μM (open squares), 100 μM (open circles), 200 μM (open triangles), or 400 μM (closed squares).

FIG. 17 shows that both enantiomers of cis-LNK-1800 bind to α-synuclein and affect the rate of structure formation in the presence of HFIP. Monitoring of Thioflavin T fluorescence in the presence of A. cis-LNK-1800-e1 and B. cis-LNK-1800-e2. Solvent alone (solid line), cis-LNK-1800 enantiomer at 50 μM (open squares), 100 μM (open circles), 200 μM (open triangles), or 400 μM (closed squares).

FIG. 18 shows that indatraline delays aggregation of α-synuclein in a dose-dependent manner. Solvent alone (solid line), indatraline at 1 μM (solid squares), 10 μM (open squares), 50 μM (open circles), or 100 μM (open diamonds).

FIG. 19 shows that both cis-LNK-121 and trans-LNK-121 delay the aggregation of α-synuclein in aqueous solution. Monitoring of Thioflavin T fluorescence in the presence of A. cis-LNK-121 and B. trans-LNK-121. Solvent alone (solid line), LNK-121 at 100 μM (open circles) or 200 μM (closed squares).

FIG. 20 shows that cis-LNK-123 delays the aggregation of α-synuclein in aqueous solution. A. Monitoring of Thioflavin T fluorescence. B. Monitoring of α-synuclein fluorescence polarization. Solvent alone (solid line), cis-LNK-123 at 100 μM (open circles) or 200 μM (closed squares).

FIG. 21 shows that trans-LNK-123 delays the aggregation of α-synuclein in aqueous solution. A. Monitoring of Thioflavin T fluorescence. B. Monitoring of α-synuclein fluorescence polarization. Solvent alone (solid line), trans-LNK-123 at 100 μM (open circles) or 200 μM (closed squares).

FIG. 22 shows that cis-LNK-1800 delays the aggregation of α-synuclein in aqueous solution. A. Monitoring of Thioflavin T fluorescence. B. Monitoring of α-synuclein fluorescence polarization. Solvent alone (solid line), cis-LNK-1800 at 100 μM (open circles) or 200 μM (closed squares).

FIG. 23 shows that trans-LNK-1800 delays the aggregation of α-synuclein in aqueous solution. A. Monitoring of Thioflavin T fluorescence. B. Monitoring of α-synuclein fluorescence polarization. Solvent alone (solid line), trans-LNK-1800 at 100 μM (open circles) or 200 μM (closed squares).

FIG. 24 shows that cis-LNK-1111 delays the aggregation of α-synuclein in aqueous solution. A. Monitoring of Thioflavin T fluorescence. B. Monitoring of α-synuclein fluorescence polarization. Solvent alone (solid line), cis-LNK-1111 at 400 μM (closed squares).

FIG. 25 shows that cis-LNK-1115 delays the aggregation of α-synuclein in aqueous solution. A. Monitoring of Thioflavin T fluorescence. B. Monitoring of α-synuclein fluorescence polarization. Solvent alone (solid line) or cis-LNK-1115 at 400 μM (closed squares).

FIG. 26 shows that indatraline suppresses α-synuclein toxicity in a dose-dependent manner. A. Indatraline decreases α-synuclein-mediated retinal degeneration in transgenic Drosophila. B. Indatraline decreases α-synuclein neurotoxicity in dopaminergic neurons.

### DETAILED DESCRIPTION OF THE INVENTION

Synucleins are small proteins (123 to 143 amino acids) characterized by six or seven copies of an 11-residue imperfect repeat XKTKEGVXXXX (SEQ ID NO: XX) at the N-terminal end, followed by a variable short hydrophobic tail at the C-terminal end. There are three human synuclein proteins termed α, β, and γ, and they are encoded by separate genes which have been mapped to chromosomes 4221.3-q22, 5q23, and 10q23.2-q23.3, respectively. The most recently cloned synuclein protein, synoretin, is closely homologous to γ-synuclein and is predominantly expressed within the retina. α-synuclein, also referred to as the non-amyloid component of senile plaques precursor protein (NACP), SYN1, or synelfin, is a heat-stable, “natively unfolded” protein of poorly defined function. It is predominantly expressed in the central nervous system (CNS) neurons where it is localized to pre-synaptic terminals. Electron microscopy studies have localized α-synuclein in close proximity to synaptic vesicles at axonal termini, suggesting a role for α-synuclein in neurotransmission or synaptic organization, and biochemical analysis has revealed that a small fraction of α-synuclein may be associated with vesicular membranes but most α-synuclein is cytosolic.

Genetic and histopathological evidence supports the idea that α-synuclein is the major component of several proteinaceous inclusions characteristic of specific neurodegenerative diseases. Pathological synuclein aggregations in Parkinson's disease are restricted to the α-synuclein isoforms, as β- and γ-synucleins have not been detected in these inclusions. Lewy bodies, neuronal fibrous cytoplasmic inclusions that are histopathological hallmarks of Parkinson's disease (PD) and diffuse Lewy body disease (DLBD), are strongly labeled with antibodies to α-synuclein. Dystrophic ubiquitin-positive neurites associated with PD pathology, termed Lewy neurites (LN) and CA2/CA3 ubiquitin neurites are also α-synuclein positive. Furthermore, pale bodies, putative precursors of LBs, thread-like structures in the perikarya of slightly swollen neurons and glial silver positive inclusions in the midbrains of patients with LB diseases are also immunoreactive for α-synuclein. α-synuclein is likely the major component of glial cell inclusions (GCIs) and neuronal cytoplasmic inclusions in MSA and some types of brain iron accumulation including PANK1. α-synuclein immunoreactivity is present in some dystrophic neurites in senile plaques in Alzheimer's Disease (AD) and in the cord and cortex in amyotrophic lateral sclerosis (ALS). α-synuclein immunoreactivity is prominent in transgenic and toxin-induced mouse models of PD, AD, ALS, and Huntington's Disease (HD).

Further evidence supports the notion that α-synuclein is the actual building block of the fibrillary components of Lewy bodies, Lewy neurites, and glial cell inclusions. Immunoelectron microscopic studies have demonstrated that these fibrils are intensely labeled with α-synuclein antibodies in situ. Sarcosyl-insoluble α-synuclein filaments with straight and twisted morphologies can also be observed in extracts of DLBD and MSA brains. Moreover, α-synuclein can assemble in vitro into elongated homopolymers with similar widths as sarcosyl-insoluble fibrils or filaments visualized in situ. Polymerization is associated with a concomitant change in secondary structure from a random coil to an anti-parallel β-sheet structure consistent with the thioflavine-S reactivity of these filaments. Furthermore, the PD-association with α-synuclein mutation, A53T, may accelerate this process, as recombinant A53T α-synuclein has a greater propensity to polymerize than wild-type α-synuclein. This mutation also affects the ultrastructure of the polymers; the filaments are slightly wider and are more twisted in appearance, as if assembled from two protofilaments. The A30P mutation may also modestly increase the propensity of α-synuclein to polymerize, but the pathological effects of this mutation also may be related to its reduced binding to vesicles. Interestingly, carboxyl-terminally truncated α-synuclein may be more prone to form filaments than the full-length protein.

The proteosomal degradation of α-synuclein is mediated by parkin and neuronal ubiquitin C-terminal hydrolase (UCH-L1). Parkin is an E3 ligase that ubiquitinylates α-synuclein and thereby tags it for degradation. UCH-L1 acts in normal neuronal tissues to cleave the ubiquitinylated proteins that are products of the proteosomal degradation of the polyubiquitinylated proteins.

It has been discovered that UCH-L1 is farnesylated in vivo. UCH-L1 is associated with the membrane, and this membrane association is mediated by farnesylation. Farnesylated UCH-L1 also stabilizes the accumulation of α-synuclein. The invention in part relates to the prevention or inhibition of UCH-L1 farnesylation which would result in UCH-L1 membrane disassociation and acceleration of the degradation of α-synuclein. Since α-synuclein accumulation is pathogenic in PD, DLBD, and MSA, an increased degradation of α-synuclein and/or inhibition of α-synuclein accumulation ameliorates the toxicity associated with a pathogenic accumulation of α-synuclein.

The modification of a protein by a farnesyl group can have an important effect on function for a number of proteins. Farnesylated proteins typically undergo further C-terminal modification events that include a proteolytic removal of three C-terminal amino acids and carboxymethylation of C-terminal cysteines. These C-terminal modifications facilitate protein-membrane association as well as protein-protein interactions. Farnesylation is catalyzed by a protein farnesyltransferase (FTase), a heterodimeric enzyme that recognizes the CAAX motif present at the C-terminus of the substrate protein. FTase transfers a farnesyl group from farnesyl pyrophosphate and forms a thioether linkage between the farnesyl and the cystine residues in the CAAX motif. A number of inhibitors of FTase have been developed and are known in the art. However, the invention provides novel methods for using certain farnesyl transferase inhibitors to treat subjects having symptoms associated with α-synuclein accumulation.

The term synucleinopathy typically refers to neurological disorders that are characterized by a pathological accumulation and/or aggregation of α-synuclein. This group of disorders includes, but is not limited to, PD, DLBD, MSA, and disorders of brain iron concentration including pantothenate kinase-associated neurodegeneration (e.g., PANK1).

The term synucleinopathic subject encompasses a subject that is affected by, or is at risk of developing a synucleinopathy such as PD, DLBD, MSA, and disorders of brain iron concentration including pantothenate kinase-associated neurodegeneration (e.g., PANK1). These subjects can be readily identified by persons of ordinary skill in the art by symptomatic diagnosis, physical examination, neurologic examination, and/or in some instances in conjunction with genetic screening, brain scans, SPEC, PET imaging, etc.

### Parkinson's Disease

Parkinson's disease (PD) is a neurological disorder characterized by bradykinesia, rigidity, tremor, and postural instability. The pathologic hallmark of PD is loss of neurons in the substantia nigra pars compacta (SNpc) and the appearance of Lewy bodies in remaining neurons. It appears that more than about 50% of the cells in the SNpc need to be lost before motor symptoms appear. Associated symptoms often include small handwriting (micrographia), seborrhea, orthostatic hypotension, urinary difficulties, constipation and other gastrointestinal dysfunction, sleep disorders, depression and other neuropsychiatric phenomena, dementia, and smelling disturbances. Patients with Parkinsonism have greater mortality, about two times compared to the general population without PD. This is attributed to greater frailty and/or reduced mobility.

Diagnosis of PD, at present, is mainly clinical and is based on the clinical findings listed above. Parkinsonism, refers to any combination of two of bradykinesia, rigidity, and/or tremor. PD is the most common cause of parkinsonism. Other causes of parkinsonism are side effects of drugs, mainly the major tranquilizers, such as haloperidol, strokes involving the basal ganglia, and other neurodegenerative disorders, such as Diffuse Lewy Body Disease (DLBD), progressive supranuclear palsy (PSP), frontotemporal dementia (FTD), MSA, and Huntington's disease. The pathological hallmark of PD and DLBD is the Lewy body, an intracytoplasmatic inclusion body typically seen in affected neurons of the substantia nigra and to a variable extent, in the cortex in the former disease, and vice versa in the latter. α-synuclein has been identified as the main component of Lewy bodies in sporadic parkinsonism.

Although parkinsonism can sometimes be attributed to viruses, stroke, or toxins in a few individuals, for the most part, the cause of Parkinson's disease in any particular case is unknown. Environmental influences which may contribute to PD may include drinking well water, farming, and industrial exposure to heavy metals (e.g., iron, zinc, copper, mercury, magnesium and manganese), alkylated phosphates and other pesticides, and orthonal chlorines. Paraquat (a herbicide) has also been associated with increased prevalence of Parkinsonism including PD. Cigarette smoking is associated with a decreased incidence of PD. The current consensus is that PD may either be caused by an uncommon toxin combined with high genetic susceptibility or a common toxin combined with relatively low genetic susceptibility.

A small percentage of subjects that are at risk of developing PD can be identified for example by genetic analysis. There is good evidence for certain genetic factors being associated with PD. Large pedigrees of autosomal dominantly inherited PDs have been reported. For example, three point mutations in the α-synuclein gene (SNCA gene) have been associated with autosomal dominant PD, as duplication and triplication of the wildtype SNCA gene.

### Lewy Body Disease

DLBD is the second most common cause of dementia in older individuals; it affects 7% of the general population older than 65 years and 30% of those aged over 80 years. It is part of a range of clinical presentations that share a pathology based on abnormal aggregation of the synaptic protein α-synuclein. DLBD has many of the clinical and pathological characteristics of the dementia that occur late in the course of Parkinson's disease. A “one year rule” has been proposed to separate DLBD from PD. According to this rule, onset of dementia within 12 months of Parkinsonism qualifies as DLBD, whereas more than 12 months of Parkinsonism before onset of dementia qualifies as Parkinson's Disease with dementia (PDD). The central features of DLBD include progressive cognitive decline of sufficient magnitude to interfere with normal social and occupational function and neuropsychiatric phenomena. Prominent or persistent memory impairment does not necessarily occur in the early stages, but it is evident with progression in most cases. Deficits on tests of attention and of frontal cortical skills and visual spatial ability can be especially prominent.

Core diagnostic features, two of which are essential for diagnosis of probable and one for possible DLBD are fluctuating cognition with pronounced variations in attention and alertness, recurrent visual hallucinations that are typically well-formed and detailed, and spontaneous features of Parkinsonism. In addition, there can be some supportive features, such as repeated falls, syncope, transient loss of consciousness, neuroleptic sensitivity, systematized delusions, hallucinations and other modalities, REM sleep behavior disorder, and depression. Patients with DLBD do better than those with Alzheimer's Disease in tests of verbal memory, but worse on visual performance tests. This profile can be maintained across the range of severity of the disease, but can be harder to recognize in the later stages owing to global difficulties. DLBD typically presents with recurring episodes of confusion on a background of progressive deterioration. Typical patients with DLBD show a combination of cortical and subcortical neuropyschological impairments with substantial attention deficits and prominent frontal subcortical and visual spatial dysfunction. These features of DLBD help differentiate this disorder from Alzheimer's disease.

Rapid eye movement (REM), sleep behavior disorder is a parasomnia manifested by vivid and frightening dreams associated with simple or complex motor behavior during REM sleep. This disorder is frequently associated with the synucleinopathies, DLBD, PD, and MSA, but occurs less often in amyloidopathies and tauopathies. The neuropyschological pattern of impairment in REM sleep behavior disorder/dementia is similar to that reported in DLBD and qualitatively different from that reported in Alzheimer's Disease. Neuropathological studies of REM sleep behavior disorder associated with neurodegenerative disorder have shown Lewy body disease or multiple system atrophy. REM sleep wakefulness disassociations (REM sleep behavior disorder, daytime hypersomnolence, hallucinations, cataplexy) characteristic of narcolepsy can explain several features of DLBD, as well as PD. Sleep disorders could contribute to the fluctuations typical of DLBD, and their treatment can improve fluctuations and quality of life. Subjects at risk of developing DLBD can be identified. Repeated falls, syncope, transient loss of consciousness, and depression are common in older people with cognitive impairment and can serve as a clue to a possible diagnosis of DLBD. By contrast, narcoleptic sensitivity in REM sleep behavior disorder can be highly predictive of DLBD. Their detection depends on the clinicians having a high index of suspicion and asking appropriate screening questions.

Clinical diagnosis of synucleinopathic subjects that are affected by or at risk of developing Lewy body disease can be supported by neuroimaging investigations. Changes associated with DLBD include preservation of hippocampal, and medialtemporal lobe volume on MRI and occipital hypoperfusion on SPECT. Other features, such as generalized atrophy, white matter changes, and rates of progression of whole brain atrophy are not helpful in differential diagnosis. Dopamine transporter loss in the caudate and putamen, a marker of nigrostriatal degeneration, can be detected by dopamenergic SPECT and can prove helpful in clinical differential diagnosis. A sensitivity of 83% and specificity of 100% has been reported for an abnormal scan with an autopsy diagnosis of DLBD.

Consensus criteria for diagnosing DLBD include ubiquitin immunohistochemistry for Lewy body identification and staging into three categories; brain stem predominant, limbic, or neocortical, depending on the numbers and distribution of Lewy bodies. The recently-developed α-synuclein immunohistochemistry can visualize more Lewy bodies and is also better at indicating previously under recognized neurotic pathology, termed Lewy neurites.

In most patients with DLBD, there are no genetic mutations in the α-synuclein or other Parkinson's disease-associated genes. Pathological up-regulation of normal, wild-type α-synuclein due to increased mRNA expression is a possible mechanism, or Lewy bodies may form because α-synuclein becomes insoluble or more able to aggregate. Another possibility is that α-synuclein is abnormally processed, for example, by a dysfunctional proteasome system and that toxic “proto fibrils” are therefore produced. Sequestering of these toxic fibrils into Lewy bodies could reflect an effort by the neurons to combat biological stress inside the cell, rather than their simply being neurodegenerative debris.

Target symptoms for the accurate diagnosis of DLBD can include extrapyramidal motor features, cognitive impairment, neuropsychiatric features (including hallucinations, depression, sleep disorder, and associated behavioral disturbances), or autonomic dysfunction.

### Multiple System Atrophy

MSA is a neurodegenerative disease marked by a combination of symptoms; affecting movement, cognition, autonomic and other body functions, hence the label “multiple system atrophy”. The cause of MSA is unknown. Symptoms of MSA vary in distribution of onset and severity from person to person. Because of this, the nomenclature initially included three distinct terms: Shy-Drager syndrome, striatonigral degeneration (SD), and olivopontocerebellar atrophy (OPCA). These terms have been replaced by the nomenclature MSA-C (MSA with a cerebellar phenotype) and MSA-P (MSA with a parkinsonian phenotype).

In Shy-Drager syndrome, the most prominent symptoms are those involving the autonomic system, e.g., blood pressure, urinary function, and other functions not involving conscious control. Striatonigral degeneration causes predominately parkinsonism (slowed movements and rigidity), while OPCA principally affects balance, coordination, and speech. The symptoms for MSA typically include orthostatic hypotension, impotence, urinary difficulties, constipation, and speech and swallowing difficulties.

The initial diagnosis of MSA is usually made by carefully interviewing the patient and performing a physical examination. Several types of brain imaging, including computer tomography, scans, magnetic resonance imaging (MRI), and positron emission tomography (PET), can be used as corroborative studies. An incomplete and relatively poor response to dopamine replacement therapy, such as SINEMET (levodopa/carbidopa), may be a clue that a presentation of bradykinesia and rigidity (parkinsonism) is not due to PD. A characteristic involvement of multiple brain systems with prominent autonomic dysfunction is a defining feature of MSA and one that at autopsy confirms the diagnosis. The presence of glial cytoplasmic inclusions containing α-synuclein is pathognomic of MSA. In comparison to Parkinson's disease, in addition to the poor response to Sinemet, there are a few other observations that are strongly suggested for MSA, such as postural instability, low blood pressure on standing (orthostatic hypotension) and high blood pressure when lying down (supine hypertension), urinary difficulties, impotence, constipation, speech and swallowing difficulties out of proportion to slowness and rigidity.

### Indatraline Derivatives as α-Synuclein Aggregation Inhibitors

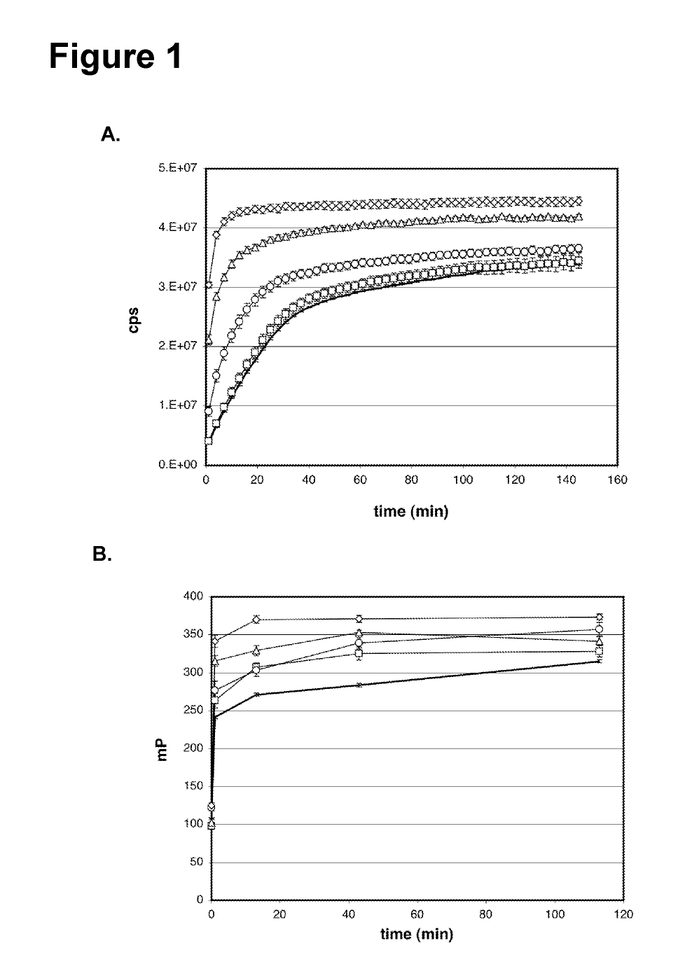

The present invention is at least in part due to the discovery that, in the presence of certain organic solvents, α-synuclein populates conformations that do not lay on the pathway to beta sheet-rich protofibrillar and fibrillar aggregates. Importantly, stabilization of such off-pathway conformers can reduce the rate of α-synuclein fibrillization. For instance, sequence changes that promote formation of helical conformers also reduce the rate of α-synuclein fibrillization. A novel screen was devised to identify small molecules capable of promoting off-pathway conformers of α-synuclein. A screen of 640 central nervous system (CNS-active) compounds, including many approved CNS drugs, identified an active compound, indatraline (INDAT), a monoamine reuptake inhibitor that has been widely used as a research tool. See FIG. 1. Solution NMR studies demonstrate that indatraline binds at a sequence near the C-terminus of alpha-synuclein, a region that has been implicated in the aggregation process (Bertoncini, C. W., Proc Natl Acad Sci, 102, 1430-5, 2005).

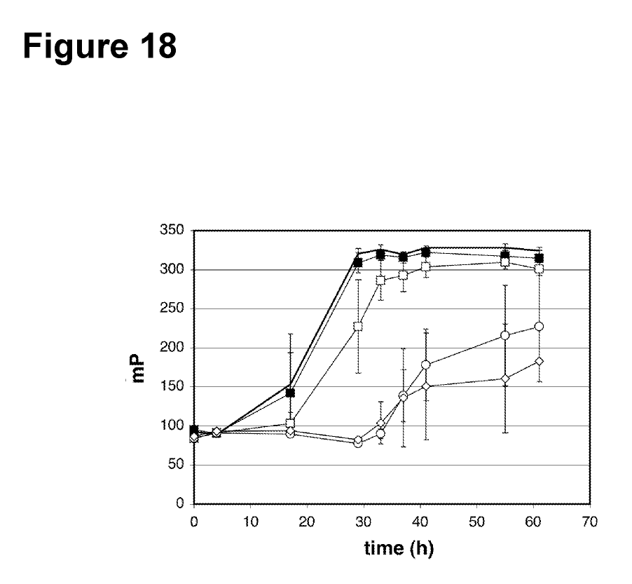

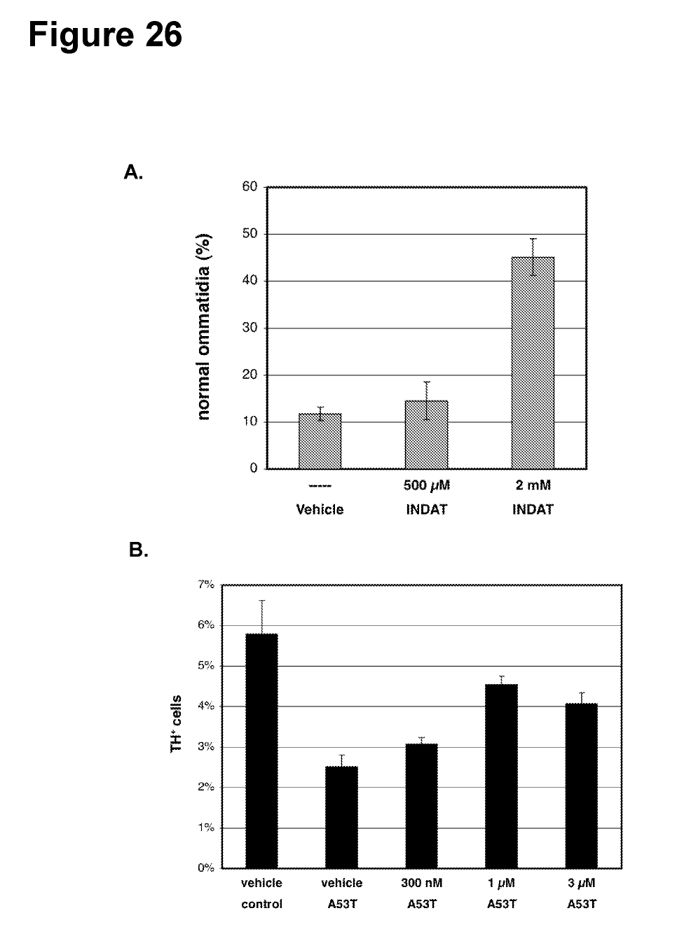

The present invention emanated partly from a series of increasingly complex assays performed with indatraline. First, both reduced the rate of alpha-synuclein fibrillization (and nucleation) in vitro (FIG. 18). Second, indatraline reduced the toxicity of alpha-synuclein in two systems: (1) drosophila retina (FIG. 26A, transgenic drosophila model), and (2) dopaminergic neurons (primary rat embryonic midbrain culture (IC₅₀ ca. 500 nM, FIG. 26B). Viable drug candidates for treating synucleinopathies and other neurodegenerative disorders should preferably be active at brain concentrations at which the disruption of monoamine reuptake is minimal The development of such inventive compounds is described herein.

The present invention provides a novel system for treating synucleinopathic subjects (e.g., Parkinson's disease) or patients with other neurodegenerative diseases. In certain embodiments, the invention includes methods of treating or preventing a synucleinopathy based on the discovery that indatraline inhibits the aggregation of α-synuclein. In certain embodiments, the invention includes methods of treating Parkinson's Disease (PD), diffuse Lewy body disease (DLBD), multiple system atrophy (MSA), and neuronal brain iron accumulation syndrome with α-synuclein deposition, with a compound of formula I. The invention provides methods for treating a subject with a synucleinopathy or other neurodegenerative disease, comprising the step of administering to the subject a therapeutically effective amount of a compound of formula I to inhibit the aggregation of α-synuclein.

In certain embodiments, the invention includes methods of treating a subject with a neurodegenerative disease such as amyotrophic lateral sclerosis (ALS), Huntington's Disease (HD), or Alzheimer's Disease (AD), with a compound of formula I. Without wishing to be bound by any particular theory or mechanism of action, the methods of the invention are useful in preventing or decreasing the accumulation, aggregation, and/or toxicity of α-synuclein. In certain embodiments, the treatment methods decrease the aggregation of α-synuclein. In certain er embodiments, the treatment methods inhibit the aggregation of α-synuclein. In certain embodiments, the methods are useful in reducing the toxicity of aggregations of α-synuclein. In still other embodiments, the methods are useful in decreasing levels of insoluble α-synuclein and/or increasing clearance of α-synuclein. In certain embodiments, the subject is a vertebrate. In certain embodiments, the subject is a mammal. In preferred embodiments, the subject is a human. The human may be male or female, and the human may be at any stage of development. The subject may be a test animal such as a mouse, rat, or dog.

Based on these discoveries, the invention also provides methods of using indatraline analogs, both in vitro and in vivo to prevent or reduce the aggregation of α-synuclein. The invention also provides pharmaceutical compositions and preparations comprising a compound of formula I. Kits containing the inventive indatraline analogs or pharmaceutical compositions thereof are also provided. The present invention also provides methods for preparing indatraline derivatives having activity as inhibitors of α-synuclein aggregation. Combination therapies with other agents such as farnesyl transferase inhibitors are also provided.

### Compounds

The present invention provides indatraline derivatives of formula I:

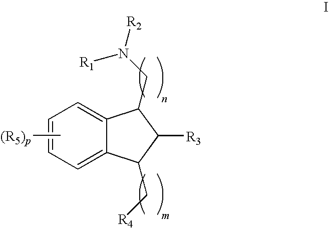

wherein

n is an integer between 0 and 6, inclusive;

m is an integer between 0 and 6, inclusive;

p is an integer between 0 and 4, inclusive;

R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{A}; —CO₂R_{A}; —C(═O)N(R_{A})₂; or —C(R_{A})₃; wherein each occurrence of R_{A} is independently a hydrogen, a protecting group as described above, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; —C(═O)N(R_{B})₂; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is substituted or unsubstituted, branched or unbranched aryl; or substituted or unsubstituted, branched or unbranched heteroaryl;

R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; or a derivative, analog, isomer, solvate, salt or other pharmaceutically acceptable forms thereof.

For certain compounds of formula I, the stereochemistry is defined as follows:

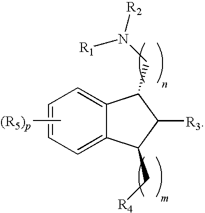

The configuration may be referred to as trans because the substituents are opposite each other with respect to the plane of the bicyclic core.

For certain compounds of formula I, the stereochemistry is defined as follows:

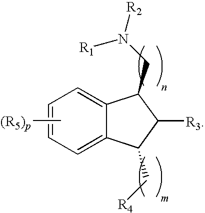

The configuration may be referred to as trans because the substituents are opposite each other with respect to the plane of the bicyclic core.

For other compounds of formula I, the stereochemistry is defined as follows:

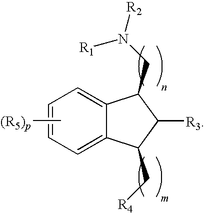

The configuration may be referred to as cis because the substituents are together on one side of the plane of the bicyclic core.

For other compounds of formula I, the stereochemistry is defined as follows:

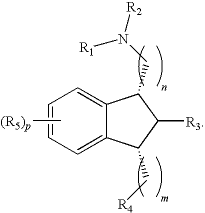

The configuration may be referred to as cis because the substituents are together on one side of the plane of the bicyclic core.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain particular compounds, R₁ and R₂ are both methyl. In certain embodiments, R₁ is hydrogen, and R₂ is methyl. In certain embodiments, R₁ is hydrogen, and

R₂ is benzyl. In certain embodiments, R₁ and R₂ are both benzyl. In certain embodiment, R₁ and R₂ are both hydrogen.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, R₄ is aryl or heteroaryl. In certain embodiments, R₄ is phenyl, naphthyl, indolyl, or pyridinyl. In certain embodiments, R₄ is substituted phenyl. In certain embodiments, R₄ is monosubstituted phenyl. In certain embodiments, R₄ is disubstituted phenyl. In certain embodiments, R₄ is trisubstituted phenyl. In certain embodiments, R₄ is unsubstituted phenyl. In certain embodiments, R₄ is 3,4-dichlorophenyl. In certain embodiments, R₄ is 4-chlorophenyl. In certain embodiments, R₄ is 3-chlorophenyl. In certain embodiments, R₄ is 3,4-difluorophenyl. In certain embodiments, R₄ is 4-fluorophenyl. In certain embodiments, R₄ is 3-fluorophenyl. In certain embodiments, R₄ is substituted naphthyl. In certain embodiments, R₄ is unsubstituted naphthyl. In certain embodiments, R₄ is substituted indolyl. In certain embodiments, R₄ is unsubstituted indolyl. In certain embodiments, R₄ is unsubstituted pyridinyl. In certain embodiments, R₄ is substituted pyridinyl.

Exemplary compounds of the invention include:

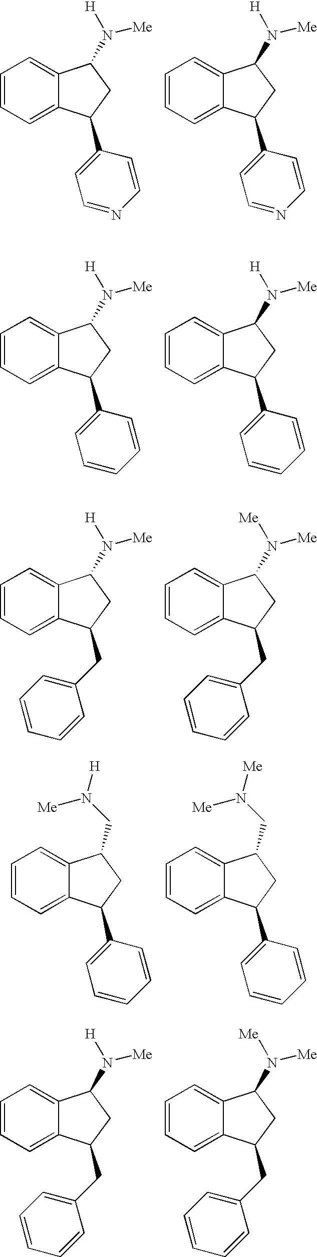

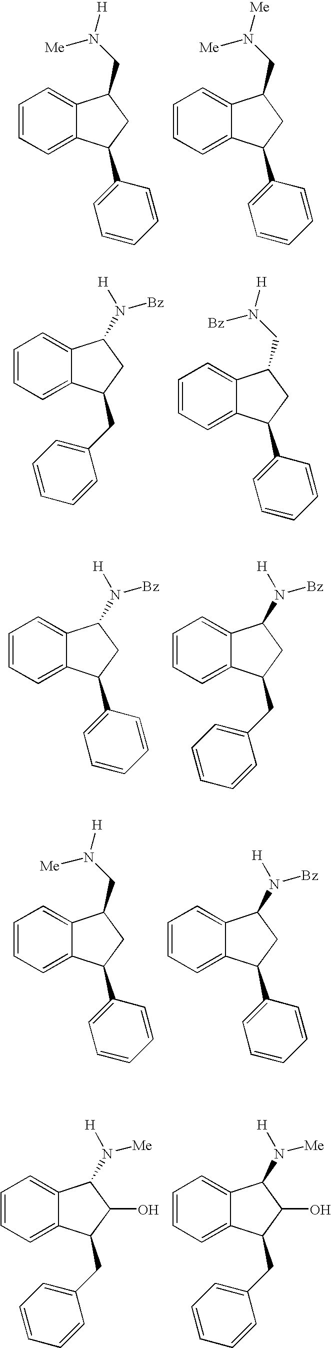

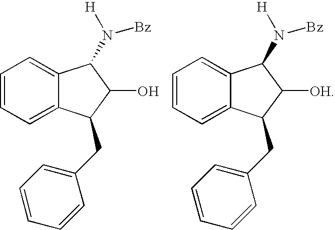

Compounds useful in the present invention include compounds of the formula II:

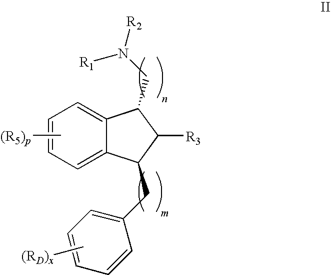

wherein

n is an integer between 0 and 6, inclusive;

m is an integer between 0 and 6, inclusive;

x is an integer between 0 and 5, inclusive;

each of R₁, R₂, R₃, and R₅ is defined above;

R_{D} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable forms thereof.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain particular compounds, R₁ and R₂ are both methyl. In certain embodiments, R₁ is hydrogen, and R₂ is methyl. In certain embodiments, R₁ is hydrogen, and R₂ is benzyl. In certain embodiments, R₁ and R₂ are both benzyl. In certain embodiment, R₁ and R₂ are both hydrogen.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain classes of compounds of formula II, x is an integer between 0 and 5, and R_{D} is halogen. For certain compounds, x is 2; and R_{D} is chloro. For certain compounds, x is 1; and R_{D} is chloro. For certain compounds, x is 2; and R_{D} is fluoro. For certain compounds, x is 1; and R_{D} is chloro.

Compounds useful in the present invention include compounds of the formula:

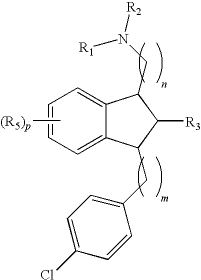

wherein

n is an integer between 0 and 6, inclusive;

m is an integer between 0 and 6, inclusive;

p is an integer between 0 and 4, inclusive; and

each of R₁, R₂, R₃, and R₅ is defined above.

In certain embodiments, the stereochemistry is defined as follows:

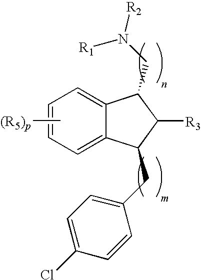

and in certain embodiments the corresponding enantiomer.

In certain embodiments, the stereochemistry is defined as follows:

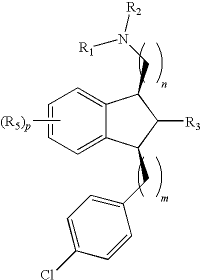

and in certain embodiments the corresponding enantiomer.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, n and m are as described above, R₁ and R₂ are each independently methyl, ethyl, propyl, or butyl. In certain particular compounds, R₁ and R₂ are both methyl. For other compounds, R₁ is H; and R₂ is methyl. In certain embodiments, R₁ and R₂ are both hydrogen. In certain embodiments, R₁ is hydrogen, and R₂ is benzyl. In certain embodiments, R₁ and R₂ are both benzyl.

Exemplary compounds of the invention include:

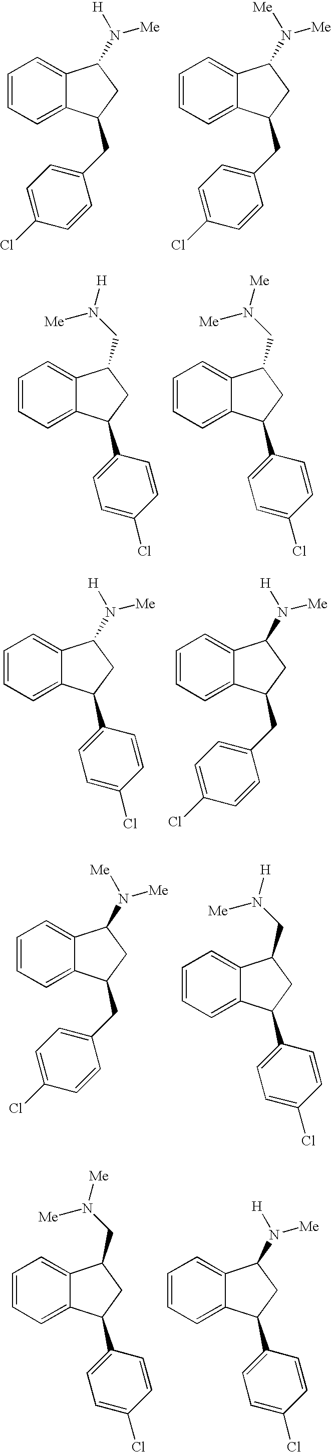

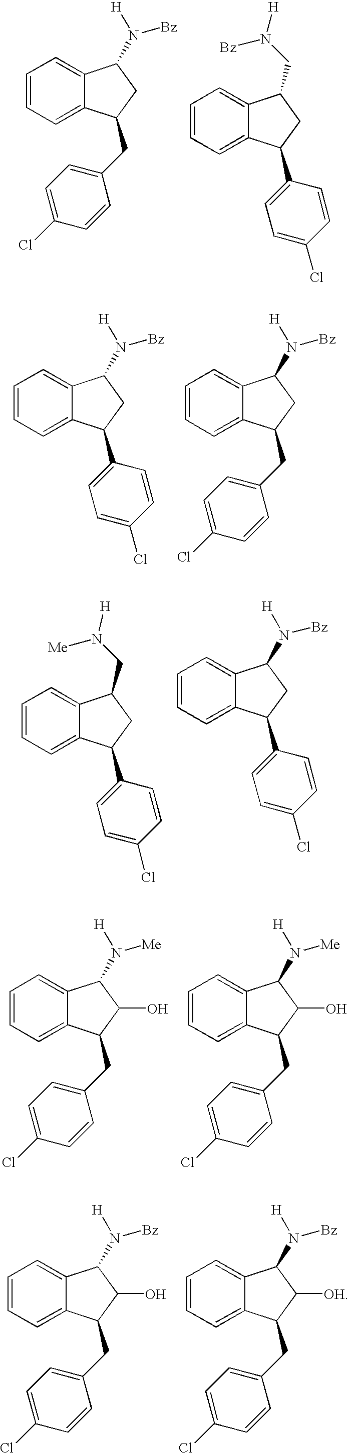

Compounds useful in the present invention include compounds of the formula:

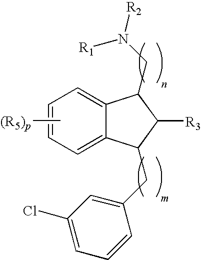

wherein

n is an integer between 0 and 6, inclusive;

m is an integer between 0 and 6, inclusive;

p is an integer between 0 and 4, inclusive; and

each of R₁, R₂, R₃, and R₅ is defined above.

In certain embodiments, the stereochemistry is defined as follows:

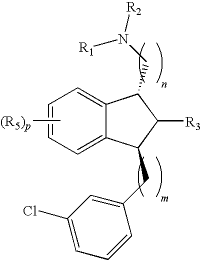

and in certain embodiments the corresponding enantiomer.

In certain embodiments, the stereochemistry is defined as follows:

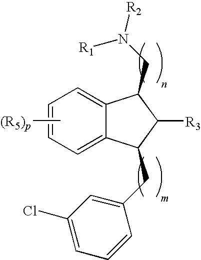

and in certain embodiments the corresponding enantiomer.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, n and m are as described above, R₁ and R₂ are each independently methyl, ethyl, propyl, or butyl. In certain particular compounds, R₁ and R₂ are both methyl. For other compounds, R₁ is H; and R₂ is methyl. In certain embodiments, R₁ and R₂ are both hydrogen. In certain embodiments, R₁ is hydrogen, and R₂ is benzyl. In certain embodiments, R₁ and R₂ are both benzyl.

Exemplary compounds of the invention include:

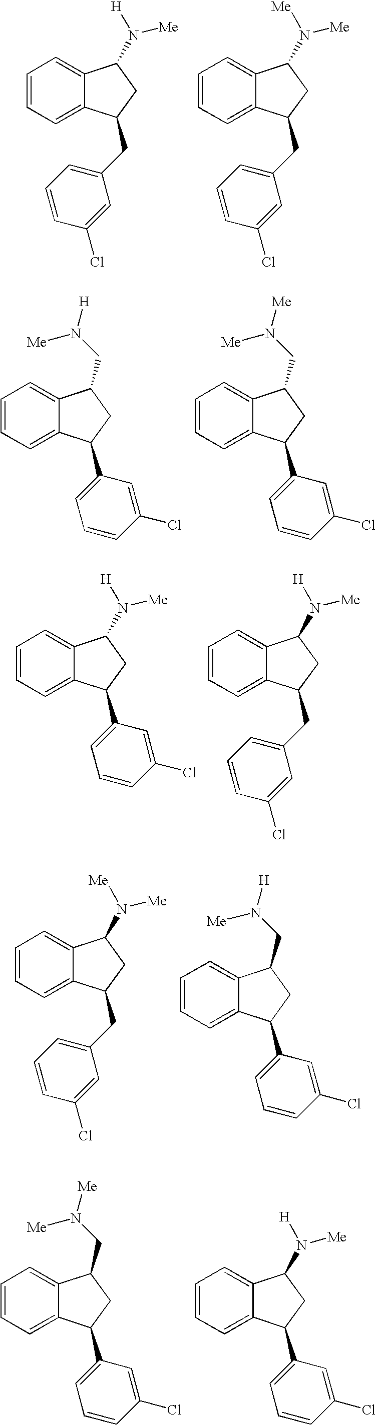

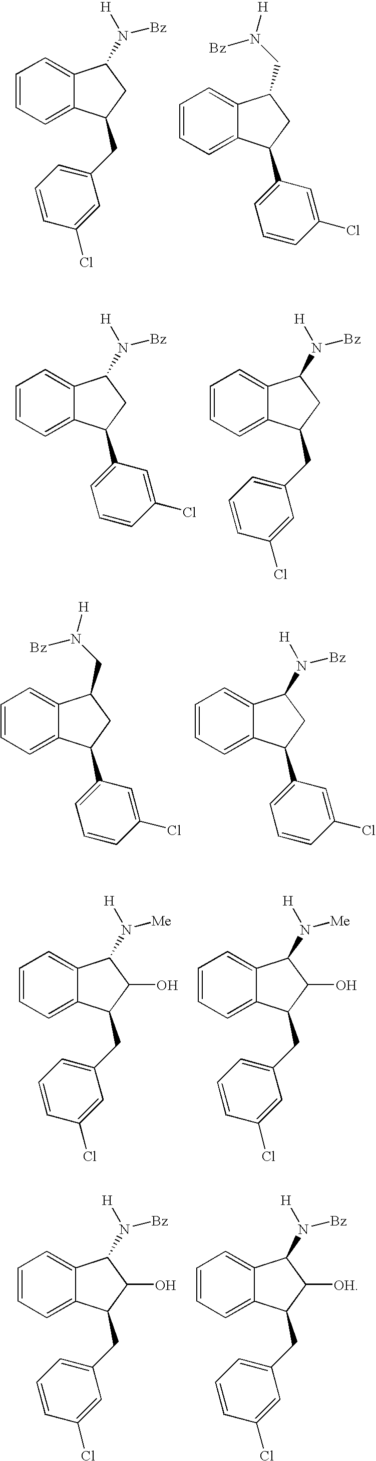

Compounds useful in the present invention include compounds of the formula III:

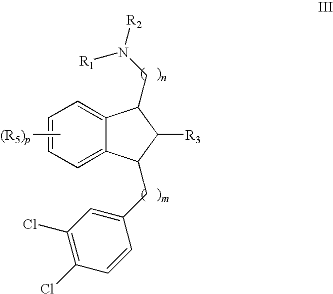

wherein

n is an integer between 0 and 6, inclusive;

m is an integer between 0 and 6, inclusive;

p is an integer between 0 and 4, inclusive

each of R₁, R₂, R₃, and R₅ is defined above.

For certain compounds of formula III, the stereochemistry is defined as follows:

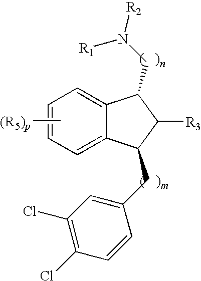

and in certain embodiments the corresponding enantiomer.

For other compounds of formula III, the stereochemistry is defined as follows:

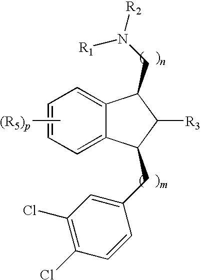

and in certain embodiments the corresponding enantiomer.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, n and m are as described above, R₁ and R₂ are each independently methyl, ethyl, propyl, or butyl. In certain particular compounds, R₁ and R₂ are both methyl. For other compounds, R₁ is H; and R₂ is methyl. In certain embodiments, R₁ and R₂ are both hydrogen. In certain embodiments, R₁ is hydrogen, and R₂ is benzyl. In certain embodiments, R₁ and R₂ are both benzyl.

Exemplary compounds of the invention include:

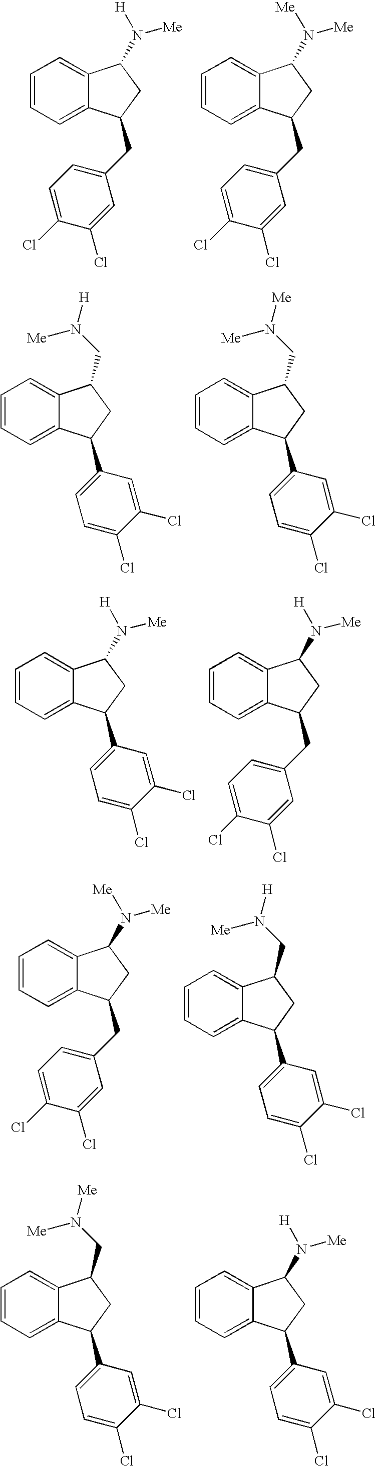

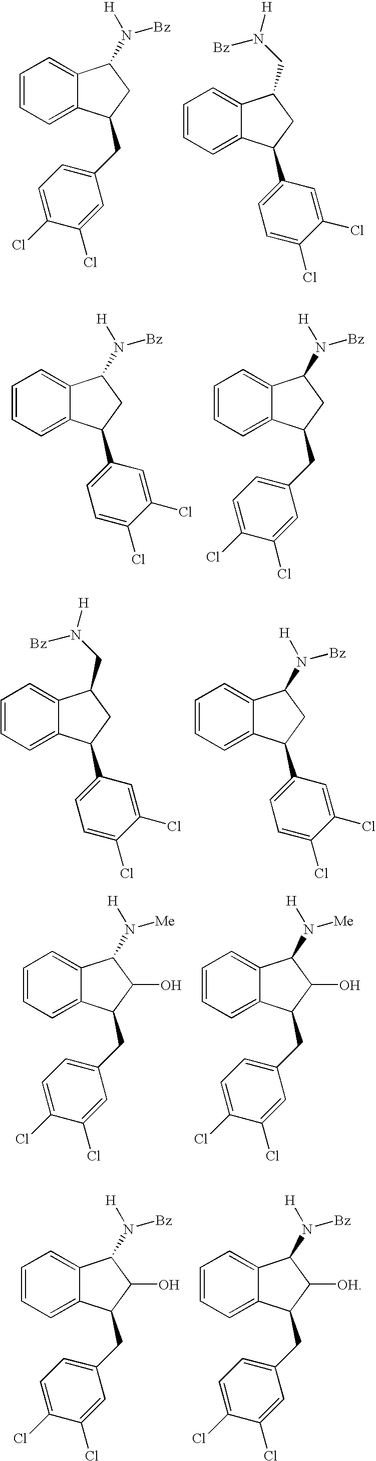

Compounds useful in the present invention include compounds of the formula:

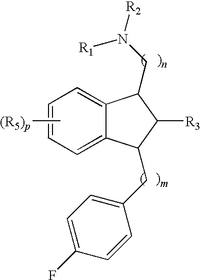

wherein

n is an integer between 0 and 6, inclusive;

m is an integer between 0 and 6, inclusive;

p is an integer between 0 and 4, inclusive

each of R₁, R₂, R₃, and R₅ is defined above.

In certain embodiments, the stereochemistry is defined as follows:

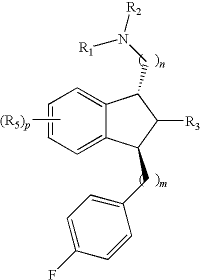

and in certain embodiments the corresponding enantiomer.

In certain embodiments, the stereochemistry is defined as follows:

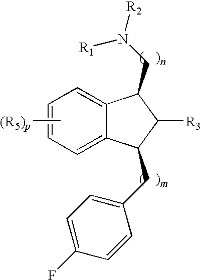

and in certain embodiments the corresponding enantiomer.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, n and m are as described above, R₁ and R₂ are each independently methyl, ethyl, propyl, or butyl. In certain particular compounds, R₁ and R₂ are both methyl. For other compounds, R₁ is H; and R₂ is methyl. In certain embodiments, R₁ and R₂ are both hydrogen. In certain embodiments, R₁ is hydrogen, and R₂ is benzyl. In certain embodiments, R₁ and R₂ are both benzyl.

Exemplary compounds of the invention include:

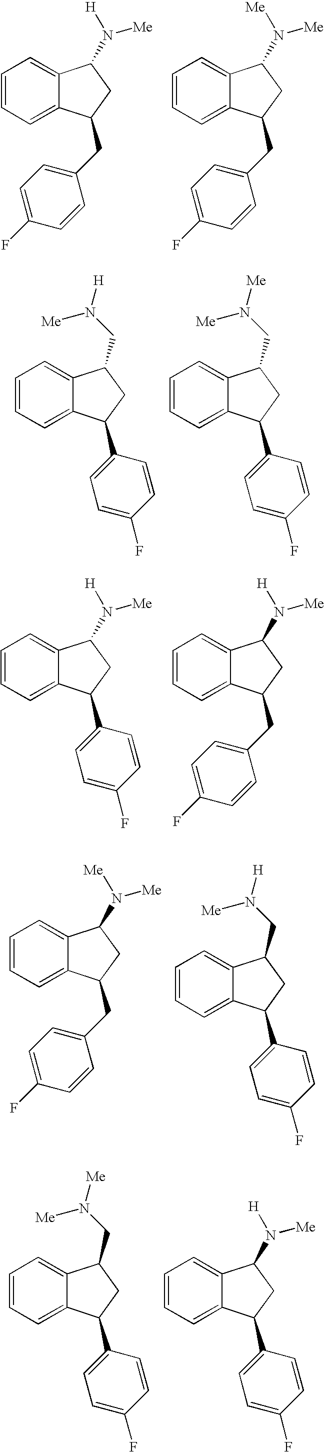

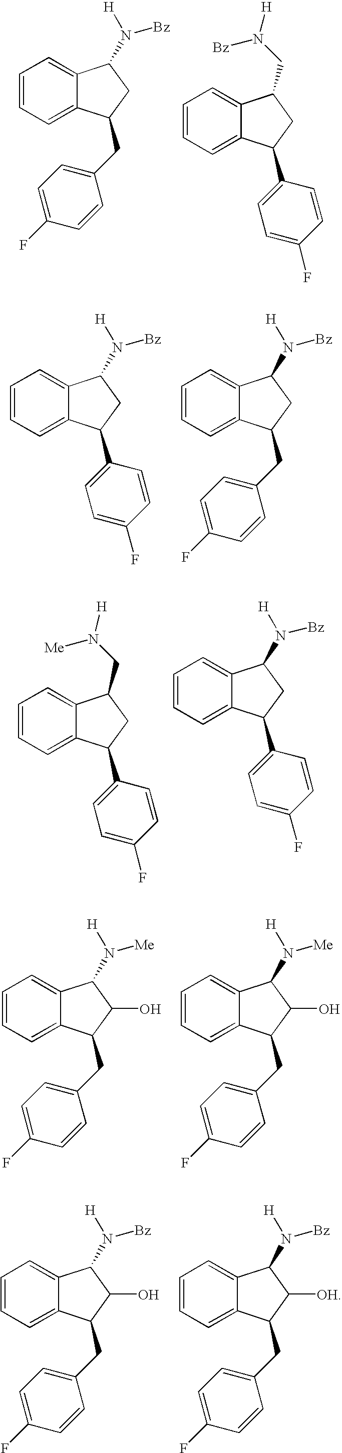

Compounds useful in the present invention include compounds of the formula:

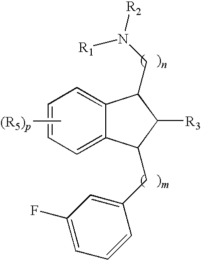

wherein

n is an integer between 0 and 6, inclusive;

m is an integer between 0 and 6, inclusive;

p is an integer between 0 and 4, inclusive

each of R₁, R₂, R₃, and R₅ is defined above.

In certain embodiments, the stereochemistry is defined as follows:

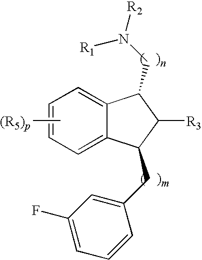

and in certain embodiments the corresponding enantiomer.

In certain embodiments, the stereochemistry is defined as follows:

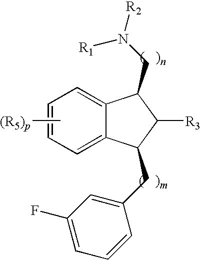

and in certain embodiments the corresponding enantiomer.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, n and m are as described above, R₁ and R₂ are each independently methyl, ethyl, propyl, or butyl. In certain particular compounds, R₁ and R₂ are both methyl. For other compounds, R₁ is H; and R₂ is methyl. In certain embodiments, R₁ and R₂ are both hydrogen. In certain embodiments, R₁ is hydrogen, and R₂ is benzyl. In certain embodiments, R₁ and R₂ are both benzyl.

Exemplary compounds of the invention include:

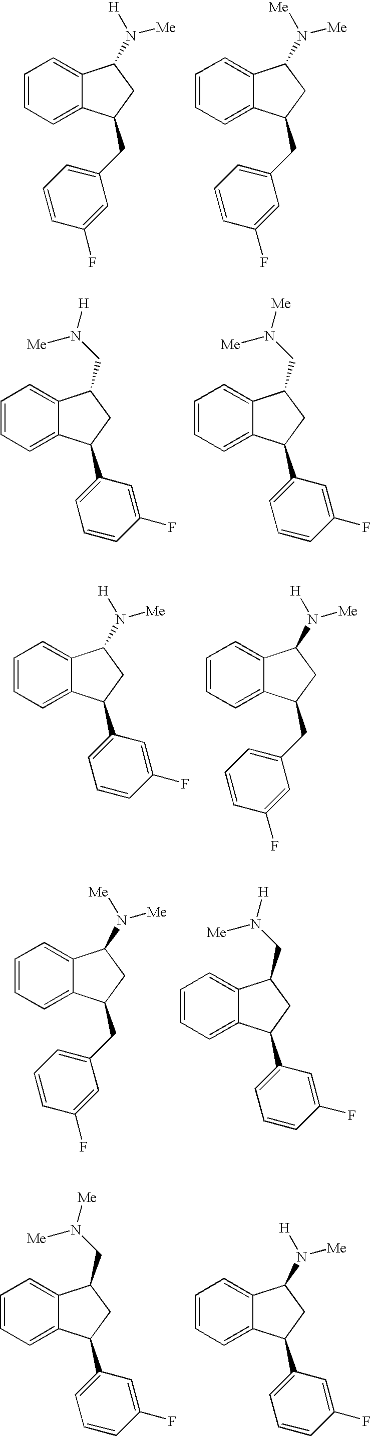

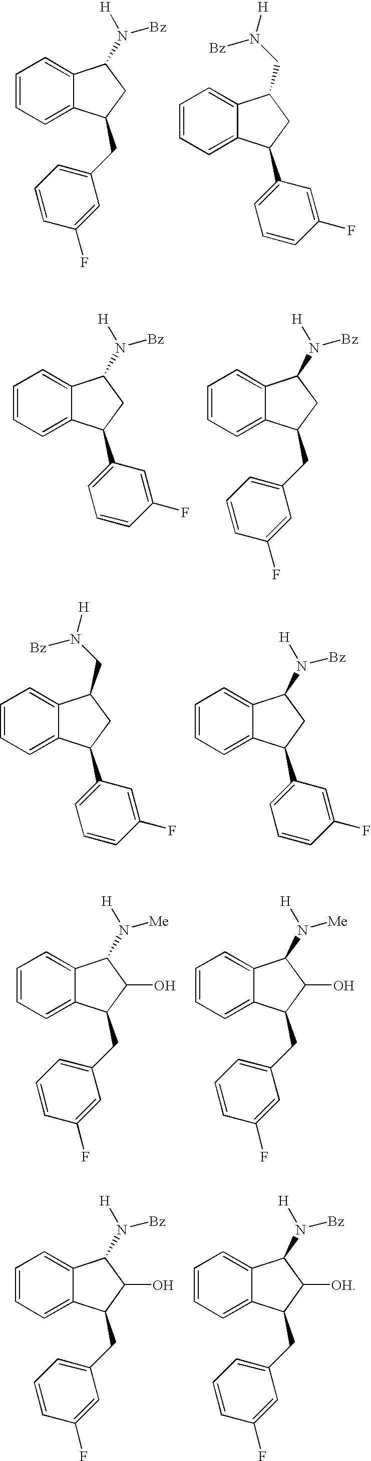

Compounds useful in the present invention include compounds of the formula:

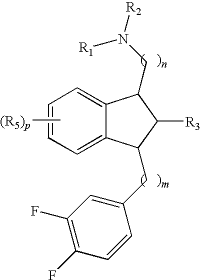

wherein

n is an integer between 0 and 6, inclusive;

m is an integer between 0 and 6, inclusive;

p is an integer between 0 and 4, inclusive; and

each of R₁, R₂, R₃, and R₅ is defined above.

In certain embodiments, the stereochemistry of the compound is defined as follows:

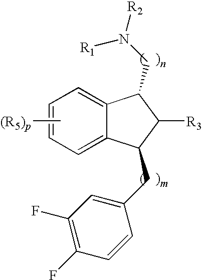

and in certain embodiments the corresponding enantiomer.

In certain embodiments, the stereochemistry is defined as follows:

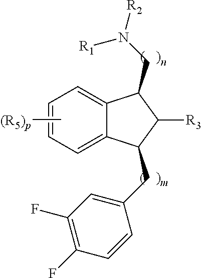

and in certain embodiments the corresponding enantiomer.

In certain embodiments, n is 0. In certain embodiments, n is 1. In certain embodiments, n is 2. In certain embodiments, n is 3.

In certain embodiments, m is 0. In certain embodiments, m is 1. In certain embodiments, m is 2. In certain embodiments, m is 3.

In certain embodiments, at least one of n and m is 1. In certain embodiments, at least one of n and m is 2. In certain embodiments, at least one of n and m is 3. In certain embodiments, at least one of n and m is 0. In certain embodiments, both n and m are not 0.

In certain embodiments, both n and m are greater than or equal to 1. In certain embodiments, both n and m are greater than or equal to 2. In certain embodiments, both n and m are greater than or equal to 3.

In certain embodiments, R₁ is H, C₁-C₆ alkyl, acyl, or a protecting group as defined above. In certain embodiments, R₁ is methyl, ethyl, propyl, or butyl. In certain embodiments, R₁ is methyl. In certain embodiments, R₁ is hydrogen. In certain embodiments, R₁ is hydroxyethyl. In certain embodiments, R₁ is benzyl.

In certain embodiments, R₂ is H, C₁-C₆ alkyl, acyl, or a protecting group as defined above. In certain embodiments, R₂ is methyl, ethyl, propyl, or butyl. In certain embodiments, R₂ is methyl. In certain embodiments, R₂ is hydroxyethyl. In certain embodiments, R₂ is hydrogen. In certain embodiments, R₂ is benzyl.

In certain embodiments, R₃ is hydrogen. In certain embodiments, R₃ is hydroxyl. In certain embodiments, R₃ is —N(R_{C})₂. In certain embodiments, R₃ is —NH₂. In certain embodiments R₃ is C₁-C₆ alkyl.

In certain embodiments, n and m are as described above, R₁ and R₂ are each independently methyl, ethyl, propyl, or butyl. In certain particular compounds, R₁ and R₂ are both methyl. For other compounds, R₁ is H; and R₂ is methyl. In certain embodiments, R₁ and R₂ are both hydrogen. In certain embodiments, R₁ is hydrogen, and R₂ is benzyl. In certain embodiments, R₁ and R₂ are both benzyl.

Exemplary compounds of the invention include:

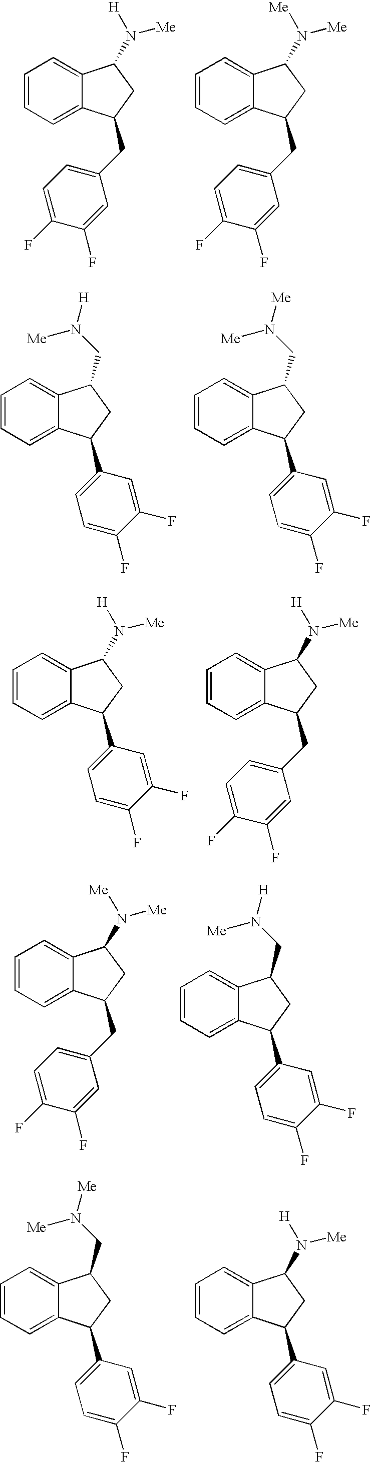

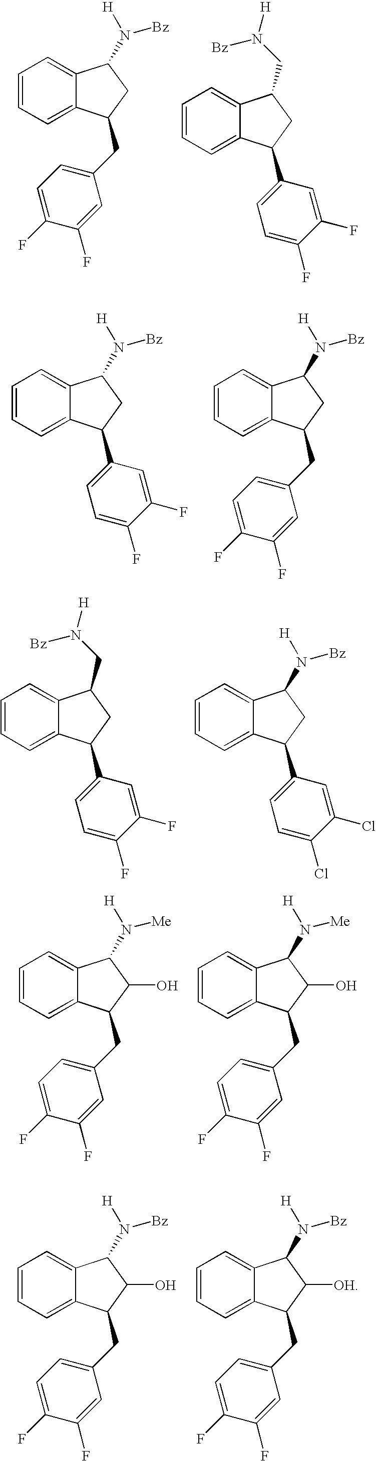

Compounds useful in the present invention include compounds of the formula IV:

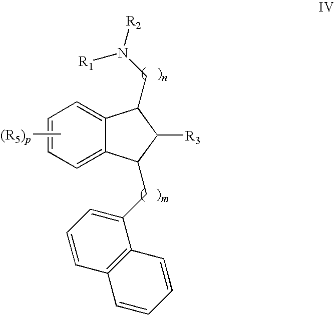

wherein

n is an integer between 0 and 6, inclusive;

m is an integer between 0 and 6, inclusive;

p is an integer between 0 and 4, inclusive; and each of R₁, R₂, R₃, and R₅ is defined above.

For certain compounds of formula IV, the stereochemistry is defined as follows:

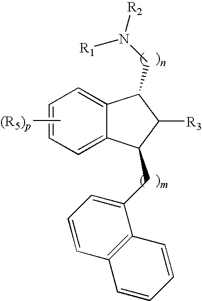

and in certain embodiments the corresponding enantiomer.

For other compounds of formula IV, the stereochemistry is defined as follows:

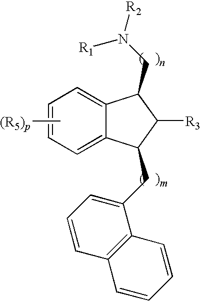

and in certain embodiments the corresponding enantiomer.

In certain genera of compounds of formula IV, n and m are as described above, R₁ and R₂ are each independently methyl, ethyl, propyl, or butyl. In certain particular compounds, R₁ and R₂ are methyl. For other compounds, R₁ is H and R₂ is methyl. In certain embodiments, both R₁ and R₂ are hydrogen.

Exemplary compounds of the invention include:

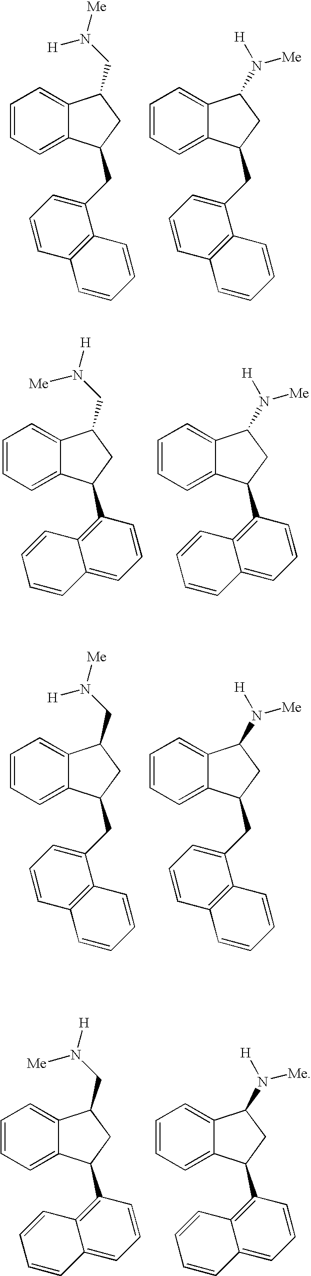

Compounds useful in the present invention include compounds of the formula V:

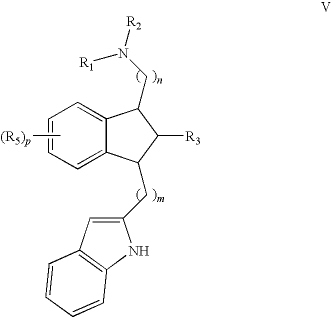

wherein

n is an integer between 0 and 6, inclusive;

m is an integer between 0 and 6, inclusive;

p is an integer between 0 and 4, inclusive; and

each of R₁, R₂, R₃, and R₅ is defined above.

For certain compounds of formula V, the stereochemistry is defined as follows:

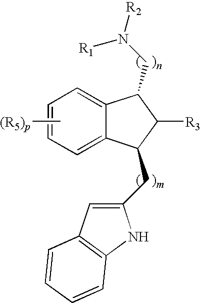

and in certain embodiments the corresponding enantiomer.

For other compounds of formula V, the stereochemistry is defined as follows:

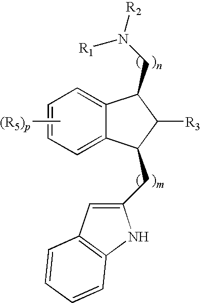

and in certain embodiments the corresponding enantiomer.

In certain classes of compounds of formula V, n and m are as described above, R₁ and R₂ are each independently methyl, ethyl, propyl, or butyl. In certain particular compounds, R₁ and R₂ are methyl. For other compounds, R₁ is H and R₂ is methyl.

Exemplary compounds of the invention include:

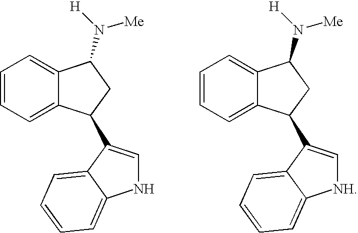

Compounds useful in the present invention include compounds of the formula VI:

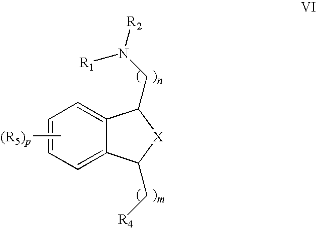

wherein

n is an integer between 0 and 6, inclusive;

m is an integer between 0 and 6, inclusive;

p is an integer between 0 and 4, inclusive;

each of R₁, R₂, R₃, and R₅ is defined above;

X is O, S, NH, or NR₆; wherein R₆ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{A}; —CO₂R_{A}; —C(═O)N(R_{A})₂; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

For certain compounds of formula VI, the stereochemistry is defined as follows:

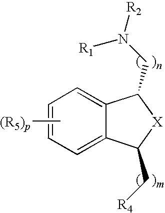

and in certain embodiments the corresponding enantiomer.

For other compounds of formula VI, the stereochemistry is defined as follows:

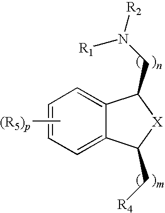

and in certain embodiments the corresponding enantiomer.

In certain classes of compounds of formula VI, n and m are as described above, and X is NH or NR₆; wherein R₆ is acyl or C₁-C₆ alkyl. In certain compounds useful in this invention, X is NH, and R₁ and R₂ are each independently methyl, ethyl, propyl, or butyl. In certain particular compounds, R₁ and R₂ are methyl. For other compounds, R₁ is H and R₂ is methyl.

Exemplary compounds of the invention include:

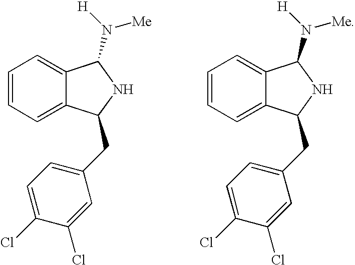

### Uses

The inventive indatraline derivatives have been discovered to prevent the aggregation of α-synuclein. The inventive compounds may be screened for their ability to prevent the aggregation of α-synuclein using techniques known in the art. Particular assays for measuring a compound's ability to prevent the aggregation of α-synuclein are described in the Examples section below. In certain embodiments, the assays involves testing for the aggregation of α-synuclein in hexafluoroisopropanol. In other embodiments, the assay involves testing for the aggregation of α-synuclein in an aqueous solution.

In certain embodiments, the inventive compounds of formula I are not inhibitors of monoamine transporters, or they do not substantially inhibit monoamine transport (e.g. less than 10%, 20%, 30%, 40%, 50%, 60%, 70%, 80%, or 90% inhibition). In certain embodiments, the inventive compounds of formula I inhibit monoamine transporters to some degree, but the ratio of relative α-synuclein activity to relative monamine transporter activity is a number greater than one. In certain embodiments the number is greater than ten. In certain embodiments the number is greater than 100.

In certain embodiments, the inventive compounds of formula I have minimal affinity for the P-glycoprotein. In certain embodiments, said protein is human P-glycoprotein.

In certain embodiments, the inventive compounds of formula I minimally inhibit cytochrome P450 enzymes (CYP). In certain embodiments, the inhibition is less than 50%. In certain embodiments, the inhibition is less than 25%. In certain embodiments, the inhibition is less than 10%. In certain embodiments, the inhibition is less than 5%. In certain embodiments, the enzyme is CYP1A2. In certain embodiments, the enzyme is CYP3A4.

In certain embodiments, the inventive compounds of formula I have minimal in vivo toxicity. In certain embodiments, toxicity is measured as whole-cell hepatotoxicity. In certain embodiments, lactate dehydrogenase (LDH) release is used as a surrogate measure. In certain embodiments, reduction in cellular proliferation is used as a surrogate measure.

In certain embodiments, the inventive compounds of formula I will have optimal metabolic stability. In certain embodiments, the ACTIVTox™ is used to measure metabolic stability.

In certain embodiments, the inventive compounds of formula I may be used in the treatment of Parkinson's Disease. In certain embodiments, a compound of formula I is used in the treatment or prevention of Parkinson's Disease with or without other therapy.

In certain embodiments, the inventive compounds of formula I may be used in the treatment of diffuse Lewy Body disease. In certain embodiments, a compound of formula I is used in the treatment or prevention of diffuse Lewy Body disease with or without other therapy.

In certain embodiments, the inventive compounds of formula I may be used in the treatment of multiple system atrophy. In certain embodiments, a compound of formula I is used in the treatment or prevention of multiple system atrophy with or without other therapy.

In certain embodiments, the inventive compounds of formula I may be used in the treatment of a neuronal brain iron accumulation disorder. In certain embodiments, a compound of formula I is used in the treatment or prevention of a neuronal brain iron accumulation disorder with or without other therapy.

In certain embodiments, the inventive compounds of formula I may be used in the treatment of amyotrophic lateral sclerosis. In certain embodiments, a compound of formula I is used in the treatment or prevention of amyotrophic lateral sclerosis with or without other therapy.

In certain embodiments, the inventive compounds of formula I may be used in the treatment of Huntington's disease. In certain embodiments, a compound of formula I is used in the treatment or prevention of Huntington's disease with or without other therapy.

In certain embodiments, the inventive compounds of formula I may be used in the treatment of Alzheimer's disease. In certain embodiments, a compound of formula I is used in the treatment or prevention of Alzheimer's disease with or without other therapy. In certain embodiments, the subject has Alzheimer's disease with Lewy bodies.

The therapeutically effective amount of the inventive compound included in the therapy will vary depending on the patient, the disease being treated, extent of disease, the route of administration, other medications being administered to the patient, desired outcome, etc. In certain embodiments, the inventive compound is administered in the range of approximately 0.0001 mg/kg body weight to approximately 25 mg/kg body weight. In certain embodiments, the inventive compound is administered in the range of approximately 1 mg/kg body weight to approximately 25 mg/kg body weight. In certain embodiments, the inventive compound is administered in the range of approximately 10 mg/kg body weight to approximately 20 mg/kg body weight. In certain embodiments, the inventive compound is administered in the range of approximately 1 mg/kg body weight to approximately 10 mg/kg body weight. In certain embodiments, the inventive compound is administered in the range of approximately 1 mg/kg body weight to approximately 5 mg/kg body weight. In certain embodiments, the inventive compound is administered in the range of approximately 0.001 mg/kg body weight to approximately 1 mg/kg body weight. In certain embodiments, the inventive compound is administered in the range of approximately 0.001 mg/kg body weight to approximately 0.1 mg/kg body weight. In certain embodiments, the inventive compound is administered in the range of approximately 0.01 mg/kg body weight to approximately 0.1 mg/kg body weight. In certain embodiments, approximately 1 mg to approximately 2000 mg of the inventive compound is administered each day. In certain embodiments, approximately 1000 mg to approximately 2000 mg of the inventive compound is administered each day. In certain embodiments, approximately 1 mg to approximately 1000 mg of the inventive compound is administered each day. In certain embodiments, approximately 1 mg to approximately 500 mg of the inventive compound is administered each day. In certain embodiments, approximately 1 mg to approximately 100 mg of the inventive compound is administered each day. In certain embodiments, approximately 1 mg to approximately 50 mg of the inventive compound is administered each day. In certain embodiments, approximately 1 mg to approximately 10 mg of the inventive compound is administered each day. In certain embodiments, approximately 10 mg to approximately 100 mg of the inventive compound is administered each day. In certain embodiments, approximately 25 mg to approximately 100 mg of the inventive compound is administered each day. In certain embodiments, approximately 10 mg to approximately 50 mg of the inventive compound is administered each day. In certain embodiments, approximately 25 mg to approximately 75 mg of the inventive compound is administered each day. As will be appreciated by one of skill in the art, depending on the form of the inventive compound being administered the dosing may vary. The dosages given herein are dose equivalents with respect to the active ingredient. In certain embodiments, the inventive compound is administered parenterally. In certain embodiments, the inventive compound is administered intravenously. In certain embodiments, the inventive compound is administered orally. In certain embodiments, the inventive compound is administered from once a week to four times per day. In certain particular embodiments, the inventive compound is administered once per day. In certain embodiments, the inventive compound is administered twice per day. In certain embodiments, the inventive compound is administered 3-4 times per day.

In certain embodiments, the disease being treated using the inventive compound is Parkinson's disease. In certain embodiments, the disease being treated using the inventive compound is diffuse Lewy body disease. In certain embodiments, the disease being treated using the inventive compound is multiple system atrophy disorder. In certain embodiments, the disease being treated using the inventive compound is pantothenate kinase-associated neurodegeneration. In certain embodiments, the disease being treated using the inventive compound is amyotrophic lateral sclerosis (ALS). In certain embodiments, the disease being treated using the inventive compound is Huntington's disease. In certain embodiments, the disease being treated using the inventive compound is Alzheimer's disease. In certain embodiments, the disease being treated using the inventive compound is Alzheimer's disease with Lewy bodies. In certain embodiments, the disease being treated using the inventive compound is Parkinson's disease. In certain embodiments, the disease being treated using the inventive compound is frontotemporal dementia. In certain embodiments, the disease being treated using the inventive combination is prion disease (e.g., Creutzfeldt Jakob Disease). In certain embodiments, the disease being treated using the inventive combination is Niemann-Pick Type Cl disease. In certain embodiments, the disease being treated using the inventive combination is Gaucher's disease. In certain embodiments, the disease being treated using the inventive combination is progressive supranuclear palsy.

The inventive indatraline derivative may also be used with one or more other pharmaceutical agents. For example, the combination may be used with pharmaceutical agents currently used to treat synucleinopathies or other neurodegenerative diseases, or symptoms arising as side-effects of the disease or of the aforementioned medications.

For example, methods of the invention can be used in combination with medications for treating PD. Levodopa mainly in the form of combination products containing carbodopa and levodopa (Sinemet and Sinemet CR) is the mainstay of treatment and is the most effective agent for the treatment of PD. Levodopa is a dopamine precursor, a substance that is converted into dopamine by an enzyme in the brain. Carbodopa is a peripheral decarboxylase inhibitor that prevents side effects and lower the overall dosage requirement. The starting dose of Sinemet is a 25/100 or 50/200 tablet prior to each meal. Dyskinesias may result from overdose and also are commonly seen after prolonged (e.g., years) use. Direct acting dopamine agonists may have less of this side effect. About 15% of patients do not respond to levodopa. Stalevo (carbodopa, levodopa, and entacapone) is a new combination formulation for patients who experience signs and symptoms of “wearing-off” The formulation combines carbodopa and levodopa (the most widely used agents to treat PD) with entacapone, a catechol-O-methyltransferase inhibitor. While carbodopa reduces the side effects of levodopa, entacapone extends the time levodopa is active in the brain, up to about 10% longer.

Amantadine (SYMMETREL®) is a mild agent thought to work by multiple mechanisms including blocking the re-uptake of dopamine into presynaptic neurons. It also activates the release of dopamine from storage sites and has a glutamate receptor blocking activity. It is used as early monotherapy, and the dosing is typically 200 to 300 mg daily. Amantadine may be particularly helpful in patients with predominant tremor. Side effects may occasionally include ankle swelling and red blotches. It may also be useful in later stage disease to decrease the intensity of drug-induced dyskinesia.

Anticholinergics (trihexyphenidyl, benztropine mesylate, procyclidine, artane, cogentin) do not act directly on the dopaminergic system. Direct-acting dopamine agonists include bromocriptidine (Parlodel), pergolide (Permax), ropinirol (Requip), and pramipexole (Mirapex). These agents cost substantially more than levodopa (Sinemet), and additional benefits are controversial. Depending on which dopamine receptor is being stimulated, D1 and D2 agonist can exert anti-Parkinson effects by stimulating the D1 and D2 receptors, such as Ergolide. Mirapex and Requip are the newer agents. Both are somewhat selective for the dopamine D2 receptor. Direct dopamine agonists, in general, are slightly more likely to produce adverse neuropsychiatric side effects such as confusion than levodopa. Unlike levodopa, direct dopamine agonists do not undergo conversion to dopamine and thus do not produce potentially toxic free radical as they are metabolized. It is also possible that the early use of a direct dopamine agonist decreases the propensity to develop the late complications, associated with direct stimulation of the dopamine receptor by dopamine itself, such as the “on-off” effect and dyskinesia.

Monoaminoxidase-B inhibitors (MAO) such as selegiline (Diprenyl, or Eldepryl), taken in a low dose, may reduce the progression of Parkinsonism. These compounds can be used as an adjunctive medication. A study has documented that selegiline delays the need for levodopa by roughly three months, although interpretation of this data is confounded by the mild symptomatic benefit of the drug. Nonetheless, theoretical and in vitro support for a neuroprotective effect for some members of the selective MAOB class of inhibitors remains (e.g., rasagiline).

Catechol-O-methyltransferase inhibitors (COMT) can also be used in combination treatments of the invention. Catechol-O-methyltransferase is an enzyme that degrades levodopa, and inhibitors can be used to reduce the rate of degradation. Entacapone is a peripherally acting COMT inhibitor, which can be used in certain methods and compositions of the invention. Tasmar or Tolcapone, approved by the FDA in 1997, can also be used in certain methods and compositions of the invention. Psychiatric adverse effects that are induced or exacerbated by PD medication include psychosis, confusion, agitation, hallucinations, and delusions. These can be treated by decreasing dopaminergic medications, reducing or discontinuing anticholinergics, amantadine, catechol O-methyltransferase inhibitors (COMTIs), or monoamine oxidase inhibitors (MAOIs), or by adding low doses of atypical antipsychotics such as clozapine or quetiapine.

The inventive therapy can also be used in conjunction with surgical therapies for the treatment of PD. Surgical treatment is presently recommended predominantly for those who have failed medical management of PD. Unilateral thallamotomy can be used to reduce tremor. It is occasionally considered for patients with unilateral tremor not responding to medication. Bilateral procedures are typically not advised. Unilateral deep brain stimulation of the thalamus for tremor may also be a benefit for tremor. Unilateral pallidotomy is an effective technique for reducing contralateral drug-induced dyskinesias. Gamma knife surgery—thalamotomy or pallidotomy—can be performed as a radiological alternative to conventional surgery. The currently preferred neurosurgical intervention is, however, bilateral subthalamic nucleus stimulation. Neurotransplantation strategies remain experimental. In addition to surgery and medication, physical therapy in Parkinsonism maintains muscle tone, flexibility, and improves posture and gait.

In other aspects, the inventive therapy can be used in conjunction with one or more other medications for treating DLBD. For example, the lowest acceptable doses of levodopa can be used to treat DLBD. D2-receptor antagonists, particularly traditional neuroleptic agents, can provoke severe sensitivity reactions in DLBD subjects with an increase in mortality of two to three times. Cholinesterase inhibitors discussed herein may also be used in conjunction with the inventive treatment of DLBD. Glutamate antagonists such as memantine may also be used

In treating MSA, the inventive treatment can be used in conjunction with one or more alternative medications for treating the symptoms of MSA. Typically, the drugs that can be used to treat various symptoms of MSA become less effective as the disease progresses. Levodopa and dopamine agonists used to treat PD are sometimes partially effective for the slowness and rigidity of MSA. Orthostatic hypertension can be improved with cortisone, midodrine, fludrocortisone, or other drugs that raise blood pressure. Male impotence may be treated with penile implants or drugs. Incontinence may be treated with medication or catheterization. Constipation may improve with increased dietary fiber or laxatives.

### Farnesyl Transferase Inhibitor

In another aspect, the invention provides methods for treating a subject with a synucleinopathy or other neurodegenerative diseases by administering amounts of a farnesyl transferase inhibitor and a compound of formula I that inhibits the aggregation of α-synuclein, that are therapeutically effective when combined. In certain embodiments, the farnesyl transferase inhibitor has been shown to be useful in the treatment of synucleinopathies or other neurodegenerative diseases. In certain embodiments, the agents (i.e., the farnesyl transferase inhibitor and the α-synuclein aggregation inhibitor) are small molecules. In certain embodiments, the farnesyl transferase inhibitor is of one of the formulae disclosed herein, or a derivative, analog, stereoisomer, isomer, solvate, salt, or other form thereof. Any farnesyl transferase inhibitor known in the art may be combined with an inventive indatraline derivative that inhibits the aggregation of α-synuclein to form an inventive combination for the treatment of a synucleinopathy. In certain embodiments, the farnesyl transferase inhibitor is LNK-754 (OSI-754; CP-609,754). In certain embodiments, the farnesyl transferase inhibitor is Zarnestra. In certain embodiments, the farnesyl transferase inhibitor is SCH66336 (lonafarnib, Sarasar). In certain embodiments, the farnesyl transferase inhibitor is SCH44342. In certain embodiments, the farnesyl transferase inhibitor is Tipifarnab. In certain embodiments, the α-synuclein aggregation inhibitor is indatraline or a derivative, analog, stereoisomer, isomer, solvate, salt, or other form thereof. In certain embodiments, the α-synuclein aggregation inhibitor is a compound of formula I. In certain embodiments, the doses of one or both of the agents are lower than when the agents are used individually. The additive and/or synergistic effect of the inventive combination may be particularly useful in the chronic treatment of a synucleinopathic subject in order to prevent undesired side effects. The agents may be administered together or sequentially.

In certain embodiments, the inventive combination comprises a farnesyl transferase inhibitor of the formula:

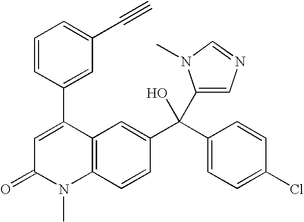

or a pharmaceutically acceptable derivative, analog, stereoisomer, isomer, solvate, salt, or other form thereof. In certain embodiments, the tartrate salt of the compound is used. This compound is also known by the names, LNK-754, OSI-754, and CP-609754.

In certain embodiments, the inventive combination comprises a farnesyl transferase inhibitor of the formula:

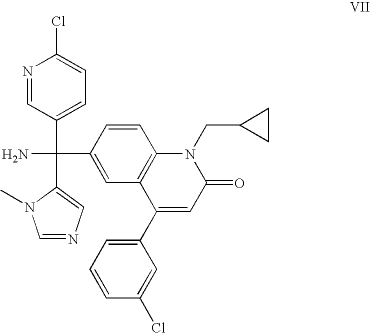

or a pharmaceutically acceptable derivative, analog, stereoisomer, isomer, solvate, salt, or other form thereof. In certain embodiments, the tartrate salt of the compound is used. This compound is also known by the names LNK-427 and OSI-427. In certain particular embodiments, the compound of formula VII useful in accordance with the present invention is (+)-6-[amino-(6-chloro-pyridin-3-yl)-(3-methyl-3H-imidazol-4-yl)-methyl]-4-(3-chloro-phenyl)-1-cyclopropylmethyl-1H-quinoline-2-one (LNK-427). In certain particular embodiments, the compound of formula VII useful in the invention is (−)-6-[amino-(6-chloro-pyridin-3-yl)-(3-methyl-3H-imidazol-4-yl)-methyl]-4-(3-chloro-phenyl)-1-cyclopropylmethyl-1H-quinoline-2-one.

In certain embodiments, the farnesyl transferase inhibitor used in accordance with the present invention is of the formula:

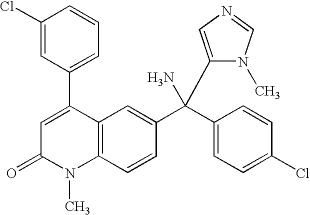

This compound is also known by the name Zarnestra.

In certain embodiments, the farnesyl transferase inhibitor used in accordance with the present invention is of the formula:

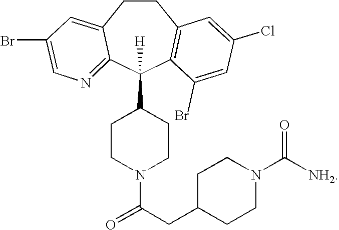

This compound is also known by the name SCH66336 or Sarasar.

The therapeutically effective amount of the farnesyl transferase inhibitor included in the combination therapy will vary depending on the patient, the disease being treated, extent of disease, the route of administration, other medications being administered to the patient, desired outcome, etc. In certain embodiments, the farnesyl transferase inhibitor is administered in the range of approximately 0.0001 mg/kg body weight to approximately 10 mg/kg body weight. In certain embodiments, the farnesyl transferase inhibitor is administered in the range of approximately 0.001 mg/kg body weight to approximately 1 mg/kg body weight. In certain embodiments, the farnesyl transferase inhibitor is administered in the range of approximately 0.001 mg/kg body weight to approximately 0.1 mg/kg body weight. In certain embodiments, the farnesyl transferase inhibitor is administered in the range of approximately 0.01 mg/kg body weight to approximately 0.1 mg/kg body weight. As will be appreciated by one of skill in the art, depending on the form of the farnesyl transferase inhibitor being administered the dosing may vary. The dosages given herein are dose equivalents with respect to the active ingredient. In certain embodiments, the farnesyl transferase inhibitor is administered parenterally. In certain embodiments, the farnesyl transferase inhibitor is administered intravenously. In certain embodiments, the farnesyl transferase inhibitor is administered orally. In certain embodiments, the farnesyl transferase inhibitor is administered from once a week to 2-3 times per day. In certain particular embodiments, the farnesyl transferase inhibitor is administered once per day. In certain embodiments, the farnesyl transferase inhibitor and synuclein aggregation inhibitor are administered together. In other embodiments, they are administered separately. In certain embodiments, the combination is administered long term to prevent the development of the synucleinopathy or other neurodegenerative diseases.

### Pharmaceutical Composition

The present invention also provides pharmaceutical compositions, preparation, and kits comprising a compound of formula I that inhibits the aggregation of α-synuclein, and optionally a pharmaceutically acceptable carrier or excipient. The compositions, preparation, and kits typically include amounts of the inventive compound appropriate for the administration to a subject. A kit may contain the inventive pharmaceutical composition as well as instructions for prescribing the composition.

Any pharmaceutical acceptable carrier or excipient may be part of the inventive pharmaceutical compositions. Wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the inventive compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters, polyacrylates, polyphosphazenes, and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (e.g., capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form.

Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration,” and “administered peripherally” as used herein mean the administration of a compound, drug, combination, pharmaceutical composition, or other material other than directly into the central nervous system, such that it enters the patient's system and, thus, is subject to metabolism and other like processes, for example, subcutaneous administration.

These compounds or compositions may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is provided to a synucleinopathic subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the synucleinopathic subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

The compounds according to the invention may be formulated for administration in any convenient way for use in human or veterinary medicine, by analogy with other pharmaceuticals.

According to the invention, compounds for treating neurological conditions or diseases can be formulated or administered using methods that help the compounds cross the blood-brain barrier (BBB). The vertebrate brain and CNS has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, farnesyl transferase inhibitor compounds that cross the BBB are particularly useful for treating synucleinopathies. In one embodiment, it is expected that farnesyl transferase inhibitors that are non-charged (e.g., not positively charged) and/or non-lipophilic may cross the BBB with higher efficiency than charged (e.g., positively charged) and/or lipophilic compounds. Therefore it will be appreciated by a person of ordinary skill in the art that some of the compounds of the invention might readily cross the BBB. Alternatively, the compounds of the invention can be modified, for example, by the addition of various substitutents that would make them less hydrophilic and allow them to more readily cross the BBB.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697, incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527, incorporated herein in its entirety by reference).

A ligand-neuropharmaceutical agent fusion protein is another method useful for delivery of compositions to a subject (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596, incorporated herein in its entirety by reference), or polypeptides called receptor mediated permeabilizers (RMP) (U.S. Pat. No. 5,268,164, incorporated herein in its entirety by reference). Exogenous molecules can be administered to the subject's bloodstream parenterally by subcutaneous, intravenous, or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g., between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g., bradykinin) can be administered before or at the same time as an intravenous injection of a drug. Thus, the term “co-administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, compounds of the invention can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the inventive compound (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836; and 6,407,137, the disclosures of which are incorporated herein by reference in their entirety.

The administration of the agents of the present invention may be for either prophylactic or therapeutic purposes. When provided prophylactically, the agent is provided in advance of disease symptoms. The prophylactic administration of the agent serves to prevent or reduce the rate of onset of symptoms of a synucleinopathy. When provided therapeutically, the agent is provided at (or shortly after) the onset of the appearance of symptoms of actual disease. In some embodiments, the therapeutic administration of the agent serves to reduce the severity and duration of the disease.

The function and advantage of these and other embodiments of the present invention will be more fully understood from the examples described below. The following examples are intended to illustrate the benefits of the present invention, but do not exemplify the full scope of the invention.

### EXAMPLES

### Example 1

### Preparation of Compound LNK 121 and LNK 122

### Preparation of Compound 1-2a

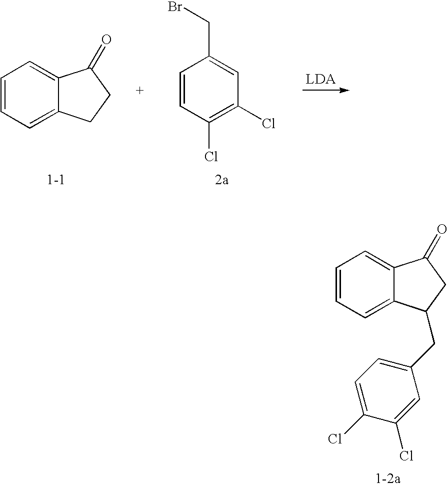

LDA (2 M in THF/Heptane, 11.36 ml, 22.71 mmol, 3.0 equiv.) was cooled to −75° C. under Ar, then compound 1-1 (1.0 g, 7.57 mmol, 1.0 equiv.) in THF (16 ml) was added into it in a period of 20 min. The reaction was stirred at −75° C. for 1 h, then for 2.5 hrs without cooling. Compound 2a (2.36 g, 9.841 mmol, 1.3 equiv.) in THF (4 ml) was added dropwise rapidly at about −10° C., and stirred at −10° C. for 1 h. Then the reaction was quenched with 30 ml of NaCl (Saturated solution), 30 ml of HCl (3N) at −10 ˜−20° C., extracted with Et₂O (3×30 ml), the organic layer combined was washed with Na₂CO₃ (Sat. 2×15 ml), brine (15 ml), dried over Na₂SO₄. The solvent was removed and the residue was purified by flash chromatography (ethyl acetate (EA)/hexanes=5%) to give 0.5 g of compound 1-2a as a pale yellow solid (containing some THF by ¹H-NMR; ¹H-NMR confirmed; yield: 23%).

¹H-NMR (CDCl₃, 400 MHz): δ 7.77 (d, J=7.6 Hz, 1H), 7.61 (t, J=7.6 Hz, 1H), 7.43 (t, J=7.6 Hz, 1H), 7.39 (d, J=8.0 Hz, 1H), 7.37 (d, J=7.6 Hz, 1H), 7.29 (d, J=2.0 Hz, 1H), 7.01 (dd, J=8.0, 2.0 Hz, 1H), 3.74-3.66 (m, 1H), 3.13 (dd, J=14.0, 6.0 Hz, 1H), 2.85-2.76 (m, 2H), 2.40 (dd, J=19.2, 3.2 Hz, 1H).

### Preparation of Compound 1-3a

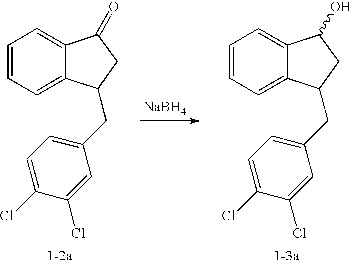

Compound 1-2a (0.76 g, 2.59 mmol, 1.0 equiv.) was suspended in MeOH (8.5 ml), then cooled to 0° C., then NaBH₄ (98 mg, 2.59 mmol) was used at 0° C. The reaction was stirred at RT for 2 hrs, TLC shows the reaction was complete and the solvent was removed, then the residue was treated with H₂O (4 ml), extracted with Et₂O, the organic layer was combined and concentrated, then purified by flash chromatography (ethyl acetate/hexanes=6%) to give 627 mg of pure compound 1-3a as a white solid (¹H-NMR & MS confirmed; yield: 83%, the ratio of cis to trans was about 1 to 0.35)

¹H-NMR (CDCl₃, 400 MHz, cis-isomer): δ 7.45-7.42 (m, 1H), 7.40 (d, J=8.4 Hz, 1H), 7.34 (d, J=2.0 Hz, 1H), 7.32-7.29 (m, 2H), 7.18-7.15 (m, 1H), 7.07 (dd, J=8.4, 2.0 Hz, 1H), 5.18 (dd, J=12.4, 6.4 Hz, 1H), 3.36-3.29 (m, 1H), 3.23 (dd, J=13.6, 6.0 Hz, 1H), 2.77 (dd, J=13.6, 8.8 Hz, 1H), 2.56 (dt, J=13.2, 7.2 Hz, 1H), 1.87-1.82 (m, 1H), 1.67-1.60 (m, 1H).

### Preparation of Compound 1-4a

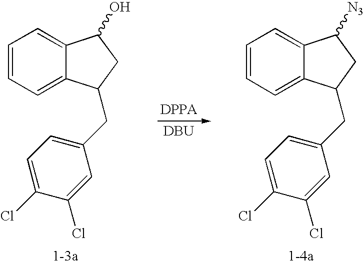

To the solution of 1-3a (626 mg, 2.14 mmol, 1.0 equiv.) in THF (12 ml) was added DPPA (822 mg, 2.99 mmol, 1.4 equiv.), the mixture was stirred for 10 min and then cooled to 0° C., DBU (455 mg, 2.99 mmol, 1.4 equiv.) was added into the reaction via syringe and the mixture was warmed to RT and stirred for two days, TLC shows little compound 1-3a was left and one main new spot appeared. The solvent was removed and the residue was purified directly by flash chromatography (EA/Hex=1:100) to give 404 mg of compound 1-4a as a colorless oil. (¹H-NMR & MS confirmed; yield: 59%, trans/cis=˜1/0.35 by ¹H-NMR).

¹H-NMR (CDCl₃, 400 MHz, trans-isomer): δ 7.44-7.30 (m, 5H), 7.18 (d, J=7.2 Hz, 1H), 7.02 (dd, J=8.0, 2.0 Hz, 1H), 4.84-4.81 (m, 1H), 3.70-3.62 (m, 1H), 3.12 (dd, J=14.0, 6.0 Hz, 1H), 2.67 (dd, J=14.0, 8.8 Hz, 1H), 2.21 (ddd, J=13.6, 7.2, 4.5 Hz, 1H), 2.09 (dt, J=13.6, 7.2 Hz, 1H).

Preparation of Compound 1-5a′

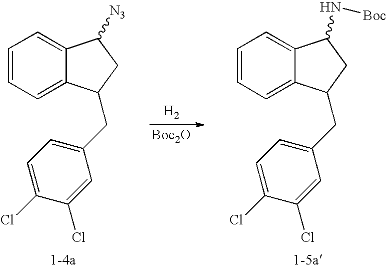

To the mixture of Pd/C (20 mg, 7.5%) in EA (5 ml) was added the solution of compound 1-4a (0.4 g, 1.26 mmol, 1.4 equiv.) and Boc₂O (0.38 g, 1.76 mmol, 1.4 equiv.) in EtOAc (5 mL) at RT, then the mixture was hydrogenation with a H₂ balloon for 2 hrs. TLC shows some compound 1-4a was left and the reaction was stirred overnight. TLC shows the reaction completed. The mixture was purified directly by flash chromatography (EA/PE=1:10) to give 360 mg of compound 1-5a′ as a colorless oil (¹H-NMR & MS confirmed; yield: 73%)

¹H-NMR (CDCl₃, 400 MHz, trans-isomer): δ 7.41-7.19 (m, 5H), 7.03-6.96 (m, 2H), 5.26-5.19 (m, 1H), 4.69-4.64 (m, 1H), 3.54-3.48 (m, 1H), 2.92 (dd, J=13.6, 6.8 Hz, 1H), 2.67 (dd, J=13.6, 8.8 Hz, 1H), 2.29 (m, 1H), 1.96 (m, 1H), 1.49 (s, 9H).

### Preparation of Compound 1-5a

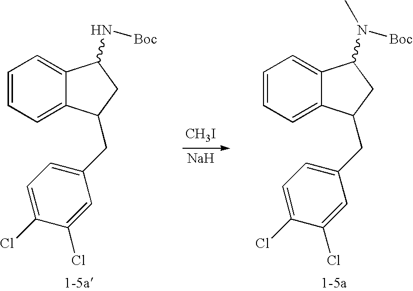

To the solution of 1-5a′ (0.36 g, 0.918 mmol, 1.0 equiv.) in DMF (5.4 ml) was added NaH (0.116 g, 4.59 mmol, 5.0 equiv.) at about −10° C., the mixture was stirred for 2.5 hrs and then cooled to −10° C., CH₃I (0.81 ml) was added into the reaction mixture via syringe and the mixture was allowed to RT overnight. TLC shows reaction completed. The reaction was quenched with water (5 ml), extracted with Et₂O, then purified by flash chromatography (EA/Hex=1:15) to give 323 mg of compound 1-5a as a yellow oil. (Mass confirmed; yield: 87%). MS (ESI⁺) 428.3 (M+23)

Preparation of Compound LNK-121 HCl salt

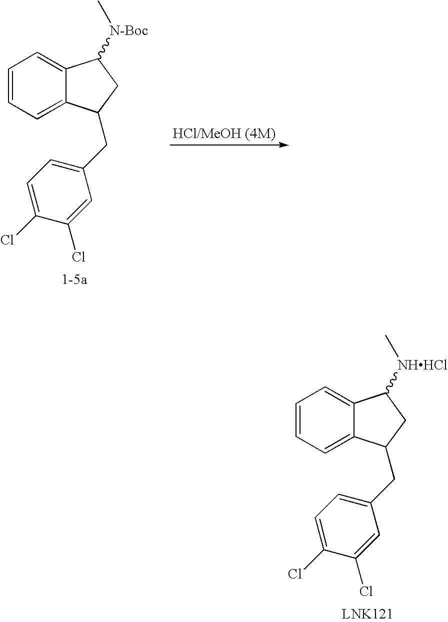

Compound 1-5a (60 mg, 0.148 mmol) was dissolved in HCl-MeOH (4 M) and stirred for 2 hrs at RT, TLC shows the reaction completed, and the result was concentrated to give 52 mg of compound LNK-121 HCl salt as a white solid. (¹H-NMR (CD₃OD) & MS confirmed; yield: 93%) HPLC: (96%˜97%) MS: (306.2, +Q1) trans/cis: 1.00/0.32

¹H-NMR (CD₃OD, 400 MHz, trans-isomer): δ 7.55-7.36 (m, 6H), 7.18 (dd, J=8.0, 2.0 Hz, 1H), 4.65 (dd, J=6.4, 4.4 Hz, 1H), 3.80-3.73 (m, 1H), 3.24 (dd, J=13.6, 6.4 Hz, 1H), 3.72-3.66 (m, 1H), 2.68 (s, 3H), 2.28-2.23 (m, 2H).

### Preparation of Compound LNK 122 HCl Salt

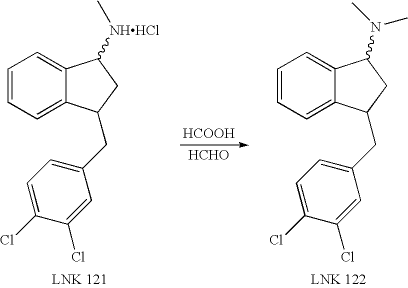

To 68 mg of compound LNK-121 HCl salt (0.199 mmol) was added HCOOH/HCHO (5:1, 0.43 ml), and the mixture was refluxed for 3 h, then the reaction mixture was neutralized by NH₄OH (28%), extracted with DCM, washed with brine, dried over Na₂SO₄, concentrated to give 55 mg of crude product. The crude product was suspended in Et₂O, then HCl-Et₂O was added and stirred for 30 min, then concentrated to dry and dissolved in DCM, the white solid which did not dissolved in DCM was filtered off, The DCM solution was dried and triturated with DCM/Et₂O (about 5:1), filtered to give 8 mg of LNK-122 HCl salt as a white solid which is hygroscopic (¹H-NMR & MS confirmed; HPLC: 90.5%; MS: 320,322.1, +Q1; trans/cis=1.00/0.35).

¹H-NMR (CDCl₃, 400 MHz, trans-isomer): δ 12.73 (brs, 1H), 7.94 (d, J=6.0 Hz, 1H), 7.49-7.28 (m, 4H), 7.26 (d, J=7.2 Hz, 1H), 7.03 (d, J=6.8 Hz, 1H), 4.89-4.85 (m, 1H), 3.77-3.73 (m, 1H), 3.18-3.13 (m, 1H), 2.87-2.49 (m, 2H), 2.69 (s, 3H), 2.62 (s, 3H), 2.22-2.17 (m, 1H). MS (284.3, M+1). HPLC: 98.3% (Column: Phenomenex Luna 5μC18(2) 150×4.6 mm, Retention Time: 1.588 min. Mobile phase: methanol/buffer (0.01% TFA)=65/35, Wavelength: 254 nm, Flow rate: 1.0 ml/min.).

### Example 2

### Preparation of Compound LNK 130

### Preparation of Compound 1-2b.

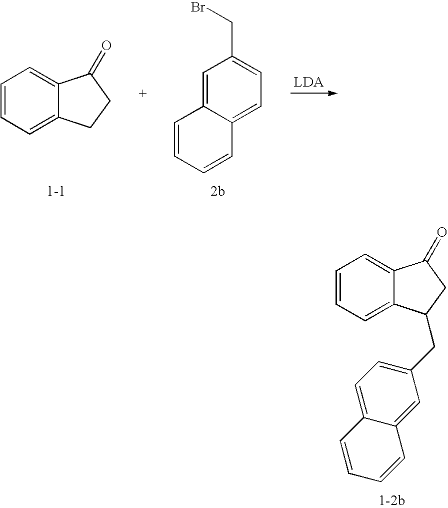

LDA (19 ml, 37.8 mmol, 2.5 equiv.) was cooled to −75° C. under Ar, then compound 1-1 (2.0 g, 15.1 mmol, 1.0 equiv.) in THF (32 ml) was added into it in a period of 30 min. The reaction was stirred at −75° C. for 1 h, then for 2.5 hrs without cooling. Compound 2b (4.18 g, 18.9 mmol, 1.25 equiv.) in THF (8 ml) was added dropwise rapidly at about −15° C., then kept at about 0° C. for 2.5 h. Then the result was quenched with 64 ml of NaCl (Sat.), 64 ml of HCl (3 N) at −10˜−20° C., extracted with Et₂O (3×80 ml), washed with Na₂CO₃ (Sat. 2×30 ml), dried over Na₂SO₄. The mixture was purified by flash chromatography (ethyl acetate/hexanes=5%) to give a mixture of the desired product and compound 1-1, which was recrystallized to give 0.723 g of pure compound 1-2b (¹H-NMR & MS confirmed; yield: 17.6%) as a pale white solid.

¹H-NMR (CDCl₃, 400 MHz): δ 8.16 (d, J=8.4 Hz, 1H), 7.94 (d, J=8.0 Hz, 1H), 7.82 (dd, J=8.4, 7.6 Hz, 1H), 7.64-7.53 (m, 3H), 7.48-7.40 (m, 3H), 7.32 (d, J=7.2 Hz, 1H), 3.98-3.91 (m, 1H), 3.73 (dd, J=14.0, 5.6 Hz, 1H), 3.17 (dd, J=14.4, 9.6 Hz, 1H), 2.79 (dd, J=15.2, 7.6 Hz, 1H), 2.58 (dd, J=15.2, 3.2 Hz, 1H).

### Preparation of Compound 1-3b.

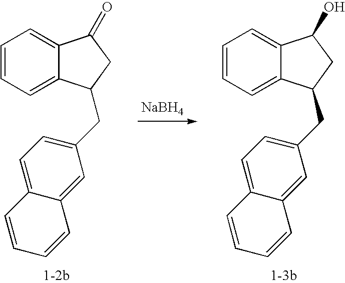

Compound 1-2b (0.3 g, 1.10 mmol, 1.0 equiv.) was suspended in MeOH (3.6 ml) and cooled to 0° C., then NaBH₄ (41.4 mg, 1.1 mmol, 1.0 equiv.) was added at 0° C. The reaction was stirred at RT overnight. The precipitate formed was collected and dissolved in Et₂O, washed with H₂O (6 ml), then was directly purified by flash chromatography (EA/PE=6%) to give 213 mg of pure compound 1-3b was obtained as a white solid (yield: 71%).

¹H-NMR (CDCl₃, 400 MHz, cis isomer): δ 8.17 (d, J=8.4 Hz, 1H), 7.92 (d, J=8.0 Hz, 1H), 7.79 (d, J=8.0 Hz, 1H), 7.57-7.50 (m, 2H), 7.48-7.37 (m, 3H), 7.35-7.29 (m, 3H), 5.15 (dd, J=12.0, 6.4 Hz, 1H), 3.87 (dd, J=14.0, 5.6 Hz, 1H), 3.61-3.48 (m, 1H), 3.16 (dd, J=13.6, 9.6 Hz, 1H), 2.49 (dt, J=13.6, 7.2 Hz, 1H) 1.82 (brs, 1H), 1.77 (m, 1H).

### Preparation of Compound 1-4b.

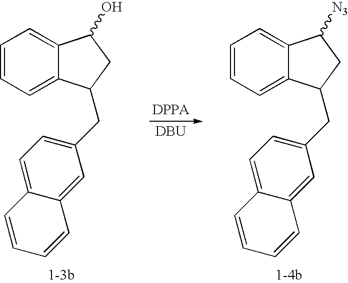

To the solution of 1-3b (155 mg, 0.566 mmol, 1.0 equiv.) in THF (3 ml) was added DPPA (218 mg, 0.792 mmol, 1.4 equiv.), the mixture was stirred for 10 min and then cooled to 0° C., DBU (120 mg, 0.792 mmol, 1.4 equiv.) was added into the reaction via syringe and the mixture was allowed to RT for two days, TLC shows a lot of compound 1-3b was left and one main new spot appeared. Another 135 mg of DPPA and 75 mg of DBU were added and stirred overnight. TLC shows still some compound 1-3b was left. The mixture was purified directly by flash chromatography (EA/Hex=1:100) to give 105 mg of compound 1-4b as a colorless oil. (yield: 65%) and 42 mg of S.M. 1-3b as a white solid was recovered.

¹H-NMR (CDCl₃, 400 MHz, trans isomer): δ 8.12 (d, J=8.0 Hz, 1H), 7.92 (d, J=8.0 Hz, 1H), 7.80 (d, J=8.4 Hz, 1H), 7.58-7.50 (m, 2H), 7.46-7.40 (m, 2H), 7.38-7.29 (m, 4H), 4.94 (dd, J=6.4, 4.0 Hz, 1H), 3.89 (dd, J=14.0, 6.4 Hz, 1H), 3.71 (dd, J=14.0, 6.4 Hz, 1H), 3.05 (dd, J=14.0, 9.6 Hz, 1H), 2.26-2.11 (m, 2H).

Preparation of Compound 1-5b′.

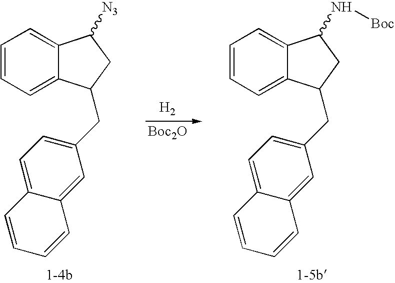

To the mixture of Pd/C (6.5 mg) in EA (1.5 ml) was added the solution of compound 1-4b (123 mg, 0.411 mmol, 1.0 equiv.) and Boc₂O (126 mg, 0.576 mmol, 1.4 equiv.) in EA (2 ml) at RT, the mixture was hydrogenated with a H₂ balloon overnight. TLC shows the reaction completed. The mixture was purified directly by flash chromatography (EA/Hex=1:15) to give 59 mg of compound 1-5b′ as a colorless oil (¹H-NMR (CDCl₃) & MS (396.3, +Q1) confirmed; iso-ratio: 1.00/0.25; yield: 39%).

¹H-NMR (CDCl₃, 400 MHz, trans isomer): δ 8.12 (d, J=7.6 Hz, 1H), 7.91 (d, J=7.6 Hz, 1H), 7.78 (d, J=8.0 Hz, 1H), 7.58-7.50 (m, 2H), 7.46-7.21 (m, 5H), 7.07 (d, J=6.8 Hz, 1H), 5.41-5.36 (m, 1H), 4.71-4.67 (m, 1H), 3.73-3.69 (m, 1H), 3.51 (dd, J=13.6, 9.6 Hz, 1H), 3.09 (dd, J=13.6, 9.6 Hz, 1H), 2.44-2.39 (m, 1H), 1.91 (dt, J=13.6, 6.8 Hz, 1H), 1.48 (s, 9H).

### Preparation of Compound 1-5b.

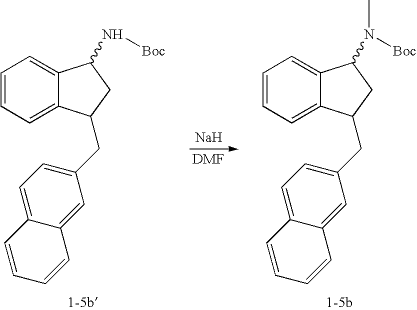

To the solution of 1-5b′ (80 mg, 0.214 mmol, 1.0 equiv.) in DMF (1.26 ml) was added NaH (26 mg, 1.07 mmol, 5.0 equiv.) at about −10° C., the mixture was stirred for 2.5 hrs and then cooled to −10° C., CH₃I (0.19 ml) was added into the reaction via syringe and the mixture was then stirred at RT overnight. TLC shows the reaction completed. The reaction was quenched with water (5 ml), extracted with Et₂O, then purified by flash chromatography (ethyl acetate/hexanes=1:15) to give 72 mg of compound 1-5b as a yellow oil (¹H-NMR (DMSO-d6) & MS (388.3, +Q1) confirmed; trans/cis: 1.00/0.28; yield: 87%)

¹H-NMR (DMSO-d6, 400 MHz, trans isomer): δ 8.20 (d, J=8.4 Hz, 1H), 7.95 (d, J=7.6 Hz, 1H), 7.82 (d, J=8.0 Hz, 1H), 7.60-7.43 (m, 4H), 7.34 (d, J=6.4 Hz, 1H), 7.31-7.18 (m, 2H), 7.11 (d, J=6.4 Hz, 1H), 3.72-3.65 (m, 1H), 3.49 (dd, J=13.6, 5.2 Hz, 1H), 3.27 (m, 1H), 3.04 (dd, J=13.6, 9.6 Hz, 1H), 2.72 (s, 3H), 2.13-1.93 (m, 2H), 1.41 (s, 9H).

### Preparation of Compound LNK 130.

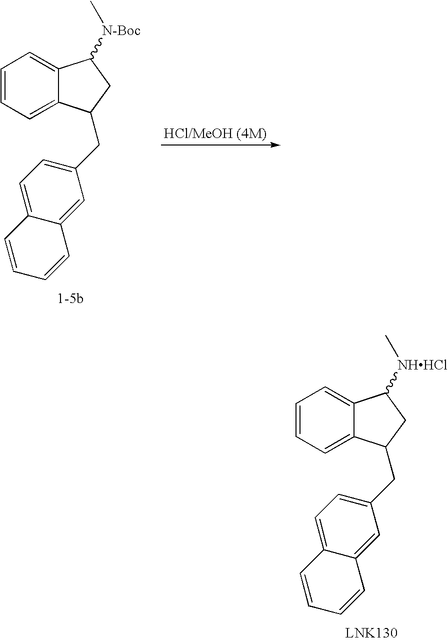

Compound 1-5b (59 mg, 0.152 mmol, 1.0 equiv.) was dissolved in HCl-MeOH (4 M, 1 ml) and stirred for 2.5 hrs at RT, TLC shows the reaction completed and the reaction was concentrated to give 48 mg of compound LNK 130 as a pale yellow solid (¹H-NMR (CD₃OD) & MS confirmed; yield: 98%). HPLC: (100%) MS: (288, +Q1) trans/cis: 1.00/0.27

¹H-NMR (CD₃OD, 400 MHz, trans isomer): δ 8.22 (d, J=9.2 Hz, 1H), 7.93 (d, J=8.0 Hz, 1H), 7.82 (d, J=8.0 Hz, 1H), 7.60-7.38 (m, 8H), 4.78 (dd, J=7.2, 3.2 Hz, 1H), 4.01-4.96 (m, 1H), 3.85 (dd, J=14.0, 5.6 Hz, 1H), 3.06 (dd, J=14.0, 9.6 Hz, 1H), 2.67 (s, 3H), 2.39 (dt, J=14.4, 7.2, 1H), 2.16 (ddd, J=14.0, 7.2, 3.2 Hz, 1H).

### Example 3

### Preparation of Compound LNK-131

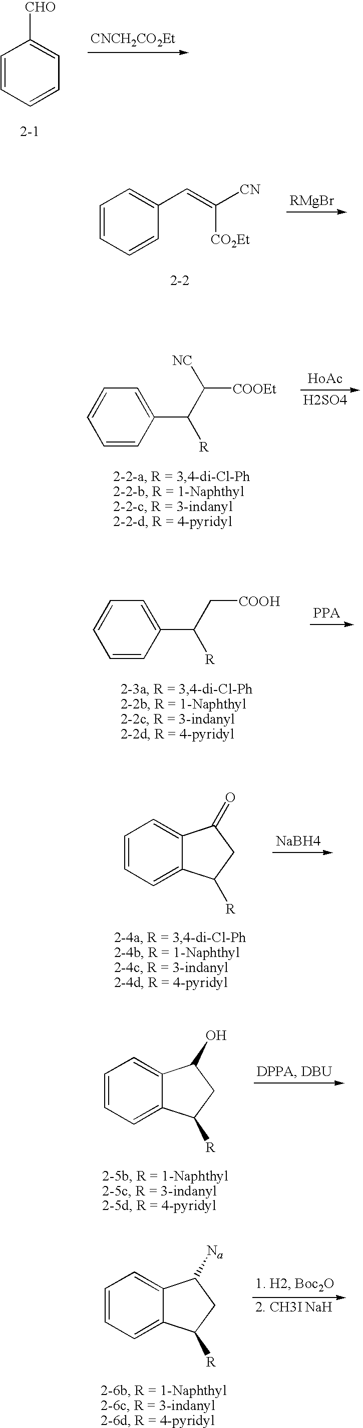

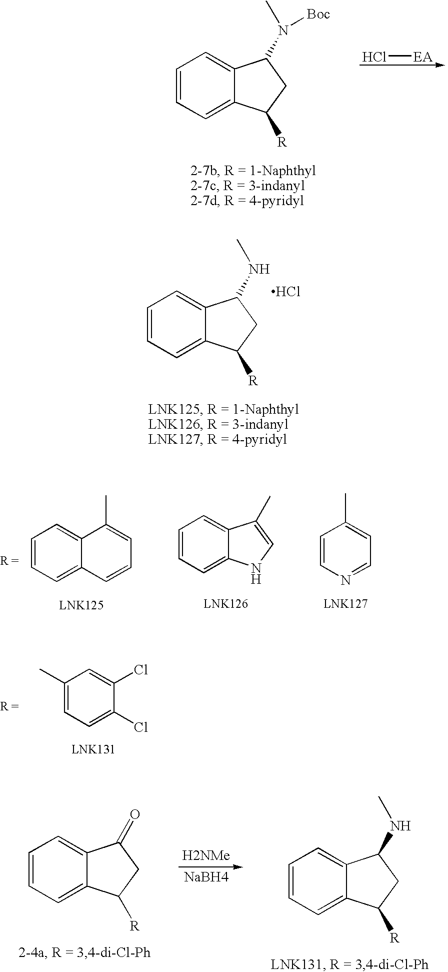

Preparation of compound 2-2.

To a 100 mL flask charged with compound 2-1 (10 g, 94.2 mmol) was added 30 mL toluene, CNCH₂COOEt (12 g, 106 mmol, 1.13 equiv.). Piperidine (0.06 mL) was added. The mixture was refluxed for 30 min with a Dean-Stark apparatus. Nearly 2 mL of water was separated, TLC (EA:PE=1:6) showed reaction complete.

Solvent was removed under vacuum. The obtained residue was taken into 60 mL mix solvent of isopropyl ether-hexane (1:1), stand in refrigerator for 2 hrs. A light yellow crystal formed, filtered and dried to give a light yellow crystal of compound 2-2 (17.32 g, ¹H-NMR confirmed, 91.3% yield)

¹H-NMR (CDCl₃, 400 MHz): δ 8.27 (s, 1H), 8.10-7.90 (m, 2H), 7.65-7.45 (m, 3H), 4.41 (d, 2H, J=7.2 Hz), 1.42 (t, 3H, J=7.2 Hz).

### Preparation of Compound 2-2a.

To a 50 mL flask charged with the (3,4-dichlorophenyl)magnesium bromide (22.8 mL, 11.4 mmol, 1.14 equiv.) was added compound 2-2 (2.0 g, 10 mmol, 1 equiv.) in 10 mL extra dry THF by a syringe under argon. The obtained clear yellow solution was stirred at reflux for 30 min. TLC showed reaction complete. 15 mL toluene was added, THF was removed under vacuum. The reaction solution was poured into a mixture of 25 mL ice and 2.5 mL con. H₂SO₄, separated, the water layer was extracted with toluene 20 mL×2, organics was combined and dried over Na₂SO₄, filtered and concentrated to get compound 2-2-a as a light yellow oil (2.34 g, 64.3% yield, ¹H-NMR confirmed).

¹H-NMR (CDCl₃, 400 MHz): δ 7.50-7.10 (m, 7H), 4.76-4.68 (m, 1H), 4.24-4.12 (m, 3H), 1.21-1.12 (m, 3H).

### Preparation of Compound 2-3a.

To a flask charged with compound 2-2-a (520 mg, 1.76 mmol) was added 5 mL HOAc, 2.5 mL concentrated H₂SO₄ and 2.5 mL H₂O. The mixture was stirred at reflux for 24 hrs. The reaction mixture was poured into 25 mL ice, filtered to give compound 2-3a as a brown solid (292 mg, ¹H NMR confirmed).

¹H-NMR (CDCl₃, 400 MHz): δ 7.40-7.29 (m, 4H), 7.26-7.18 (m, 3H), 7.09 (dd, 1H, J=2.0, 8.4 Hz), 4.50 (t, 1H, J=7.6 Hz), 3.08 (d, 2H, J=7.6 Hz).

### Preparation of Compound 2-4a.

To a 25 mL flask was added 8.6 g PPA under argon. Then the PPA was heated at 90° C. Compound 2-3a (180 mg, 0.61 mmol) was added at 90° C. under argon. The obtained mixture was stirred at 110° C. for 4 hrs. TLC showed reaction complete. 25 mL ice-water was added, then extracted with EA 15 mL×4, organic was combined and dried over Na₂SO₄, filtered. The filtrate was concentrated to give a brown oil (140 mg), separated by flash chromatography (DCM:MeOH=20:1) to give a orange solid (29 mg) and desired compound 2-4a as a yellow solid (28 mg, 16.7% yield, ¹H-NMR confirmed).

¹H-NMR (CDCl₃, 400 MHz): δ 7.84 (d, 1H, J=7.6 Hz), 7.61 (t, 1H, J=7.6 Hz), 7.46 (t, 1H, J=7.6 Hz), 7.39 (d, 1H, J=8.4 Hz), 7.30-7.23 (m, 2H), 6.96 (dd, 1H, J=2.0, 8.4 Hz), 4.56 (dd, 1H, J=3.6, 8.0 Hz), 3.25 (dd, 1H, J=8.0, 19.2 Hz), 2.65 (dd, 1H, J=3.6, 8.0 Hz).

### Preparation of Compound LNK-131 HCl Salt.

To a 25 mL flask charged with compound 2-4a (25 mg, 0.09 mmol) was added 3 mL MeOH and several drops of the CH₃NH₂/methanol (27-32%). The mixture was stirred at room temp for 2.5 hours. TLC (EA:PE=1:6) showed reaction complete. 5 mg NaBH₄ was added into the reaction. The mixture was stirred at room temp overnight. TLC showed reaction complete. Solvent was removed. The residue was taken into 4 mL EA, washed with water, separated. Organics was dried over Na₂SO₄, filtered, concentrated to get a yellow oil. The oil was purified by flash chromatography (EA, then EA:MeOH=1:1) to give compound LNK131 as a yellow oil (18 mg, ¹H-NMR confirmed, 69% yield)

2 mL HCl saturated ether was to the above oil, concentrated to afford compound LNK131 hydrochloride as a white solid (12 mg, ¹H-NMR confirmed to be cis-isomer)

¹H-NMR (CDCl₃, 400 MHz): δ 10.3 (brs, 1H), 8.06 (s, 1H), 7.44-7.39 (m, 2H), 7.36-7.30 (m, 2H), 7.14 (d, 1H, J=8.0 Hz), 6.94 (d, 1H, J=7.2 Hz), 4.90-4.80 (m, 1H), 4.35-4.25 (m, 1H), 3.12-3.00 (m, 1H), 2.70-2.63 (s, 3H), 2.40-2.25 (m, 1H). MS (ESI, positive)=291.9 HPLC: 92%

### Example 4

### Preparation of Compound LNK-125

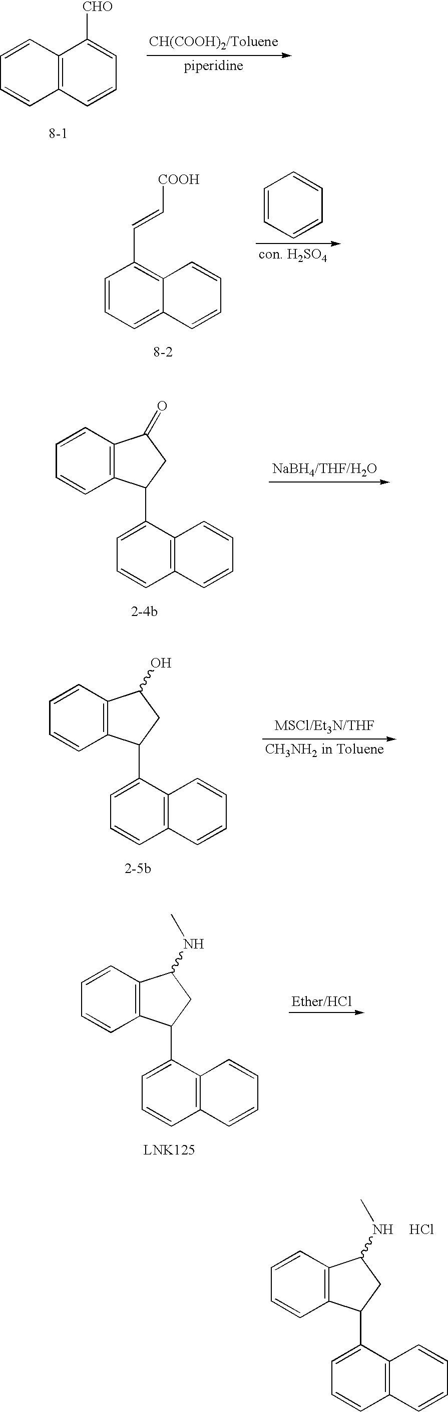

### Preparation of Compound 8-2

To a 10 mL pyridine solution of compound 8-1 (5.0 g, 32 mmol) was added malonic acid (3.5 g, 33 mmol, 1.04 equiv.) and 0.2 mL piperidine. The mixture was stirred at 80° C. for 1 hrs, TLC showed reaction not complete. Then the reaction was stirred at 100° C. for 30 mins and refluxed for 1 hr. TLC showed reaction complete. The reaction was cooled to room temp and poured slowly into 100 mL ice. A precipitate crashed out. Then it was acidified by 6 N HCl, filtered. The obtained white solid (¹H-NMR showed it was a mixture of compound 8-2 and compound 8-1) was recrystallized with EtOH-H₂O to give the desired compound 8-2 as a white solid (3.36 g, 53% yield, ¹H-NMR confirmed).

¹H-NMR (DMSO-d6, 400 MHz): δ 13.0-12.0 (br, 1H), 8.39 (d, 1H, J=16.0 Hz), 8.20 (d, 1H, J=8.4 Hz), 8.05-7.92 (m, 3H), 7.67-7.53 (m, 3H), 6.60 (d, 1H, J=16.0 Hz).

### Preparation of Compound 2-4b

To a flask charged with compound 8-2 (1.0 g, 5 mmol) was added 10 mL benzene and 3.3 mL con. H₂SO₄. The mixture was stirred at 85-95° C. (bath temp) for 30 mins. TLC showed no starting material remained. The reaction was cooled to RT and was poured into 100 mL ice. The mixture was stirred for 30 min, separated. The aqueous layer was extracted with EA (50 mL×3). The organic combined and dried over Na₂SO₄, filtered and concentrated to give a dark residue. Purified by flash chromatography gave the desired product compound 2-4b as a light yellow solid (20 mg, TLC confirmed).

¹H-NMR from pilot reaction (CDCl₃, 400 MHz): δ 7.98-7.90 (m, 2H), 7.85 (d, 1H, J=8.4 Hz), 7.75 (d, 1H, J=8.4 Hz), 7.60 (t, 1H, J=7.2 Hz), 7.42 (t, 1H, J=7.2 Hz), 7.32-7.22 (m, 3H), 7.14-7.10 (m, 2H), 5.04 (dd, 1H, J=2.0, 8.0 Hz), 3.40 (dd, 1H, J=7.6, 18.8 Hz), 2.73 (dd, 1H, J=2.0, 18.8 Hz).

### Preparation of Compound 2-5b

To a 1.5 mL THF solution of the compound 2-4b (38 mg, 0.05 mmol) was added NaBH₄ (6 mg, 0.15 mmol) below 10° C. The mixture was stirred with a ice bath overnight, TLC showed reaction not complete. Another 6 mg NaBH₄ was added, reaction stirred at RT for 2 hrs. TLC showed reaction still not complete. Then 0.15 mL water was added, the reaction was stirred at r.t. 30 min. TLC showed reaction complete. Solvent was removed, the residue was treated with 2 mL EA and 2 mL water, separated and the aqueous layer was extracted with ethyl acetate (2 mL×2). Organic was combined and dried over Na₂SO₄, filtered and concentrated to give compound 2-5b as a pale yellow solid (40 mg, ¹H-NMR showed cis/trans=3:1, 100% yield)

¹H-NMR (CDCl₃, 400 MHz): δ 7.92-7.85 (m, 2.39H), 7.68-7.64 (d, 1H, J=8.4 Hz), 7.50-7.40 (m, 2.25H), 7.37-7.32 (m, 0.43H), 7.32-7.24 (m, 5.47H, contains CDCl₃ peak), 7.24-7.18 (m, 3.12H), 7.07-7.03 (m, 0.65H), 5.68-5.60 (m, 0.37H), 5.46-5.39 (m, 1H), 5.06-5.01 (dd, 0.37H, J=4.0, 7.6 Hz), 4.79-4.72 (dd, 1H, J=5.2, 8.8 Hz), 3.27-3.17 (m, 1H), 2.68-2.62 (m, 0.62H), 2.14-2.06 (m, 1H).

### Preparation of Compound LNK-125 HCl Salt

To a 1.0 ml extra dry THF solution of compound 2-5b (40 mg, 0.15 mmol) under argon was added 0.2 mL Et₃N drop-wise below −15° C. The reaction was cooled to −60° C. MsCl (59 mg dissolved in 250 uL extra dry THF was added drop-wise whilst the reaction temp was kept below −60° C. After the addition, the reaction temp was rose to 0° C. and stirred for 2 hrs at 0° C. Then 4 mL CH₃NH₂ solution in toluene was added and stirred overnight. TLC showed reaction complete. Solvent was removed. The obtained residue was taken into 10 mL EA and 10 mL H₂O, separated; the aqueous layer was extracted with EA (10 mL×2). Organic was combined and washed with brine (10 mL×2), dried over Na₂SO₄, filtered, concentrate to give a residue. 2 mL Ether-HCl solution was added with a ice-water bath, stirred for 30 mins. Then solvent was removed under vacuum. 2 mL ethyl acetate was added, stirred for 30 mins, filtered to give compound LNK125 hydrochloride as a grey solid (13 mg, ¹H NMR confirmed it was the desired product and the ratio of the isomer trans:cis≈2:1). MS (ESI, positive)=274.1, HPLC 99% purity.

¹H-NMR (CD₃OD, 400 MHz): δ 8.02-7.92 (m, 3.12H), 7.75-7.67 (m, 1.62H, 7.55-7.43 (m, 2.73H), 7.38-7.18 (m, 7.82H), 7.10-7.04 (d, 2H, J=7.2 Hz), 5.20-5.11 (m, 2H), 5.05-4.98 (t, 0.58H, J=7.6 Hz), 4.96-4.88 (t, 0.76H, J=8.0 Hz), 3.42-3.32 (m, 0.70H), 2.95-2.86 (m, 1H), 2.81-2.75 (d, 4.41H), 2.66-2.57 (m, 1H), 2.15-2.05 (m, 0.57).

### Example 5

### Preparation of Compound LNK-126

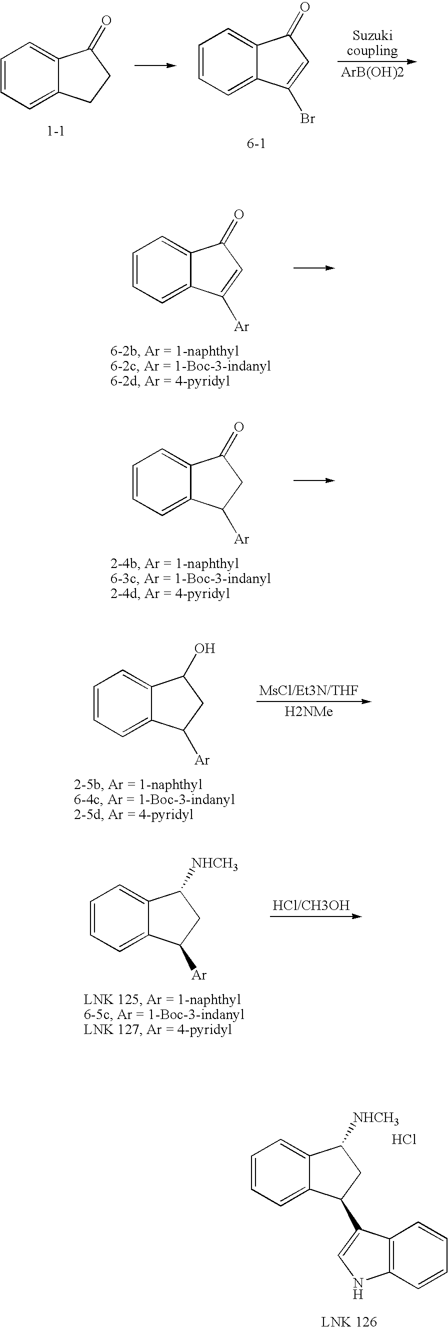

### Preparation of Compound 6-1

To the solution of compound 1-1 (1.32 g, 10 mmol) in 30 ml CCl4 was added NBS (3.74 g, 21 mmol) and 0.1 g of AIBN. The mixture was heated to reflux for overnight. 4.85 ml Et₃N was added at 0° C. and then the mixture was stirred for 12 h at RT. TLC showed a new spot formed. 15 ml of saturated aqueous NaHCO₃ was added and was extracted with ethyl acetate (20 ml×3), dried over Na₂SO₄ and concentrated. Purification by column chromatography (PE) gave compound 6-1 as yellow solid (1.4 g, 67% yield, ¹H-NMR confirmed).

¹H-NMR (DMSO-d6, 400 MHz): δ 7.60 (dt, 1H, J=7.2, 2.0 Hz), 7.45-7.42 (m, 2H), 7.27 (d, 1H, J=7.2 Hz), 6.54 (s, 1H).

### Preparation of Boronic Acid (Compound 6-1c).

To a flask charged with 1-Boc-3-bromoindole (2.0 g, 6.7 mmol) in 10 mL extra dry THF solution was added triethoxyborate (1.46 g, 10.0 mmol, 1.5 equiv.) below −60° C. under argon. The mixture was cooled under −70° C. Then 3.2 mL n-butyllithium (2.5 M in hexane, 8.0 mmol, 1.2 equiv.) was added drop-wise whilst the reaction was kept below −70° C. After the addition the reaction was allowed to warm to the RT slowly within 1 hr. 2 g ice was added to quench the reaction. Solvent was removed under argon. The residue was treated with 25 mL EA and 25 mL water, separated and the aqueous layer was extracted with EA (25 mL×2). Organic was combined and dried over Na₂SO₄, filtered and concentrated. The obtained residue was purified by flash chromatography (EA:PE=1:10) to give a purple solid (297 mg, and a green solid (303 mg). ¹H-NMR showed both of the solid are the desired product compound 6-1c, 34.3% yield.

¹H-NMR (DMSO, 400 MHz): δ 8.17 (s, 1H), 8.07 (s, 2H), 8.03 (d, 1H, J=8.0 Hz), 7.99 (d, 1H, J=6.8 Hz), 7.27 (dd, 1H, J=8.0, 7.2 Hz), 7.22 (d, 1H, J=7.2, 6.8 Hz), 1.64 (s, 9H).

### Preparation of Compound 6-2c.

To a flask charged with the boronic acid (compound 6-1c, 600 mg, 2.3 mmol, 1.1 equiv.) and compound 6-1 (437 mg, 2.1 mmol, 1 equiv.) was added 2 mL EtOH, 2 mL H₂O and 10 mL THF under argon. The mixture was stirred at room temp for 20 min under argon. Then Na₂CO₃ (668 mg, 6.3 mmol, 3 equiv.) and Pd(PPh₃)₄ (121 mg, 0.05 equiv.) was added under argon. The mixture was stirred at room temp under argon over weekend (about 2 days). TLC showed reaction complete. THF was removed under vacuum, the residue was taken into 25 mL DCM, washed with water (20 mL×2), the aqueous layer was extracted with DCM (20 mL×2). Organic was combined and dried over Na₂SO₄, filtered and concentrated, purified by flash chromatography (EA:PE=1:20) to give the desired product as an orange solid (404 mg, ¹H-NMR confirmed, 55.7% yield).

¹H-NMR (CDCl₃, 400 MHz): δ 8.24 (d, 1H, J=7.6 Hz), 8.23 (s, 1H), 7.84 (d, 1H, J=7.6 Hz), 7.59 (d, 1H, J=7.2 Hz), 7.55-7.35 (m, 6H), 6.33 (s, 1H), 1.75 (s, 9H).

### Preparation of Compound 6-3c.

To a flask charged with compound 6-2c (400 mg, 1.16 mmol) was added 2.5 mL THF and 2.5 mL t-BuOH. (PPh₃)₃RhCl (54 mg, 0.06 mmol, 0.05 equiv.) was added under argon. Then the reaction solution was saturated with H₂ and was hydrogenated with a H₂ Balloon at room temp overnight. TLC (EA:PE=1:10) showed 6-2c still remained.

Another 54 mg of (PPh₃)₃RhCl was added, and the reaction was hydrogenated with a H₂ balloon overnight. TLC showed no starting material exist. Solvent was removed to give a dark residue. The residue purified by flash chromatography (EA:PE=1:50) to give the desired compound as a yellow solid (194 mg, ¹H-NMR confirmed, 48% yield).

¹H-NMR (CDCl₃, 400 MHz): δ 8.15 (d, 1H, J=8.0 Hz), 7.88 (d, 1H, J=8.4 Hz), 7.61 (t, 1H, J=7.6 Hz), 7.51-7.45 (m, 2H), 7.40 (s, 1H), 7.36-7.30 (m, 1H), 7.20-7.15 (m, 2H), 4.83 (dd, 1H, J=4.0, 8.0 Hz), 3.8 (dd, 1H, J=8.0, 19.2 Hz), 2.85 (dd, 1H, J=4.0, 19.2 Hz), 1.68 (s, 9H).

### Preparation of Compound 6-4c.

To a 1.5 mL THF solution of compound 6-3c (100 mg, 0.29 mmol) was added 0.15 mL water. The mixture was cooled below −10° C. NaBH₄ (11 mg, 0.29 mmol) was added. The reaction temp was allowed to warm to RT within 30 min and stirred at room temp for 2 hrs. TLC showed reaction complete. THF was removed under vacuum. The obtained residue was taken into 10 mL EA and 10 mL H₂O, separated, the aqueous layer was extracted with 10 mL ethyl acetate. Organics were combined, dried over Na₂SO₄, and filtered. The filtrate was concentrated to afford the desired compound 6-4c as a white solid (99 mg, ¹H-NMR confirmed the major product is cis isomer, 98% yield).

¹H-NMR (CDCl₃, 400 MHz): δ 8.17 (d, 1H, J=7.6 Hz), 7.54 (d, 1H, J=7.2 Hz), 7.43 (s, 1H), 7.39-7.30 (m, 3H), 7.26 (t, 1H, J=7.2 Hz), 7.22-7.12 (m, 2H), 5.37 (t, 1H, J=7.2 Hz), 4.48 (t, 1H, J=8.8 Hz), 3.05 (ddd, 1H, J=7.2, 7.6, 14.2 Hz), 2.22-2.13 (m, 1H), 1.67 (s, 9H).

### Preparation of Compound 6-5c.

A 10 mL extra dry THF solution of the compound 6-4c (95 mg, 0.27 mmol) was cooled below −15° C. 0.3 mL Et₃N was added drop-wise under argon. Then the reaction solution was cooled below −60° C., MsCl (104 mg, 0.54 mmol, 2 equiv.) in 250 uL extra dry THF was added drop-wise whilst the reaction temp was kept below −60° C. The reaction was rose to 0° C. and stirred for 2 hrs at the same temperature. A white suspension formed. Then 6 mL CH₃NH₂ saturated toluene was added. The reaction mixture was stirred at RT over weekend under argon.

TLC showed reaction complete. Solvent was removed under vacuum. The obtained residue was taken into 10 mL ethyl acetate and 10 mL H₂O, separated, the aqueous layer was extracted with ethyl acetate (5 mL×2). Organic was combined and dried over Na₂SO₄, filtered, concentrated and purified by flash chromatography to give the desired compound 6-5c (47 mg, ¹H-NMR confirmed the major product is trans isomer, 48% yield).

¹H-NMR (CDCl₃, 400 MHz): δ 8.16 (d, 1H, J=7.2 Hz), 7.46 (d, 1H, J=7.2 Hz), 7.38-7.16 (m, 7H), 4.81 (t, 1H, J=7.6 Hz), 4.33 (dd, 1H, J=4.0, 6.8 Hz), 2.57 (s, 3H), 2.56-2.43 (m, 2H), 1.68 (s, 9H).

### Preparation of Compound LNK-126 TFA Salt

To a 1 mL extra dry DCM solution of the compound 6-5c (30 mg, 0.08 mmol) was added 0.2 mL TFA (in 0.2 mL extra dry DCM) drop-wise below 0° C. After the addition, the reaction was stirred at room temp for 1 hr. TLC showed reaction complete. Solvent was removed under vacuum. 1 mL ether was added into the obtained oil, a white precipitate crashed out, collected to give the desired product as white solid (22 mg, 75% yield, the purity is less than 90%). The solid was washed with DCM, filtered to afford the compound LNK 126 trifluoroacetate as a white solid (8 mg, ¹H-NMR confirmed)

¹H-NMR (CD₃OD, 400 MHz): δ 8.18 (d, 1H, J=8.4 Hz), 7.70-7.60 (m, 1H), 7.46-7.42 (m, 3H), 7.32-7.03 (m, 4H), 5.00-4.88 (m, 2H), 2.80 (s, 3H), 2.78-2.70 (m, 2H). MS (ESI, positive)=263.1, HPLC 90.1%

### Example 6

### Preparation of Compound LNK-123 and LNK-124

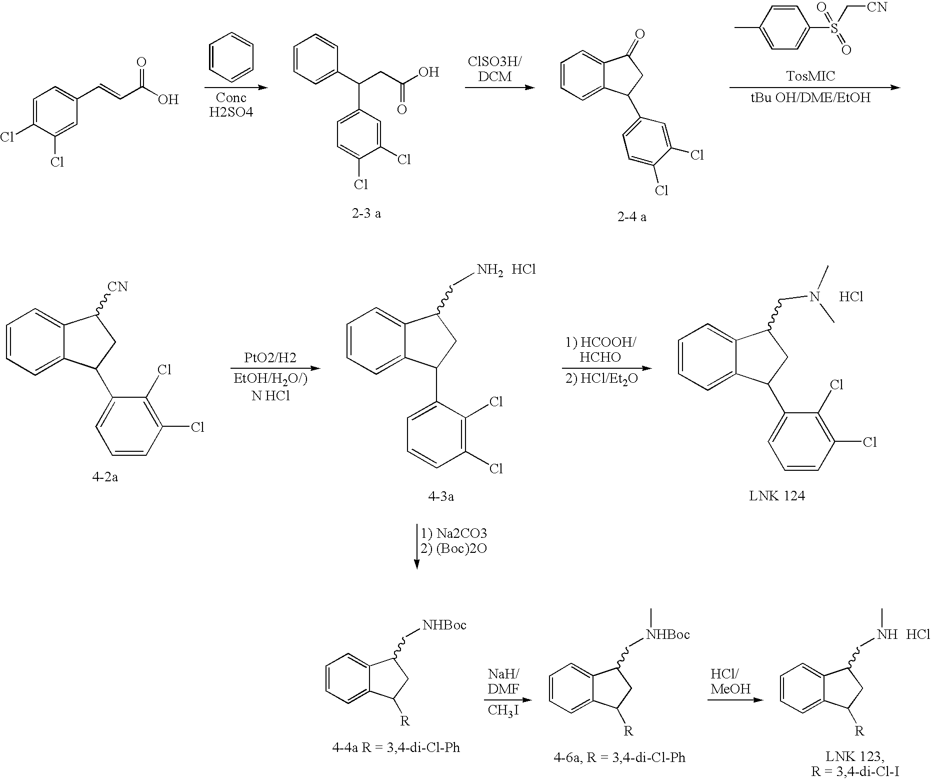

Preparation of Compound 2-3a.

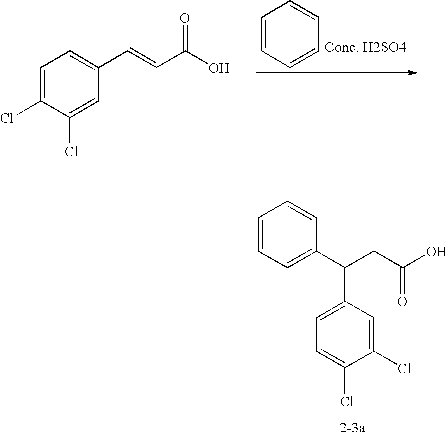

To the flask charged with 1.68 g of (E)-3-(3,4-dichlorophenyl)acrylic acid (7.7 mmol) was added benzene (6 mL) and concentrated H₂SO₄ (4.2 mL), and the mixture was heated to reflux overnight, TLC showed no starting material, the reaction mixture was poured into ice-water, stirred for 30 min, then separated, the water layer was extracted with EtOAc (3×25 mL). The organic layer combined was washed with water (5×20 mL), brine (2×25 mL) and dried, concentrated to give 2.146 g thick oil. This crude product was used for the next step without further purification.

Preparation of Compound 2-4a.

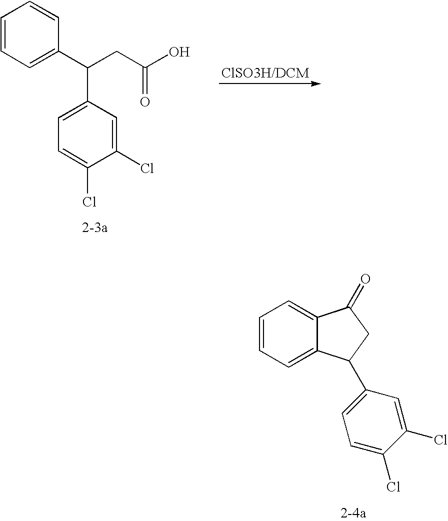

To the solution of compound 2-3a (2.146 g, 7.2 mmol) in 15 mL of DCM was added ClSO₃H (2 mL) via syringe, the solution turned to be dark immediately, then the mixture was stirred at RT overnight and poured into ice-water, then stirred for 30 min, then transferred to a separatory funnel. The cloudy bottom layer was collected and DCM was removed to give a yellow oil which dissolved in EtOAc. The aqueous layer was extracted with EtOAc (3×25 mL). The combined EtOAc layer was washed with water (5×25 mL), brine (2×25 mL), dried over anhydrous Na₂SO₄. After removing EtOAc, the residue was triturated with methanol, 883 mg of compound 2-4a was obtained as off-white solid (1H-NMR confirmed, 41% yield over two steps).

¹H-NMR (400 MHz, CDCl₃) δ: 2.64 (dd, J=19.2, 4.0 Hz, 1H), 3.25 (dd, J=19.2, 8.0 Hz, 1H), 4.57 (dd, J=8.0, 4.0 Hz, 1H), 6.97 (dd, J=8.4, 2.0 Hz, 1H), 7.25 (d, J=2.0 Hz, 1H), 7.28 (d, J=8.4 Hz, 1H), 7.41 (d, J=7.6 Hz, 1H), 7.48 (dd, J=7.6, 7.2 Hz, 1H), 7.63 (dd, J=7.6, 7.6 Hz, 1H), 7.85 (d, J=7.2 Hz, 1H).

### Preparation of Compound 4-2a.

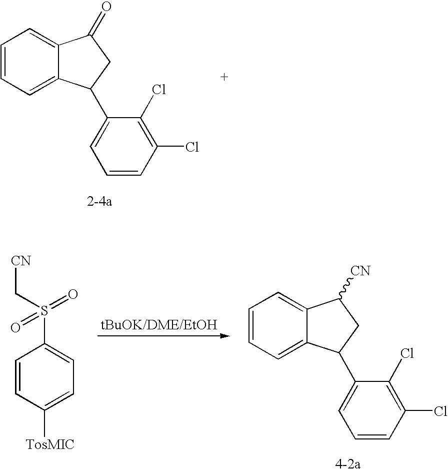

To an ice-cold solution of compound 2-4a (70 mg, 0.25 mmol) and TosMIC (146 mg, 0.75 mmol) in 1 mL of DME (dry) and 23 μL of absolute ethanol was added Kt-OBu (84 mg, 0.75 mmol) in three portions while keeping the reaction temperature between 5 and 10° C., after addition, TLC showed no ketone left, then the reaction mixture was stirred at RT overnight, TLC did not show lots of compound 4-2c formed, then another 0.2 mL of EtOH was added and heated to 40° C. for 6 hrs, the reaction mixture was purified by flash column to give 11 mg product 1 (¹H-NMR showed it is trans isomer of the desired product with some impurity, 15% yield) and 22 mg of product 2 (mixture of two isomers of compound 4-2a, 30% yield), NOESY indicated the first product is the trans isomer.

¹H-NMR (400 MHz, CDCl₃, trans-isomer) δ: 2.41-2.48 (m, 1H), 2.88-2.95 (m, 1H), 4.28 (dd, J=8.4, 4.2 Hz, 1H), 4.60 (t, J=7.8 Hz, 1H), 6.97 (dd, J=8.4, 2.0 Hz, 1H), 7.08 (d, J=7.2 Hz, 1H), 7.19 (d, J=2.0 Hz, 1H), 7.33-7.40 (m, 2H), 7.41 (dd, J=8.4 Hz, 1H), 7.52 (d, J=7.2 Hz, 1H).

¹H-NMR (400 MHz, CDCl₃, major cis-isomer) δ: 2.33 (dt, J=13.2, 10.4 Hz, 1H), 3.04 (dt, J=13.2, 7.6 Hz, 1H), 4.18 (dd, J=9.6, 8.0 Hz, 1H), 4.31 (m, 1H), 6.95 (d, J=6.8 Hz, 1H), 7.07 (dd, J=8.0, 2.0 Hz, 1H), 7.30-7.42 (m, 3H), 7.45 (d, J=8.4 Hz, 1H), 7.55 (d, J=8.0 Hz, 1H).

### Preparation of Compound 4-3a.

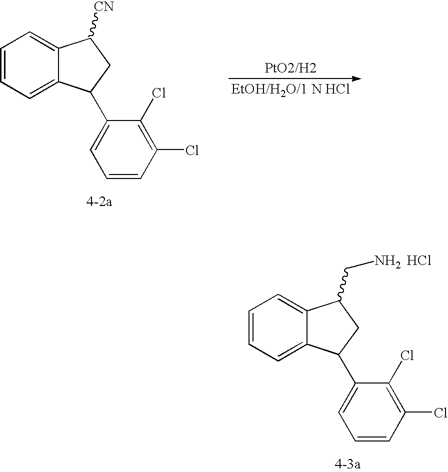

The mixture of crude compound 4-2a (crude weight 26 mg, 0.007 mmol, mixture of cis and trans isomers), 15 mg of PtO₂ in 2 mL of EtOH, 0.8 mL of H₂O and 0.2 mL of 1 N HCl was hydrogenated at 0.4 MPa at RT for 2 hrs, TLC showed no starting material, the catalyst was filtered off, and the filtrate was concentrated and dried in vacuo to give 26 mg of crude compound 4-2a, which was dissolved in EtOAc, and washed with saturated Na₂CO₃, the organic layer was concentrated to give 25 mg of the free amine as yellow oil, this free amine was used for the next step without further purification.

### Preparation of Compound 4-4a.

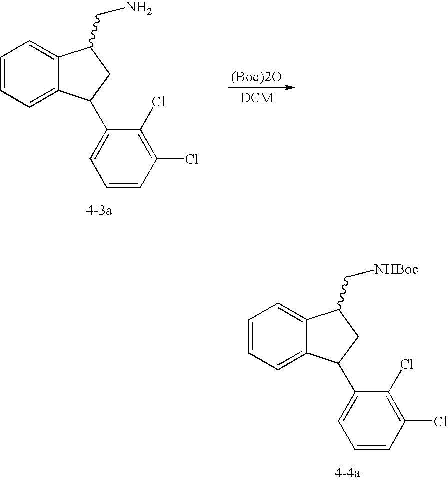

To the solution of compound 4-2a (25 mg, 0.085 mmol) in 2.5 mL of DCM was added (Boc)₂O (25 mg, 0.12 mmol) at RT. After 2 hrs, TLC showed reaction was complete, solvent was removed to give 51 mg of crude product (¹H-NMR confirmed), the crude product was purified by flash column eluting with n-hexane and n-hexane/EtOAc 5/1 to give 24 mg pure compound 4-4a.

¹H-NMR (400 MHz, CDCl₃, crude product, ratio of trans to cis isomer is about 0.85 to 1) (trans-isomer) δ: 1.44 (s, 9H), 2.15-2.23 (m, 1H), 2.38-2.45 (m, 1H), 3.31-3.43 (m, 2H), 3.75-3.85 (m, 1H), 4.41 (t, J=8.0 Hz, 1H), 4.63 (brs, 1H, NH), 6.97-6.99 (m, 2H), 7.20-7.35 (m, 4H), 7.36 (d, J=8.4 Hz, 1H).

Cis-isomer: δ:1.47 (s, 9H), 1.70-1.79 (m, 1H), 2.71-2.78 (m, 1H), 3.31-3.42 (m, 3H), 4.25 (t, J=8.4 Hz, 1H), 4.60-4.70 (m, 1H, NH), 6.92 (d, J=7.6 Hz, 1H), 7.05 (dd, J=8.4, 2.0 Hz, 1H), 7.20-7.34 (m, 4H), 7.40 (d, J=8.0 Hz, 1H).

### Preparation of Compound 4-5a.

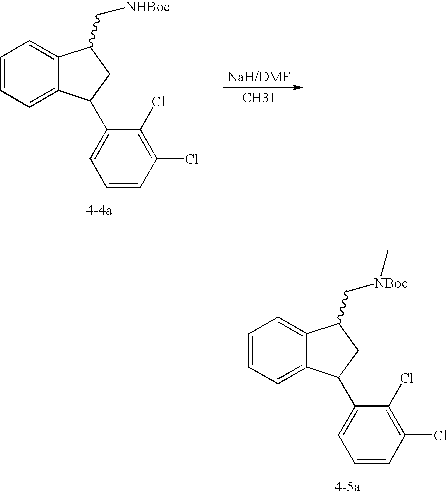

To the solution of compound 4-4a (24 mg, 0.061 mmol) in 1.5 mL of dry DMF was added NaH (12 mg, 0.5 mmol) in one portion at <10° C., the solution turned to be brown then yellow, then the reaction mixture was stirred at the same temperature for 15 min, then 0.08 mL of CH₃I was added, the reaction was allowed to RT then stirred overnight, TLC showed reaction complete. The reaction mixture was diluted with 20 mL EtOAc, washed with water (2×10 mL), then saturated NaCl (aq.), the aqueous layer was re-extracted with EtOAc (10 mL) and the organic layer was washed with saturated NaCl, the combined organic layer was dried with anhydrous Na₂SO₄ and concentrated to give 26 mg yellow oil (confirmed by ¹H-NMR and MS).

¹H-NMR (400 MHz, CDCl₃, crude product, mixture of trans and cis isomer) (trans-isomer) δ: 1.40 (s, 9H), 2.10-2.21 (m, 1H), 2.36-2.44 (m, 1H), 2.90-2.97 (m, 3H), 3.24-3.62 (m, 2H), 3.78-3.88 (m, 1H), 4.39-4.47 (m, 1H), 6.94 (d, J=7.6 Hz, 1H), 7.02 (dd, J=8.4, 2.0 Hz, 1H), 7.20-7.35 (m, 4H), 7.38 (d, J=8.0 Hz, 1H). Cis-isomer: δ:1.44 (s, 9H), 1.72-1.82 (m, 1H), 2.73-2.78 (m, 1H), 2.85-2.97 (m, 3H), 3.24-3.62 (m, 3H), 4.23-4.26 (m, 1H), 4.60-4.70 (m, 1H, NH), 6.92 (d, J=Hz, 1H), 7.06 (dd, J=8.4, 2.0 Hz, 1H), 7.20-7.34 (m, 4H), 7.40 (d, J=8.0 Hz, 1H).

### Preparation of Compound LNK 123 HCl Salt.

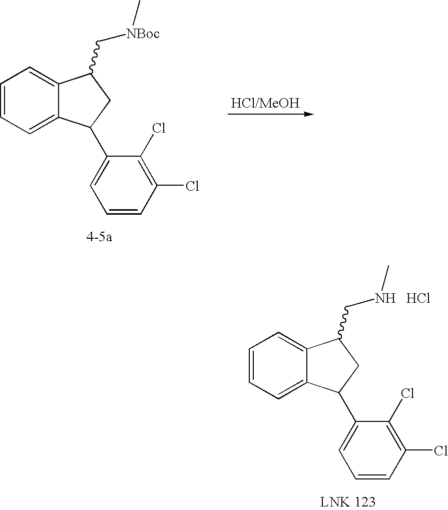

To a flask charged with compound 4-5a (26 mg, 0.064 mmol) was added 4 M HCl/MeOH (2 mL) via syringe at 0-5° C., then the reaction mixture was stirred at this temperature for 1 h, TLC showed lots of SM left, then the cooling bath was removed, after 3.5 h, still some 4-5a left by TLC, then the solvent was removed to give 29 mg of crude product as yellow solid, which was triturated in toluene, filtered to give 12 mg of desired product as off white solid (¹H-NMR and MS confirmed, 97% purity by HPLC, the ratio of trans-isomer to cis-isomer is about 0.7 to 1 by ¹H-NMR).

¹H-NMR (400 MHz, CD₃OD, mixture of trans and cis isomer) (trans-isomer) δ: 2.28-2.35 (m, 1H), 2.46-2.54 (m, 1H), 2.787 (s, 3H), 3.11-3.21 (m, 1H), 3.37 (dd, J=8.4, 4.4 Hz, 1H), 3.64-3.70 (m, 1H), 4.57 (dd, J=7.6, 7.6 Hz, 1H), 7.02 (d, J=6.8 Hz, 1H), 7.07 (dd, J=8.4, 2.0 Hz, 1H), 7.23-7.39 (m, 4H), 7.45 (d, J=8.4 Hz, 1H). Cis-isomer: δ:1.75-1.83 (m, 1H), 2.81 (s, 3H), 2.85-2.92 (m, 1H), 3.11-3.21 (m, 1H), 3.54-3.62 (m, 1H), 3.75 (dd, J=8.4, 4.0 Hz, 1H), 4.40 (dd, J=10.0, 8.0 Hz, 1H), 6.91 (d, J=7.6 Hz, 1H), 7.18 (dd, J=8.0, 2.0 Hz, 1H), 7.23-7.39 (m, 3H), 7.40 (d, J=2.0 Hz, 1H), 7.50 (d, J=8.0 Hz, 1H). HPLC: 97.5% MS (ESI+): 307.2 (M+1).

### Preparation of Compound LNK-124 HCl Salt

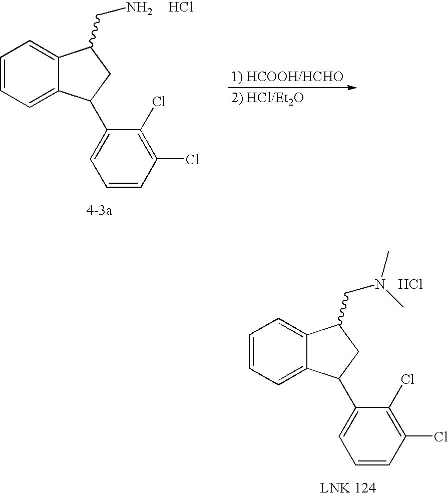

To a tube charged with 9 mg of compound 4-3a was added 0.5 mL of a mixed solution (25 mL HCOOH and 5 mL HCHO), the tube was sealed and heated to 90° C. (oil bath) and stirred overnight. TLC showed no starting material. The reaction was cooled to RT and basified with sat. NH₄OH, extracted with EtOAc, the organic layer was concentrated and HCl/Et₂O was added and concentrated to give 31 mg crude product as yellow solid, which was triturated with toluene to give 12 mg yellow solid, this yellow solid was washed with DCM, and DCM solution was dried to give 7 mg of desired product as pale yellow solid (¹H-NMR confirmed).

¹H-NMR (400 MHz, CDCl₃, mixture of trans and cis isomer) (trans-isomer) δ: 2.42 (dt, J=13.2, 7.6 Hz, 1H), 2.83-2.87 (m, 1H), 2.88-2.93 (m, 6H), 2.98-3.07 (m, 1H), 3.17-3.24 (m, 1H), 3.78-3.85 (m, 1H), 4.55-4.60 (m, 1H), 7.01-7.07 (m, 2H), 7.20 (d, J=2.0 Hz, 1H), 7.25-7.34 (m, 3H), 7.37 (d, J=8.4 Hz, 1H), 12.77 (brs, 1H). Cis-isomer: δ:1.92 (dt, J=12.8, 10.0 Hz, 1H), 2.93 (d, J=4.8 Hz, 3H), 2.96 (d, J=4.8 Hz, 3H), 3.12 (ddd, J=13.6, 10.0, 4.4 Hz, 1H), 3.26 (dt, J=13.6, 7.2 Hz, 1H), 3.68-3.74 (m, 1H), 3.79-3.86 (m, 1H), 4.30-4.35 (m, 1H), 6.96 (d, J=8.4 Hz, 1H), 7.06 (dd, J=8.4, 2.0 Hz, 1H), 7.25-7.34 (m, 4H), 7.42 (d, J=7.6 Hz, 1H), 12.93 (brs, 1H). HPLC: 93.8% MS (ESI⁺): 321.9 (M+2)

### Example 6

### Preparation of LNK-1110, 1111, 1114, 1115

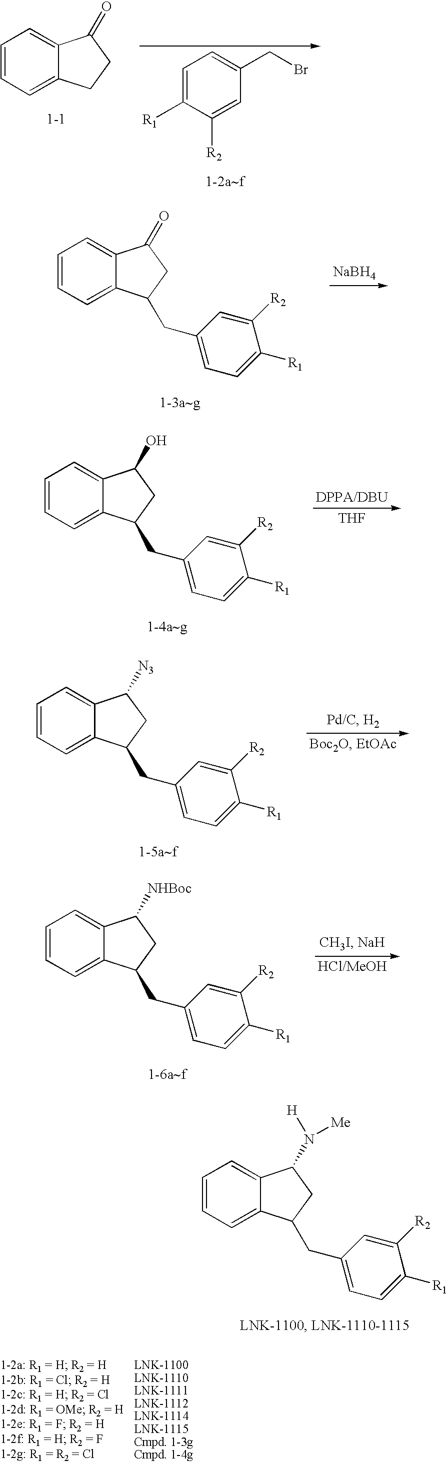

### Preparation of Compound LNK-1110

### Preparation of Compound 1-3b

LDA (2 M in THF/Heptane, 22.7 mL, 45.6 mmol, 3.0 eq) in 10 mL of dry THF was cooled to −75° C. under Ar, then compound 1-1 (2.0 g, 15.2 mmol, 1.0 eq) in THF (20 mL) was added into it in a period of 50 min. The reaction was stirred at −75° C. for 1 h, then for 2.5 hrs without cooling. Compound 1-2b (3.4 g, 16.6 mmol, 1.1 eq) in THF (10 mL) was added dropwise rapidly at about −10° C., and then the mixture was stirred at −10° C. for 1 h, then at RT overnight. The reaction was quenched with 50 mL of NaCl (saturated solution), 50 mL of HCl (3N) at −10˜−20° C., extracted with Et₂O (3×60 ml), the organic layer combined was washed with Na₂CO₃ (sat. 2×30 mL), brine (30 mL), dried over Na₂SO₄. The solvent was removed and the residue was purified by flash chromatography to give product as yellow solid (1.13 g). The impure fractions were concentrated and purified by flash chromatography again to give product as yellow solid (749 mg, 48% total yield).

### Preparation of Compound 1-4b

Compound 1-3b (819 mg, 3.20 mmol) was suspended in 20 mL of MeOH, and then cooled to 0° C. 140 mg of NaBH₄ was added into the reaction mixture at 0° C. The mixture was stirred at R.T overnight. The solvent of reaction was removed. The residue was treated with water (20 mL), extracted with ether (3×30 mL), washed with brine (30 mL), dried over Na₂SO₄, and concentrated to give crude product as slightly yellow solid (783 mg, yield 95%).

### Preparation of Compound 1-5b

DPPA (1.163 g, 4.23 mmol) was added to the solution of compound 1-4b (780 mg, 3.02 mmol) in 10 mL of dry THF. The mixture was stirred for 10 mins and cooled to 0° C. DBU (0.62 mL, 4.23 mmol) was added dropwise via syringe and the reaction mixture was allowed to warm to R.T overnight. The solvent of reaction was concentrated, and the residue was partitioned between water (30 mL) and ether (30 mL). The aqueous layer was extracted with ether (3×30 mL), and the combined extracts was washed with brine (20 mL), dried over Na₂SO₄, and concentrated to give crude product as yellow oil (1.050 g, yield>100%).

### Preparation of Compound 1-6b

10% Pd/C (75 mg) and (Boc)₂O (855 mg, 3.92 mmol) were added to the solution of compound 1-5b (860 mg, 3.02 mmol) in 15 mL of ethyl acetate. The mixture was stirred under H₂ at RT overnight. The reaction mixture was filtered and concentrated to give crude product as yellow oil. The crude product was purified by flash chromatography (PE:EA=50:1) to give the desired product as white solid (520 mg, white solid)

### Preparation of Compound 1-7b

To the solution of compound 1-6b (520 mg, 1.45 mmol) in 8 mL of dry DMF was added NaH (290 mg, 7.25 mmol) at <10° C. The reaction mixture was stirred at the same temperature for 15 min. Then 1.4 mL of CH₃I was added in. The reaction mixture was stirred at RT overnight. After TLC detection showing no raw material exists, the reaction mixture was diluted with 30 mL of EtOAc, washed with water (10 mL×2), and then saturated NaCl (10 mL×3). The organic layer was dried over anhydrous Na₂SO₄ and concentrated to give crude product as yellow oil (575 mg).

### Preparation of Compound LNK-1110

To a flask charged with compound 1-7b (575 mg, 1.55 mmol) was added MeOH (5 mL) and 4 M HCl/MeOH (5 mL) via syringe, and then the reaction mixture was stirred at R.T overnight. The solvent was removed to give 550 mg of product as yellow solid. The crude product was recrystallized with 95% ethanol and then filtered to give 50 mg of product as white solid (white solid, ¹H NMR showed it is the mixture of two diastereomers with 1/0.15 ratio of cis/trans).

Preparation of cis-LNK-1110

A solution of compound 1-3b (300 mg, 1.17 mmol) in 10 mL of MeOH and 5.17 ml of the solution of CH₃NH₂/MeOH (27-32%) was stirred at RT overnight. 69 mg of NaBH₄ was added into the reaction mixture. The mixture was stirred at RT for 4 hrs. The solvent was removed and the residue was taken into 20 mL of EtOAc, washed with water (2×15 mL), dried over Na₂SO₄, and concentrated to give crude product (356 mg, yellow oil). Then the crude product was dissolved in 5 mL of methanol and 5 mL of 4N HCl/MeOH solution. The resulting reaction mixture was stirred overnight. The solvent was removed to give 425 mg of crude product (yellow solid). The crude product was recrystallized with 95% ethanol to give the desired product as white solid (180 mg).

### Preparation of Compound LNK-1111

### Preparation of Compound 1-3c

LDA (2M in THF/Heptane, 17.2 mL, 34.5 mmol, 3.0 eq) was cooled to −75° C. under Ar, then compound 1-1 (1.515 g, 11.5 mmol, 1.0 eq) in THF (24 mL) was added into it in a period of 50 min. The reaction was stirred at −75° C. for 1 h, then for 2.5 hrs without cooling. Compound 1-2c (3.0 g, 34.5 mmol, 1.3 eq) in THF (6 mL) was added dropwise rapidly at about −10° C., and stirred at −10° C. for 1 h, then at RT overnight. The reaction was quenched with 45 ml of NaCl (saturated solution), 45 ml of HCl (3 N) at −10-20° C., extracted with Et₂O (3×45 ml), the organic layer combined was washed with Na₂CO₃ (Sat. 2×20 ml), brine (20 ml), dried over Na₂SO₄. The solvent was removed and the residue was purified by flash chromatography (PE/EA=60:1) to give 1.279 g of product as a pale yellow solid (43% yield).

### Preparation of Compound 1-4c

Compound 1-3c (800 mg, 3.12 mmol) was suspended in 20 mL of MeOH, and then cooled to 0° C. 130 mg of NaBH₄ was added into the reaction mixture at 0° C. The mixture was then stirred at RT for 2 hrs. The solvent was removed and the residue was treated with water (20 mL), extracted with ether (3×30 ml), the organic layer combined was washed with brine (30 mL), dried over Na₂SO₄, and concentrated to give crude product as slight yellow solid (868 mg).

Then the crude product was treated with 3.5 mL solution of hexane/EA=9:1 for 30 mins in ice-cold bath, then filtered to give the desired product as white solid. (498 mg, 62% yield).

### Preparation of Compound 1-5c

DPPA (739 mg, 2.68 mmol) was added to a solution of compound 1-4c (495 mg, 1.92 mmol) in 6.5 mL of dry THF. The mixture was stirred for 10 mins and then cooled to 0° C. DBU (0.39 mL, 2.68 mmol) was added dropwise via syringe and the reaction mixture was allowed to warm to rt overnight. TLC shows that the starting material of 1-4c was exist. The reaction was then heated by oil bath (40° C.) for 2 hrs and 1-4c still exists. The mixture was partitioned between water (20 mL) and ether (20 mL) and separated. The aqueous layer was extracted with ether (3×30 mL), and the combined extracts was washed with brine (20 mL), dried over Na₂SO₄, and concentrated to give crude product as yellow oil (697 mg, 10% yield)

### Preparation of Compound 1-6c

10% Pd/C (48 mg) and (Boc)₂O (544 mg, 2.50 mmol) were added to the solution of compound 1-5c (547 mg, 1.92 mmol) in 12 mL of EtOAc. The mixture was stirred under H₂ at RT overnight. The reaction mixture was filtered and concentrated to give crude product as yellow oil which was purified by flash column (PE/EA=50:1) to afford the desired product as colorless foam (408 mg, 59% yield over two steps).

### Preparation of Compound 1-7c

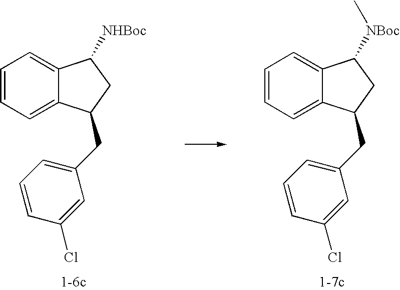

To the solution of compound 1-6c (394 mg, 1.10 mmol) in 5 mL of dry DMF was added NaH (132 mg, 5.49 mmol) at <10° C. The reaction mixture was stirred at the same temperature for 15 min. Then 0.95 mL of CH₃I was added in. The reaction mixture was stirred at RT overnight. After the reaction was completed by TLC, the reaction mixture was diluted with 20 mL of EA, washed with water (10 ml×2), and then saturated NaCl (10 ml). The organic layer was dried over anhydrous Na₂SO₄ and concentrated to give crude product as yellow oil (560 mg).

### Preparation of Compound LNK-1111 HCl Salt

To a flask charged with compound 1-7c (560 mg, 1.51 mmol) was added 4 M HCl/MeOH (5 mL) via syringe at 0-5° C., and then the reaction mixture was stirred at RT overnight. The solvent was removed to give 560 mg of crude product as yellow oil (560 mg). To the product was added 20 mL of ethyl acetate, then stirred overnight, and then filtered to give the title product as slightly yellow solid (360 mg), which was recrystallized with ethanol to give 89 mg of white solid.

### Preparation of Compound LNK-1114

### Preparation of Compound 1-3e

LDA (2M in THF, 34 mL, 68.1 mmol, 3 eq) in 40 mL of THF was cooled to −75° C. under Ar₂, then compound 1-1 (3 g, 22.7 mmol, 1.0 eq) in THF (40 mL) was added into it in a period of 40 min. The reaction was stirred at −75° C. for 1 h, then for 2.5 hrs without cooling. Compound 1-2e (5 g, 25.7 mmol, 1.13 eq) was added dropwise at about −10° C., stirred at −10° C. for 1 h and stirred at RT overnight. Then the reaction was quenched with 80 mL of NaCl (saturated solution), 80 mL of HCl (3N) at −10˜−20° C., extracted with Et₂O (3×50 ml), the organic layer combined was washed with Na₂CO₃ (sat, 50 mL), brine (50 mL), dried over Na₂SO₄. The solvent was removed and the residue was purified by column chromatography (PE/EA=25:1) to give 1.81 g of crude compound 1-3e as a yellow oil and 0.745 g pure compound 1-3e as a yellow oil. Total yield was 46%.

### Preparation of Compound 1-4e

Compound 1-3e (1.8 g, 7.5 mmol) was dissolved in 50 mL of MeOH, and then cooled to 0° C. 340 mg of NaBH₄ (9.0 mmol) was added into the reaction mixture at 0° C. The mixture was stirred at R.T over night. The solvent of reaction was removed. The residue was treated with water (30 ml), extracted with ether (3×50 ml), washed with brine (30 ml). The solvent was removed. The residue was treated with water (30 mL), extracted with ether (3×50 mL), washed with brine (30 mL), dried over Na₂SO₄. The solvent was concentrated to give crude compound 1-4e as a white solid. The crude product was recrystallized with EA and Hexane to give 0.887 g of pure compound 1-4e as a white solid (yield: 49%) and 0.7 g of crude compound 1-4e as a white solid (yield: 38%).

### Preparation of Compound 1-5e

To the solution of compound 1-4e (880 mg, 3.62 mmol, 1.0 eq) in THF (20 mL) was added DPPA (1.4 g, 5.07 mmol, 1.4 eq), the mixture was stirred for 10 min and then cooled to 0° C., DBU (0.75 ml, 5.07 mmol, 1.4 eq) was added into the reaction via syringe and the mixture was warmed to r.t and stirred overnight. The mixture was partitioned between water (20 mL) and ether (20 mL), separated and the aqueous phase was extracted with ether (20 mL×3). The combined extracts were washed with brine (20 mL), dried over Na₂SO₄, concentrated and purified by flash chromatography (EA/PE=1:100) to give 0.833 g of crude compound 1-5e as a oil (yield: 86.2%).

### Preparation of Compound 1-6e

10% Pd/C (78 mg) and (Boc)₂O (830 mg, 3.74 mmol) were added to the solution of compound 1-5e (833 mg, 3.12 mmol) in 16 mL of EtOAc. The mixture was stirred under H₂ at RT overnight. The reaction mixture was filtered and concentrated to give crude product which was purified by flash column (PE/EA=10:1) to purified by flash chromatography (EA/PE=1:10) to give 0.95 g of compound 1-6e as a pale yellow oil (yield: 89.2%).

Preparation of cis-LNK-1114

To a solution of compound 1-3e (740 mg, 3.06 mmol) in 50 mL of MeOH was added 7.4 mL of the solution of CH₃NH₂/MeOH (27-32%), the resulted solution was stirred at rt for 3 hrs. 150 mg of NaBH₄ (4.0 mmol) was added into the reaction mixture. The mixture was stirred at RT overnight. The solvent was removed. The residue was taken into 20 mL of EtOAc, washed with water (2×15 ml), dried over Na₂SO₄, and concentrated. The residue was dissolved in 5 mL of methanol, and 5 mL of 4N HCl/MeOH solution was added. The result reaction mixture was stirred overnight. The reaction was concentrated, and partitioned between EA and water, the separated organic layer was washed with brine, dried over Na₂SO₄, concentrated and purified by flash chromatography (EA/MeOH=3:1) to give 0.508 g white solid. The white solid was recrystallized with EtOH (7.5 mL) to give 0.337 g of pure compound as a white solid (yield: 38%).

### Preparation of Compound LNK-1115

### Preparation of Compound 1-3f

LDA (2M in THF/Heptane, 22.7 ml, 45.6 mmol, 3.0 eq) was cooled to −75° C. under Ar, then compound 1-1 (2 g, 15.2 mmol, 1.0 eq) in THF (20 ml) was added into it in a period of 50 min. The reaction was stirred at −75° C. for 1 h, then for 2.5 hrs without cooling. Compound 1-2f (2.2 ml, 18.2 mmol, 1.2 eq) in THF (10 ml) was added dropwise rapidly at about −10° C., and stirred at −10° C. for 1 h. The reaction mixture was stirred over night. TLC shows that raw material still remains and the reaction mixture was heated to 30° C. for 4 hrs. The reaction was quenched with 50 ml of NaCl (Saturated solution), 50 ml of HCl (3N) at −10˜−20° C., extracted with Et₂O (3×60 ml), the organic layer combined was washed with NaHCO₃ (sat. 3×20 ml), brine (30 ml), dried over Na₂SO₄. The solvent was removed and the residue was purified by flash chromatography (PE/EA=80:1) to give 1.855 g of product as yellow oil (1.855 g, yellow oil, yield 51%).

### Preparation of Compound 1-4f

Compound 1-3f (1.28 g, 5.33 mmol) was dissolved in 15 ml of MeOH, and then cooled to 0° C. 232 mg of NaBH₄ (6.13 mmol) was added into the reaction mixture at 0° C. The mixture was stirred at RT overnight. The solvent of reaction was removed. The residue was treated with water (30 ml), extracted with ether (3×50 ml), washed with brine (30 ml). The solvent was removed. The residue was treated with water (30 mL), extracted with ether (3×50 mL), washed with brine (30 mL), dried over Na₂SO₄. The solvent was concentrated to give crude product as slight yellow solid (1.235 g, Cis:trans=5:1, yield 95.7%).

Then the crude product was treated with 7 ml solution of hexane/EA=9:1 and stirred for 20 mins, then filtered to give product as white solid. (780 mg).

### Preparation of Compound 1-5f

DPPA (1.24 g, 4.51 mmol) was added to a solution of 1-4f (780 mg, 3.22 mmol) in 10 ml of dry THF. The mixture was stirred for 10 mins and cooled to 0° C. DBU (0.66 ml, 4.51 mmol) was added dropwise via syringe and the reaction mixture was allowed to warm to RT overnight. The mixture was partitioned between water (20 ml) and ether (20 ml). The aqueous layer was extracted with ether (3×30 ml), and the combined extracts was washed with brine (20 ml), dried over Na₂SO₄, and concentrated to give crude product as yellow oil (1.110 g, yield>100%).

### Preparation of Compound 1-6f

10% Pd/C (102 mg) and (Boc)₂O (913 mg, 4.18 mmol) were added to the solution of compound 1-5c (860 mg, 3.22 mmol) in 10 ml of ethyl acetate. The mixture was stirred under H₂ at RT overnight. The reaction mixture was filtered and concentrated to give crude product. The crude product was purified by flash chromatography (PE:EA=40:1) to afford product as a colorless foam (690 mg, yield 63% over two steps).

### Preparation of Compound 1-7f

To the solution of compound 1-6f (690 mg, 2.02 mmol) in 6 ml of dry DMF was added NaH (242 mg, 10.12 mmol) in one portion at <10° C. The reaction mixture was stirred at the same temperature for 15 mins. Then 1.32 ml of CH₃I was added in. The reaction mixture was stirred at RT over night. After TLC detection showing no raw material exists, the reaction mixture was diluted with 25 mL of ethyl acetate, washed with water (10 ml×2), the aqueous layer was re-extracted with ethyl acetate (20 ml×2), and the combined ethyl acetate layers were washed with saturated NaCl (10 ml×2). The organic layer was dried over anhydrous Na₂SO₄ and concentrated to give crude product as yellow oil (730 mg).

### Preparation of LNK-1115

To a flask charged with compound 1-7f (730 mg, 2.06 mmol) was added 4 M HCl/MeOH (5 mL) via syringe, and then the reaction mixture was stirred at RT overnight. The solvent was removed to give crude product as yellow solid (750 mg). Product was recrystallized by 95% ethanol and filtered to give the product as white solid (429 mg).

Preparation of cis-LNK-1115

A solution of compound 1-3f (425 mg, 1.77 mmol) in 15 ml of MeOH and 7.8 ml of the CH₃NH₂/MeOH (27-32%) was stirred at RT overnight. 104 mg of NaBH₄ (2.76 mmol) was added into the reaction mixture. The mixture was stirred at RT for 5 hrs. The solvent of reaction was removed. The residue was taken into 30 mL of ethyl acetate, washed with water (2×15 ml), dried over Na₂SO₄, and concentrated to give crude product 460 mg as yellow oil. Then the crude product was dissolved in 5 ml of methanol and 5 ml of 4N HCl/MeOH solution. The result reaction mixture was stirred overnight. The solvent was removed and gave 500 mg of crude product as yellow solid. The crude product was recrystallized by 95% ethanol, filtered to give the desired product as white solid (188 mg).

### Example 8

### Preparation for LNK-1521

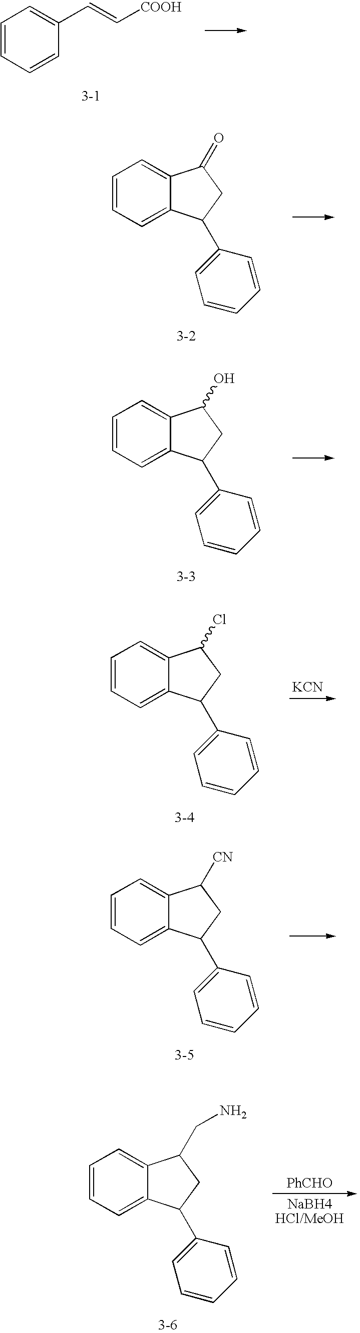

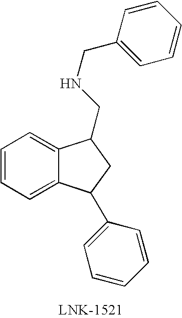

### Preparation of Compound 3-2

To a solution of 30 g of compound 3-1 (202.48 mmol) in benzene (150 mL) was added AlCl₃ (108 g, 809.96 mmol) in a slowly rate. The mixture was heated to reflux overnight. TLC showed no SM. The mixture was poured into ice-water and stirred, then extracted with ethyl ether. Then ether layer combined was washed with water, 10% NaOH and brine, dried and concentrated to give red solid which was then triturated with PE and isopropyl ether to give 26 g compound 3-2 as off-white solid (yield 61.7%).

### Preparation of Compound 3-3

To the flask charged with 10 g of compound 3-2 was added THF (50 mL) and stirred in ice bath. Then a solution of NaBH₄/H₂O (3.6 g/20 mL) which was pre-cooled in ice-bath was added dropwise to the flask, while the reaction temperature was maintained at −5˜0° C. After all of NaBH₄ was added, the cooling bath was removed. And the mixture was stirred at RT overnight. TLC (PE:EA=5:1) showed no S.M. The mixture was diluted with water and stirred for 30 min. Then THF was removed under reduced pressure and aqueous phase was extracted with ethyl acetate. The organic phase was washed with water, brine then dried over Na₂SO₄, concentrated to give 9.3 g of off-white solid (yield 92.1%).

### Preparation of Compound 3-4

To the solution of 9.3 g of comp 3-3 (44.23 mmol) in dry 80 mL of dioxane cooled with an ice-bath was added 6.5 mL of SOCl₂ in 15 min, then the mixture was stirred overnight. TLC showed reaction complete (PE:EA=5:1). The mixture was concentrated and dissolved in EA. Then EA phase was washed with water, brine then dried over Na₂SO₄, and concentrated to give 10.1 g of crude compound 3-4 as light yellow solid (yield 100%).

### Preparation of Compound 3-5

To the solution of 10.1 g of comp 3-4 (44.16 mmol) in dry 40 mL of DMF was added NaCN (2.6 g, 52.99 mmol), then the mixture was stirred at 50˜60° C. (oil bath) overnight. TLC showed reaction completed. The mixture was diluted with water and then extracted with ethyl acetate. Then EA phase was washed with water, brine then dried over Na₂SO₄, Then concentrated to give dark oil which was then purified by silica column to give 6.68 g of comp 3-5 as yellow oil (yield 69.0%).

### Preparation of Compound 3-6

To a solution of compound 3-5 (200 mg, 0.912 mmol) in 4.5 mL of EtOH, 2 mL of H₂O and 1.5 mL of 1N HCl was added PtO₂ (124 mg). The mixture was hydrogenated under 0.2 MPa H₂ atmospheres at RT overnight. TLC showed no S.M. The mixture was diluted with aqueous Na₂CO₃ and the catalyst was removed. Then the filtrate was extracted with ethyl acetate. The organic layer combined was washed with water, brine, dried, and concentrated to give 150 mg of desired product (yield 73.6%).

### Preparation of LNK-1521

To the solution of 200 mg of comp 3-6 (0.896 mmol) in 5 mL of MeOH was added benzaldehyde (0.1 mL). Then the mixture was stirred for 1 h. TLC showed no compound 3-6. Then 67.8 mg of NaBH₄ (1.792 mmol) was added and the mixture was stirred at RT overnight. The mixture was diluted with water. Then methanol was removed under reduced pressure. The aqueous phase was extracted with ethyl acetate. The organic phase was washed with water, brine, dried, and concentrated to give 256 mg of crude product as colorless oil (91.4% yield). This crude product was converted to HCl salt with HCl/MeOH (300 mg of crude product) which was recrystallized with 95% EtOH to give 155 mg of white solid (¹H NMR confirmed to be the desired product but with some impurity). The mother liquid was concentrated and washed with ethyl acetate to give 55 mg of white solid (¹H NMR showed the majority was trans-isomer). Product was again recrystallized to give 83 mg of white solid (confirmed by ¹H NMR to be cis-LNK-1521). And the mother liquid was concentrated and washed by ether to give 50 mg of white solid (¹H NMR showed the ratio of trans/cis was 2:1).

Product was then washed by hexane/EA (9:1) to give 50 mg solid which was again recrystallized with EtOH to give 41 mg of white solid (¹H NMR showed it was pure trans isomer).

### Example 9

### Preparation of LNK-1800

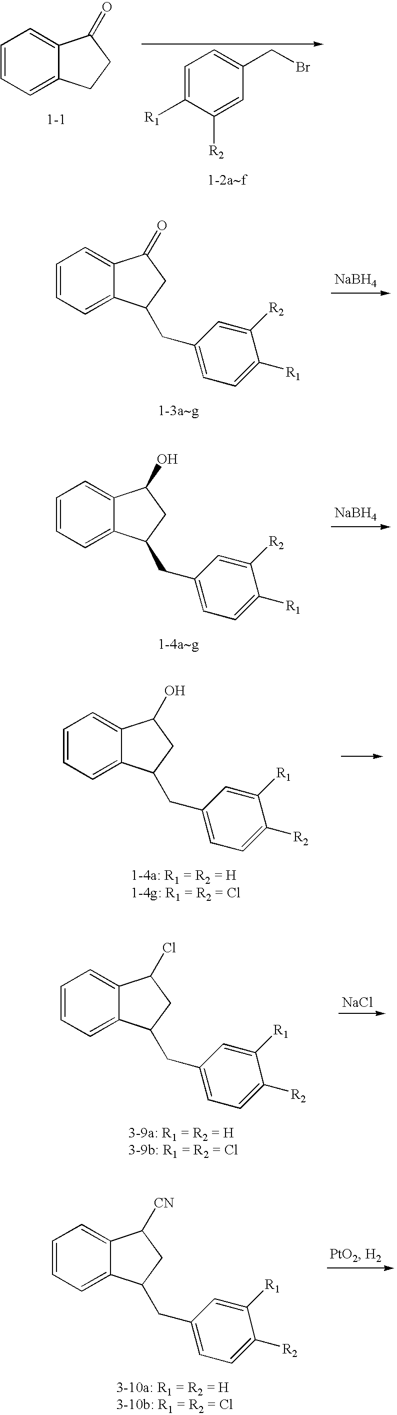

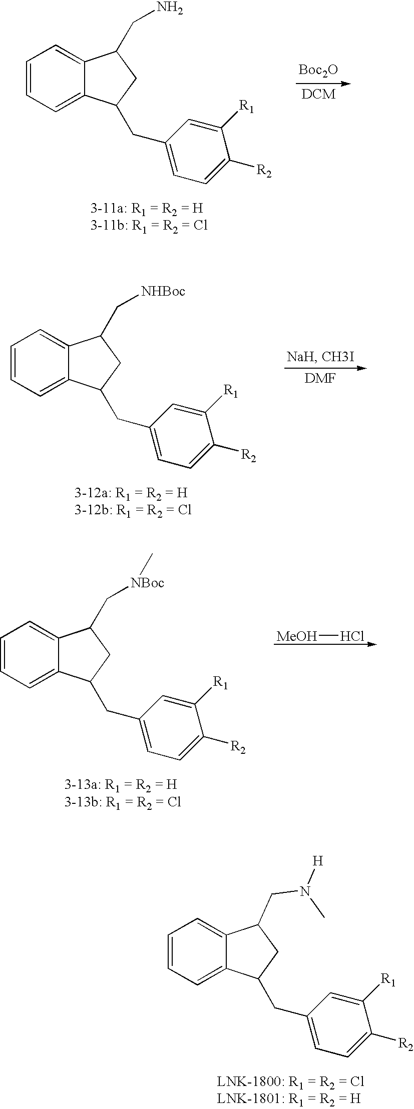

### Preparation of 1-3g

LDA (2 M in THF/Hept., 90 mL, 0.18 mol, 3.0 eq) and THF (90 mL) were cooled to −75° C. under N₂, then compound 1-1 (8 g, 0.06 mol, 1.0 eq) in THF (40 mL) was added into it in a period of 1 h, the reaction was stirred at −75° C. for 1 h, then for 2.5 hrs without cooling. Compound 1-2g (18.9 g, 0.078 mol, 1.3 eq) in THF (10 mL) was added dropwise rapidly at about −10° C. and stirred at −10° C. for 1 h, then overnight without cooling. TLC showed the starting material almost disappeared, then the reaction was quenched with 100 mL of NaCl (saturated solution) and 100 mL of HCl (3 N) at <−10° C., extracted with Et₂O, the organic layer combined was washed with Na₂CO₃ (Sat., twice), brine (twice), dried over Na₂SO₄. The solvent was removed to give 29.8 g crude product. After purified by flash column for several times 5.671 g of pure compound 1-3g was obtained (32% yield).

### Preparation of Compound 1-4g

Compound 1-3g (5.99 g, 20.57 mmol, 1.0 eq) was suspended in MeOH (80 mL), then cooled to 0° C., then NaBH₄ (0.855 g, 23 mmol) was added at 0° C., the reaction was stirred at RT for 2 hrs, TLC showed no starting material. The solvent was removed and the residue was treated with H₂O, extracted with Et₂O, the organic layer combined was washed with brine, dried over Na₂SO₄, and concentrated to give 5.71 g of crude compound 1-4g as yellow solid (crude yield 95%).

### Preparation of Compound 3-9b

To the solution of compound 1-4g (2.891 g, 9.8 mmol, 1.0 eq) in dry dioxane (50 mL) was added 1.8 mL of SOCl₂ while cooling with ice-bath, then the reaction was stirred at RT overnight. The solvent was removed, then the residue was treated with H₂O, extracted with ethyl acetate, washed with brine, the organic layers were combined and dried over Na₂SO₄, concentrated to give the desired product (3.137 g, 103%).

### Preparation of Compound 3-10b

To the solution of compound 3-9b (5.39 g, 17.3 mmol, 1.0 eq) in 80 ml of DMF was added 1.272 g NaCN (25.95 mmol), then the reaction was stirred at 70° C. (oil bath) overnight. The reaction mixture was diluted with water, extracted with ethyl acetate, then combined the organic layers was washed with brine, dried over Na₂SO₄, concentrate to give red black oil which was further purified by flash chromatography (P→P:E=50:1) to give 615 mg of byproduct as yellow oil, 1.401 g of major cis-isomer as yellow oil, 1.35 g of mix-isomer and 644 mg of major trans-isomer.

### Preparation of Mix-Compound 3-11b

The mixture of Compound 3-10b (427 mg, 1.413 mmol, ¹H NMR showed it is a mixture of cis and trans-isomer), 210 mg of PtO₂ in 7 mL of EtOH, 3 mL of water, and 2.5 mL of 1 N HCl was hydrogenated at 0.5 MPa at RT overnight. The catalyst was filtered off, and the filtrate was concentrated and the residue was dissolved in EA, washed with saturated Na₂CO₃, the organic layer was concentrated to give 428 mg of crude product as black oil, the crude product was used for the next step without further purification, crude yield 99%.

### Preparation of Cis-Compound 3-11b

The mixture of cis-compound 3-10b (250 mg, 0.827 mmol, cis-isomer), 123 mg of PtO₂ in 4 mL of EtOH, 1.8 mL of water and 1.5 mL of 1 N HCl was hydrogenated at 0.5 MPa at RT overnight. The catalyst was filtered off and the filtrate was concentrated, then was dissolved in ethyl acetate, washed with saturated Na₂CO₃, the organic layer was concentrated to give 238 mg of black oil, this crude product was used for the next step without further purification, crude yield 94%).

### Preparation of Trans-Compound 3-11b

The mixture of compound 3-10b (644 mg, 2.131 mmol, trans-isomer), 522 mg of PtO₂ in 10 mL of EtOH, 4.5 mL of water and 4 mL of 1 N HCl was hydrogenated at 0.5 MPa at RT overnight. The catalyst was filtered off and the filtrate was concentrated. The crude product was dissolved in ethyl acetate, washed with saturated Na₂CO₃, the organic layer was concentrated to give 493 mg of free amine (crude yield 75%).

### Preparation of Mix-Compound 3-12b

To the solution of mix-compound 3-11b (426 mg, 1.391 mmol) in 12 mL of DCM was added (Boc)₂O (580 mg, 2.782 mmol) at RT for 2 hrs. The solvent was removed to give 780 mg of crude product as black oil.

### Preparation of Cis-Compound 3-12b

To the solution of cis-compound 3-11b (1.12 g, 3.661 mmol) in 30 mL of DCM was added (BoC)₂O (0.915 g, 4.393 mmol) at RT for 2 hrs. The solvent was removed to give 1.88 g of yellow oil, it was further purified by flash chromatography (P:E=50:1-25:1-20:1) to give 1.210 g of the desired product as white solid (yield 81%).

### Preparation of Trans-Compound 3-12b

To the solution of trans-compound 3-11b (493 mg, 1.61 mmol) in 10 mL of DCM was added (BoC)₂O (402 mg, 1.93 mmol) at RT. After 2 hrs, the solvent was removed to give 850 mg yellow oil, it was further purified by flash chromatography (P:E=50:1-25:1-20:1) to give 502 mg of the desired product as pale yellow solid (yield 77%).

### Preparation of Mix-Compound 3-13b

To the solution of crude mix-compound 3-12b (780 mg, ˜1.35 mmol) in 20 mL of DMF (dry) was added NaH (130 mg, 5.41 mmol) in one portion at <10° C., the reaction mixture was stirred at the same temperature for 15 min, then 2 mL of CH₃I was added, the reaction was allowed to r.t and stirred overnight. The reaction mixture was diluted with ethyl acetate, washed with water, then saturated NaCl (aq.), the aqueous layer was re-extracted with ethyl acetate, and the organic layer was washed with saturated NaCl, the combined organic layer was dried with anhydrous Na₂SO₄, and concentrated to give 696 mg yellow oil. After further purification by flash chromatography, 90 mg of desired product was obtained.

### Preparation of Cis-Compound 3-13b

To the solution of crude cis-compound 3-12b (300 mg, 0.738 mmol) in 10 mL of DMF (dry) was added NaH (71 mg, 2.952 mmol, 4 eq) in one portion at <10° C., the reaction mixture was stirred at the same temperature for 15 min, then 1 mL of CH₃I was added, the reaction was allowed to RT then stirred overnight. The reaction mixture was diluted with EtOAc, washed with water, then saturated NaCl (aq.), the aqueous layer was re-extracted with ethyl acetate, and the organic layer was washed with saturated NaCl, the combined organic layer was dried with anhydrous Na₂SO₄, and concentrated to give 293 mg of the crude product as yellow oil. The crude product was further purified by flash chromatography to give 78 mg of pale yellow oil.

### Preparation of Trans-Compound 3-13b

To the solution of trans-compound 3-12b (477 mg, 1.174 mmol) in 15 mL of DMF (dry) was added NaH (113 mg, 4.695 mmol) in one portion at <10° C., the reaction mixture was stirred at the same temperature for 15 min, then 2 mL of CH₃I was added, the reaction was allowed to r.t then stirred overnight. The reaction mixture was diluted with ethyl acetate, washed with water, then saturated NaCl (aq.), the aqueous layer was re-extracted with ethyl acetate and the organic layer was washed with saturated NaCl, the combined organic layer was dried with anhydrous Na₂SO₄, and concentrated to give 525 mg of the desired product as yellow oil.

### Preparation of Mix-LNK-1800

To a flask charged with mix-compound 3-13b (90 mg, 0.214 mmol) was added 4 M HCl/MeOH (10 mL) via syringe at 0-5° C., and then the reaction mixture was stirred at RT for 2 hrs. The solvent was removed and the residue was partitioned between saturated NaHCO₃ and EtOAc, separated and the organic layer was dried and concentrated to give 79 mg of yellow oil (¹H NMR confirmed to be the desired product). Then this yellow oil was added 4 M HCl/MeOH (10 mL) via syringe at 0-5° C., and then the reaction mixture was stirred at RT for 2 hrs. The solvent was removed to give 197 mg of yellow solid, which was washed with CHCl₃ to give 44 mg as white solid, and the filtrate was concentrated to give 117 mg yellow solid.

### Preparation of Cis-LNK-1800

To a flask charged with cis-compound 3-13b (1.24 g, 2.95 mmol) was added 4 M HCl/MeOH (25 mL) via syringe at 0-5° C., and then the reaction mixture was stirred at RT for 2 hrs. The solvent was removed to give 1.336 g of yellow solid, the yellow solid was further recrystallized by EtOH to give 0.974 g of white solid (¹H NMR, MS, HPLC (95%) confirmed it be the desired compound, yield 93%).

### Preparation of Trans-LNK-1800

To a flask charged with trans-compound 3-13b (185 mg, 0.44 mmol) was added 4 M HCl/MeOH (5 mL) via syringe at 0-5° C., and then the reaction mixture was stirred at R.T for 2 hrs. The solvent was removed to give yellow solid, the yellow solid was further recrystallized by EtOH to give 185 mg of white solid (HPLC 89% purity). This product was further recrystallized by EtOH to give 135 mg of white solid (HPLC 92%), confirmed by ¹H NMR, MS to be the title compound), which was further recrystallized by EtOH to give 93 mg of desired product as white solid (HPLC 98%).

### Example 10

### Indatraline and Other Inventive Compounds Bind to α-Synuclein and Affect the Rate of Structure Formation in the Presence of HFIP

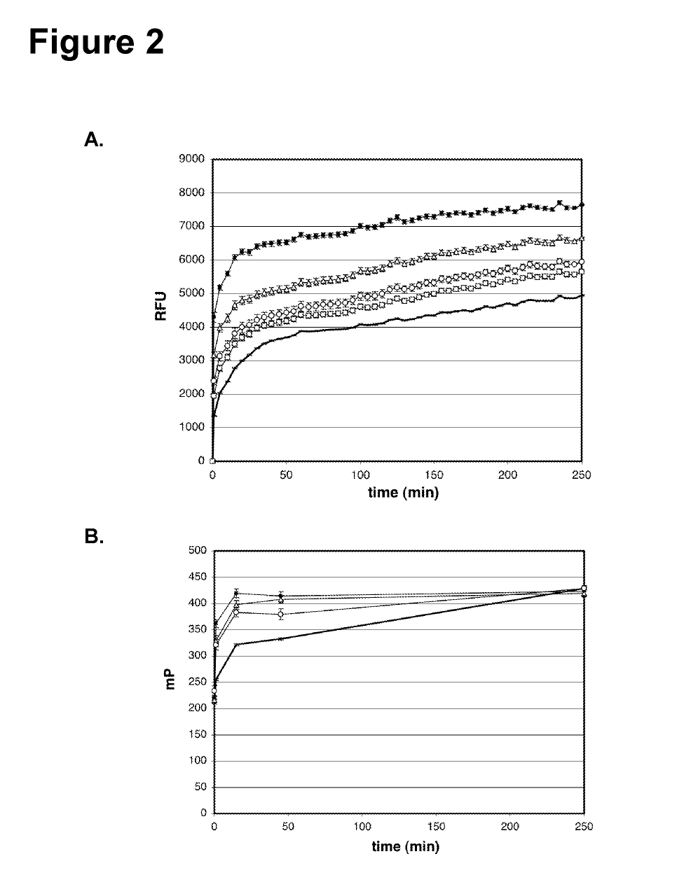

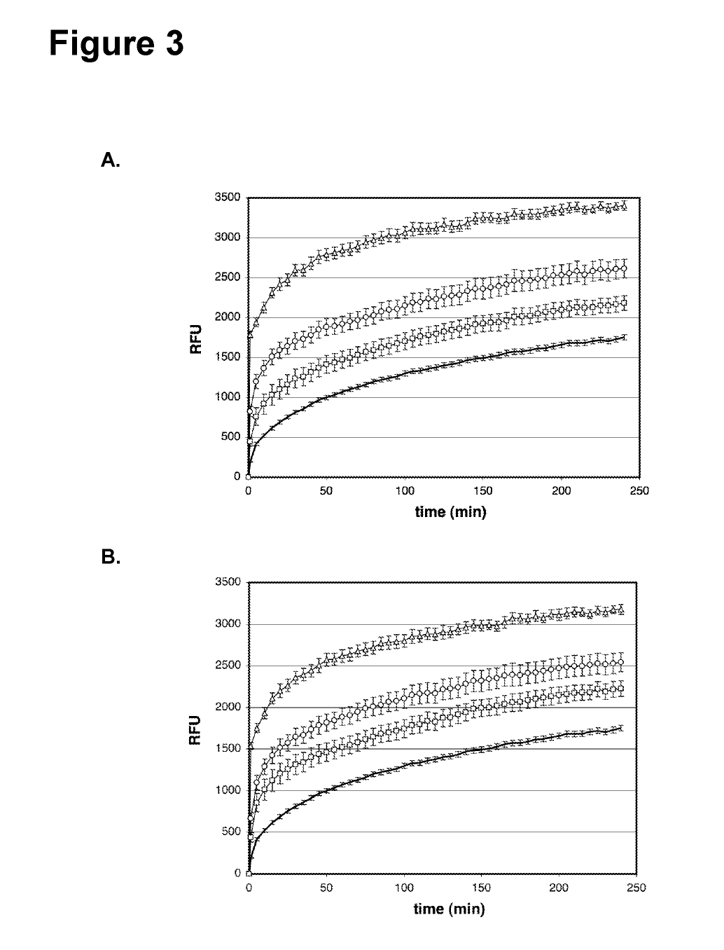

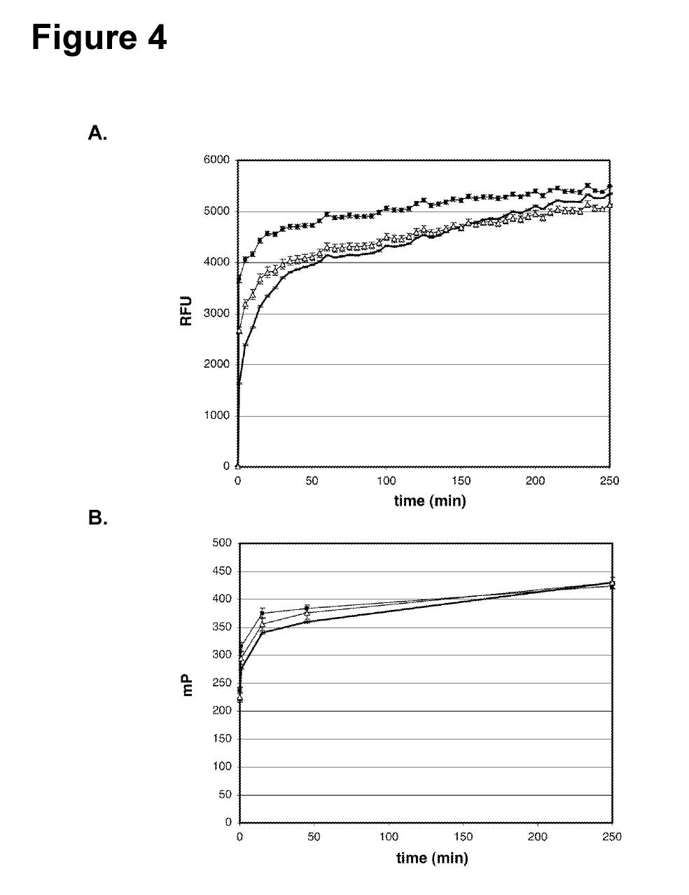

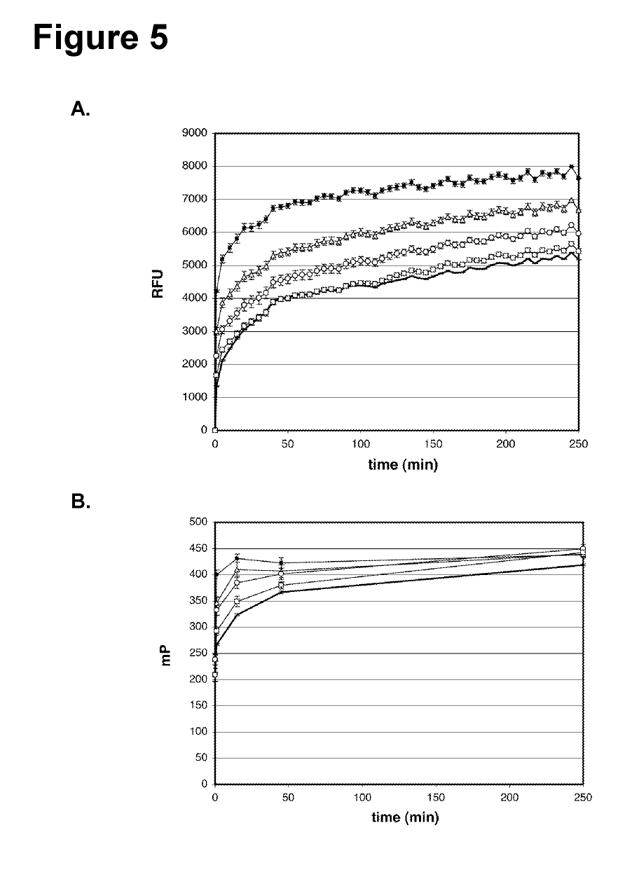

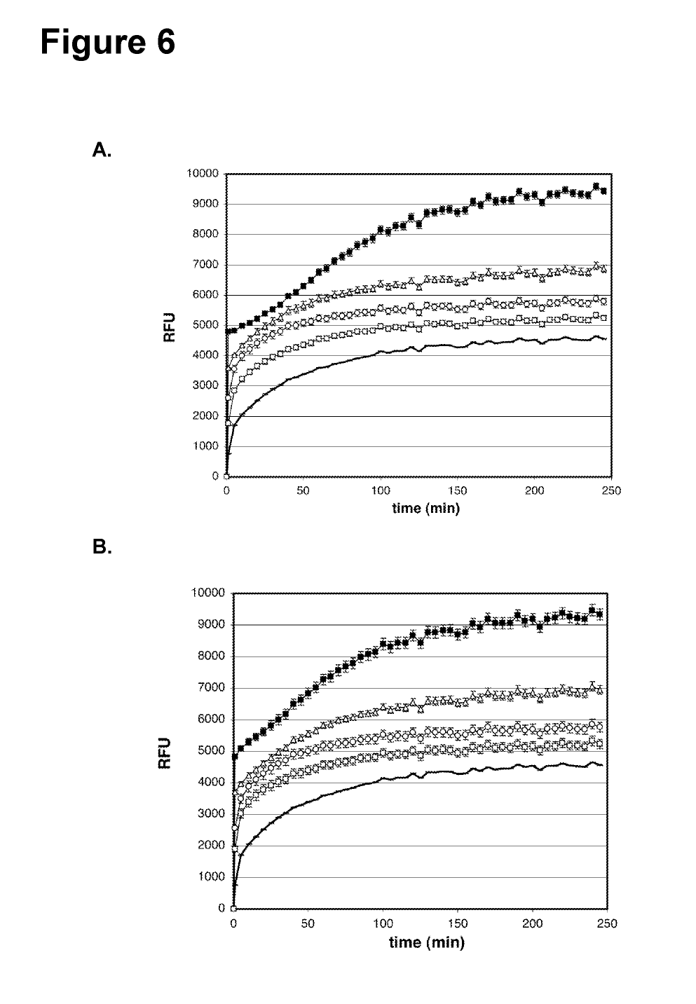

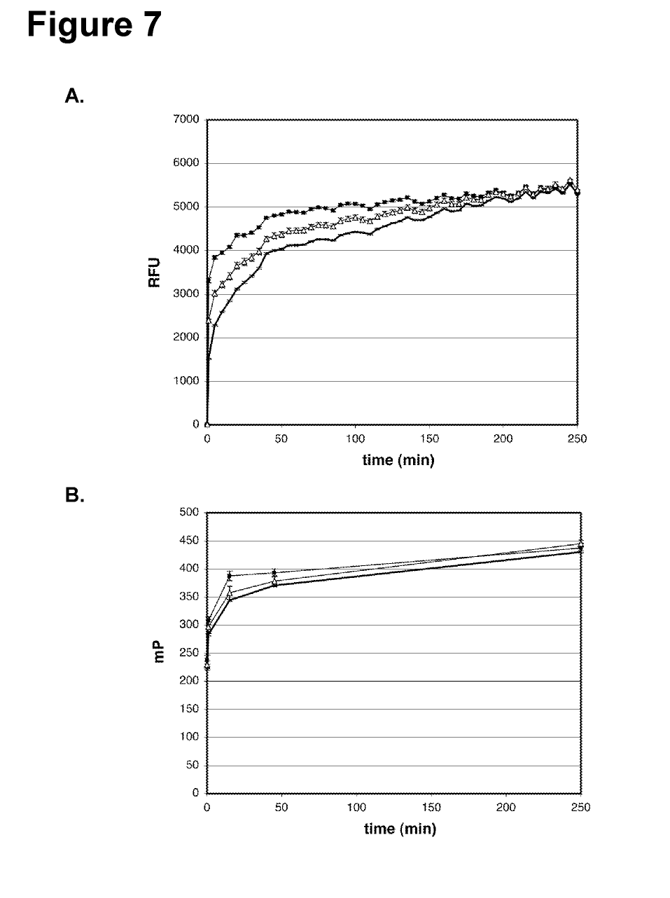

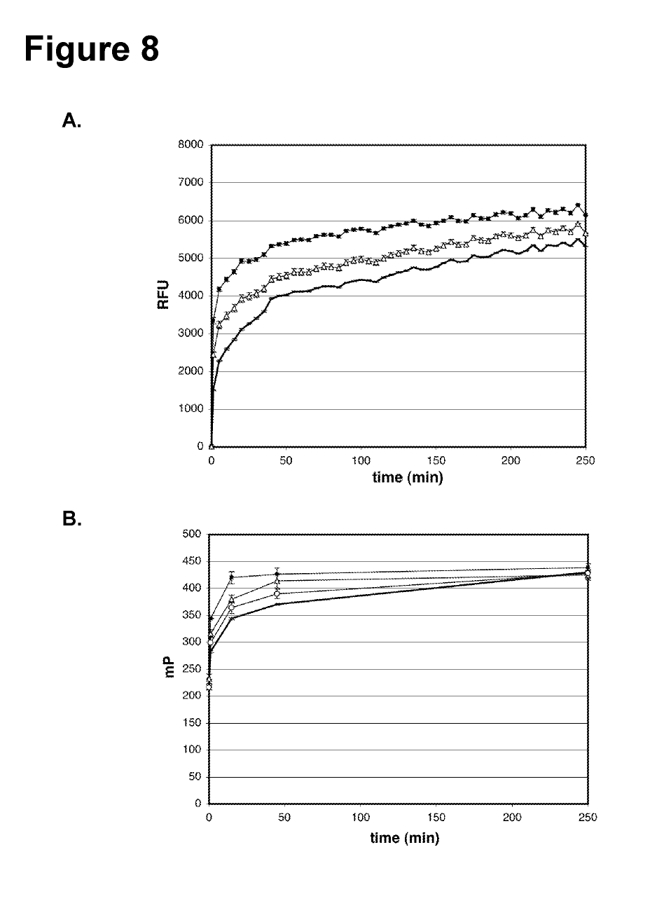

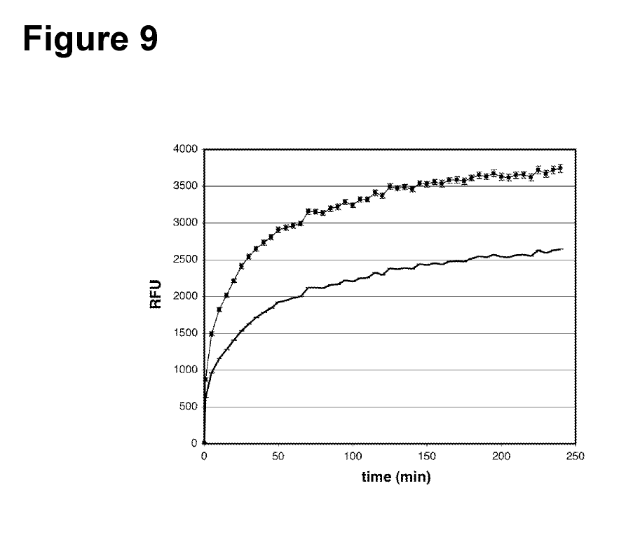

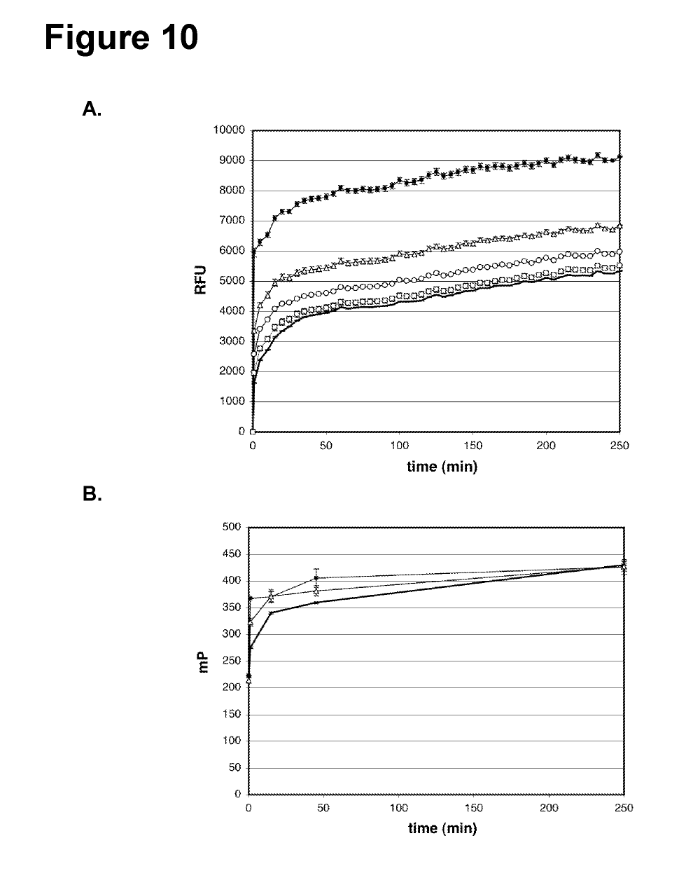

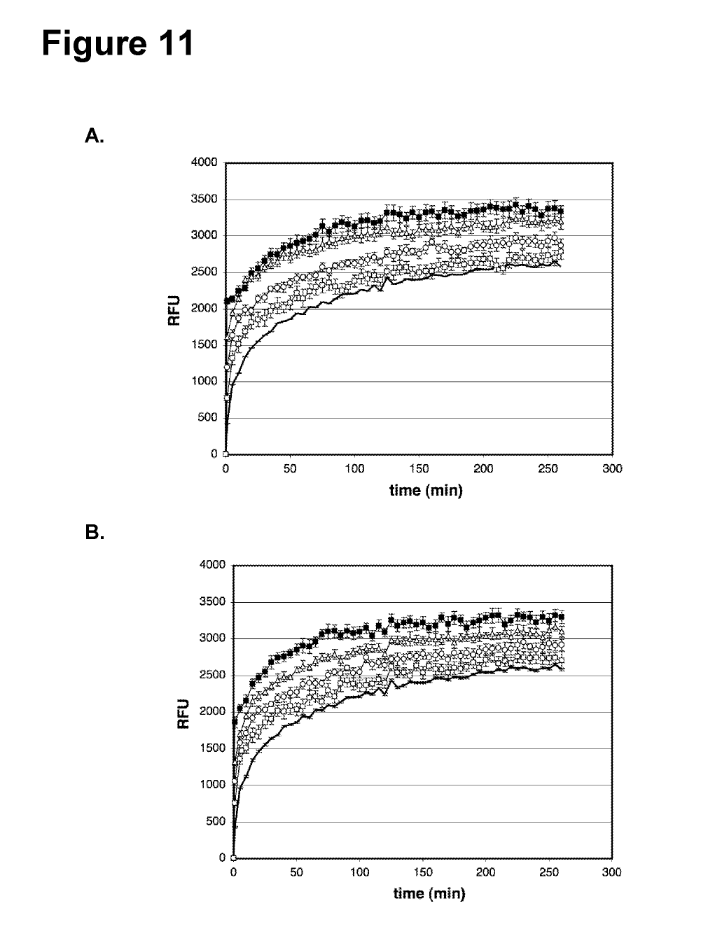

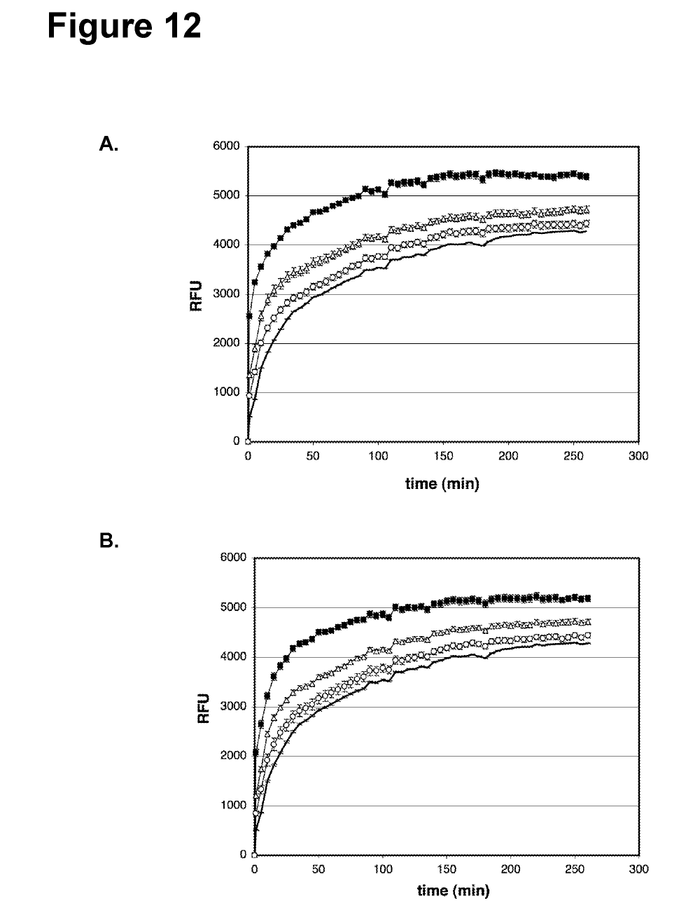

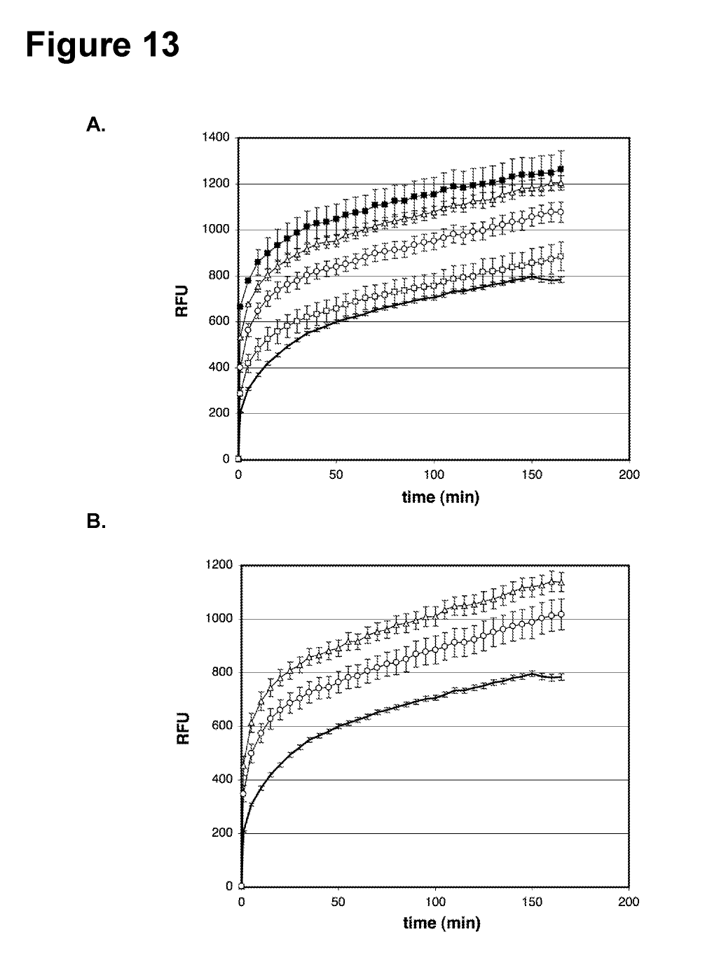

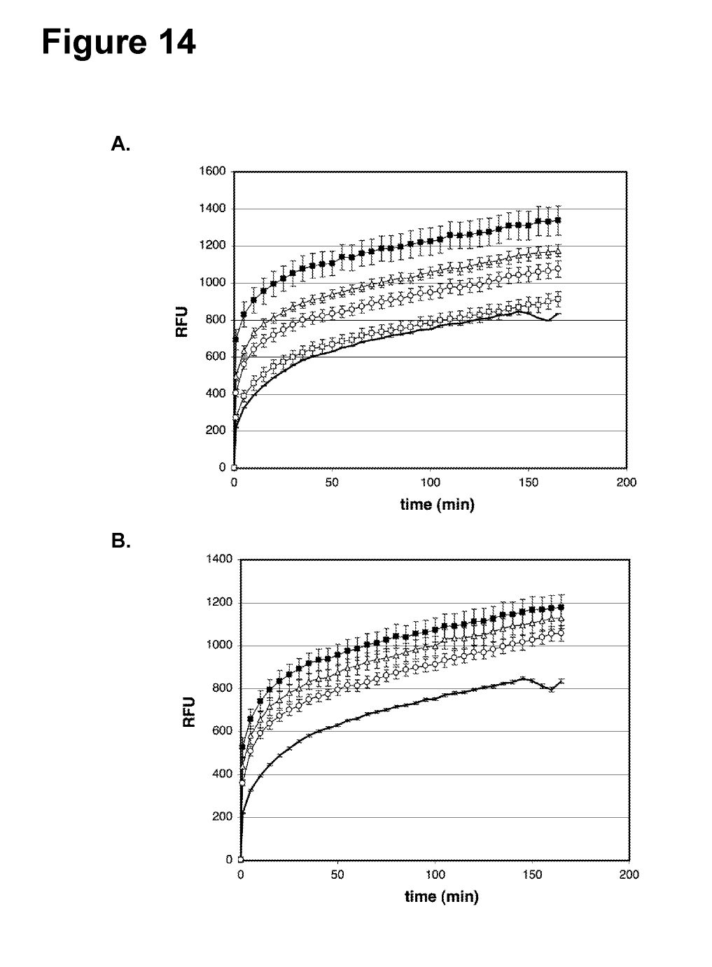

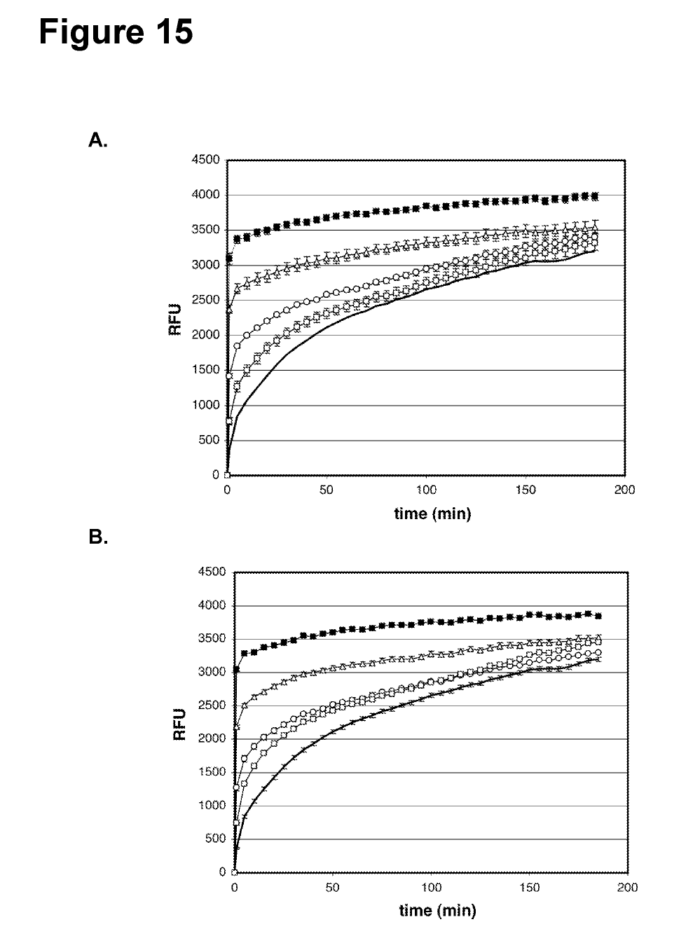

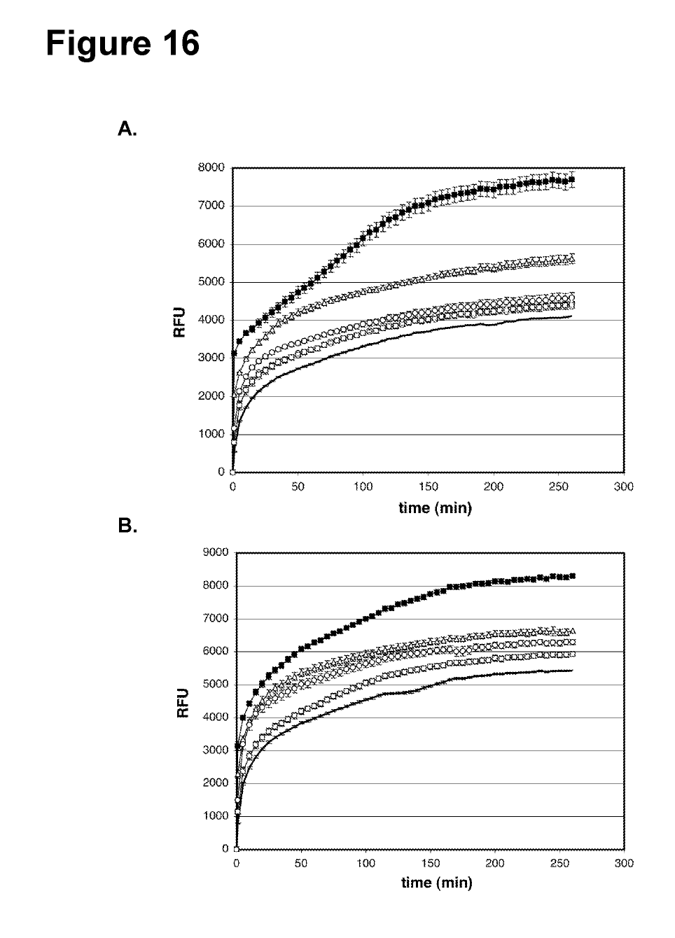

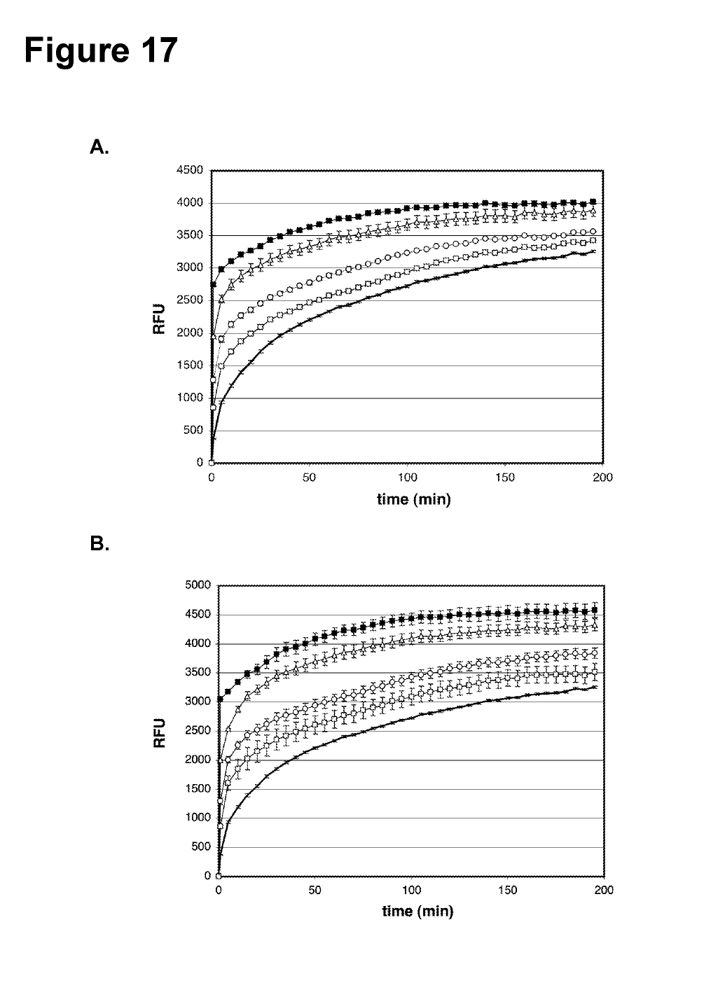

Indatraline (INDAT), LNK-121 (cis and trans), LNK-122, LNK-123 (cis and trans), LNK-124, LNK-125, LNK-126, LNK-130, LNK-1110 (cis and trans), LNK-1111 (cis and trans), LNK-1114 (cis and trans), LNK-1115 (cis and trans), LNK-1521 (cis and trans), and LNK-1800 (cis and trans) were found to bind to α-synuclein and affect the rate of protein aggregation in the presence of 1,1,1,3,3,3-hexafluoro-2-propanol (HFIP). Results are shown in FIGS. 1-17. HFIP induces rapid structure formation and aggregation of α-synuclein (Munishkina et al., Biochemistry 42:2720-30, 2003; Maiti, Apetri et al., J Am Chem Soc, 126:2399-408, 2004; each of which is incorporated herein by reference). Compounds were incubated with purified recombinant human α-synuclein (20 μM) in a buffer containing HFIP (25 mM Tris, pH 8.0, 3.1% HFIP) at room temperature. Structure formation was monitored by Thioflavin T fluorescence and fluorescence polarization. Thioflavin T fluorescence can be used to measure aggregation of amyloidogenic proteins, including α-synuclein (excitation, 440 nm; emission, 495 nm) (Naiki et al., Anal. Biochem., 177:244-9, 1989; Conway et al., Biochemistry, 39:2552-63, 2000; each of which is incorporated herein by reference). Average molecular size was monitored by fluorescence polarization using human α-synuclein covalently conjugated to Alexa Fluor 594 (excitation, 546 nm; emission, 620 nm). Assays were performed in a 384-well plate and readings taken directly from each well over time.

### Example 11

### Indatraline and Other Inventive Compounds Bind to α-Synuclein and Affect the Rate of Structure Formation in Aqueous Solution

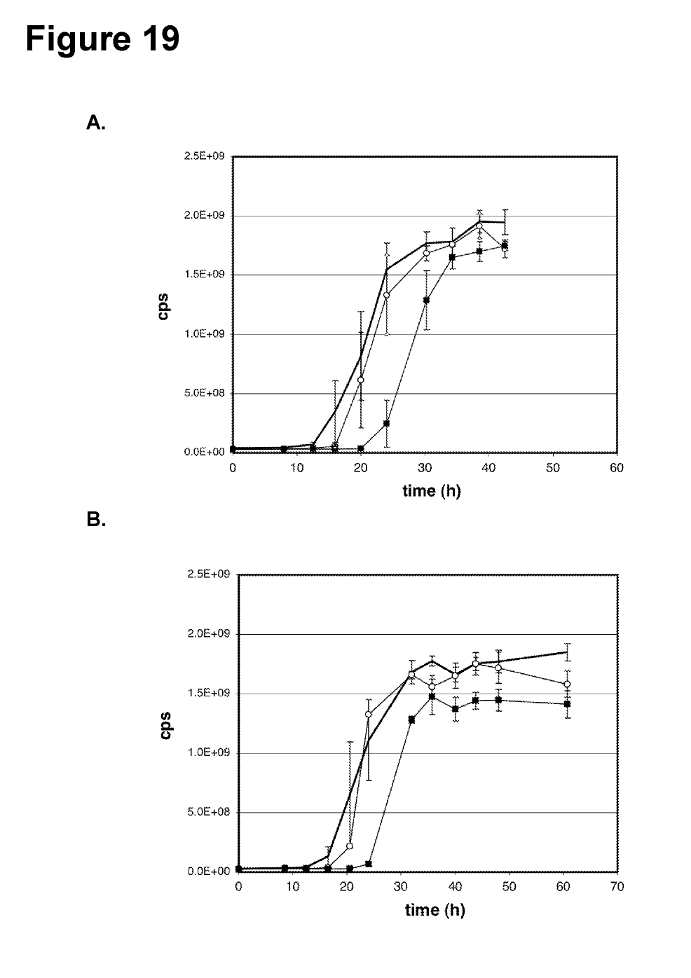

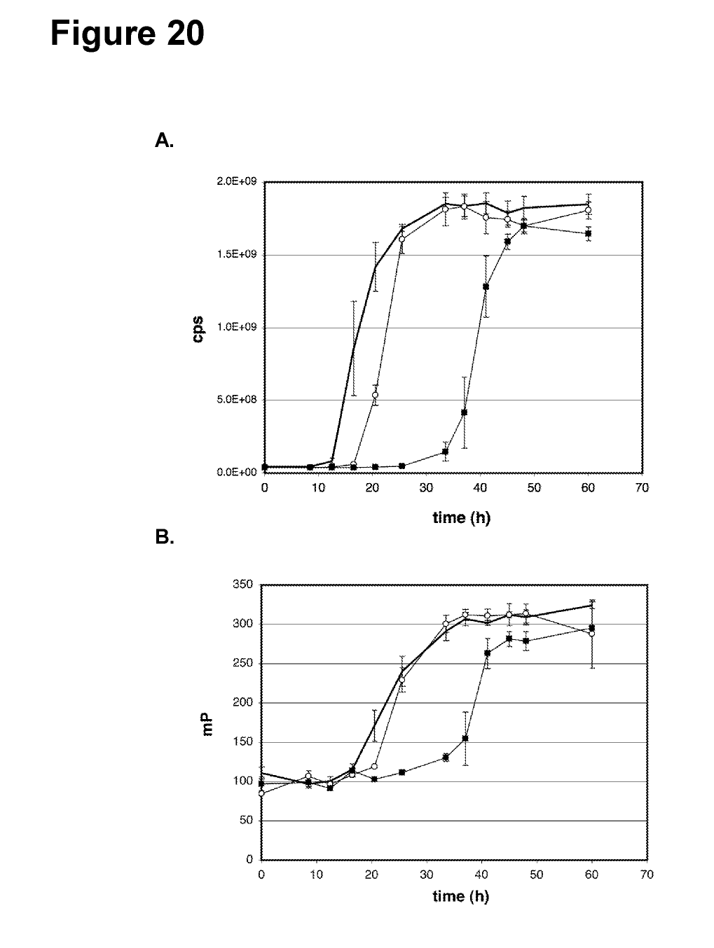

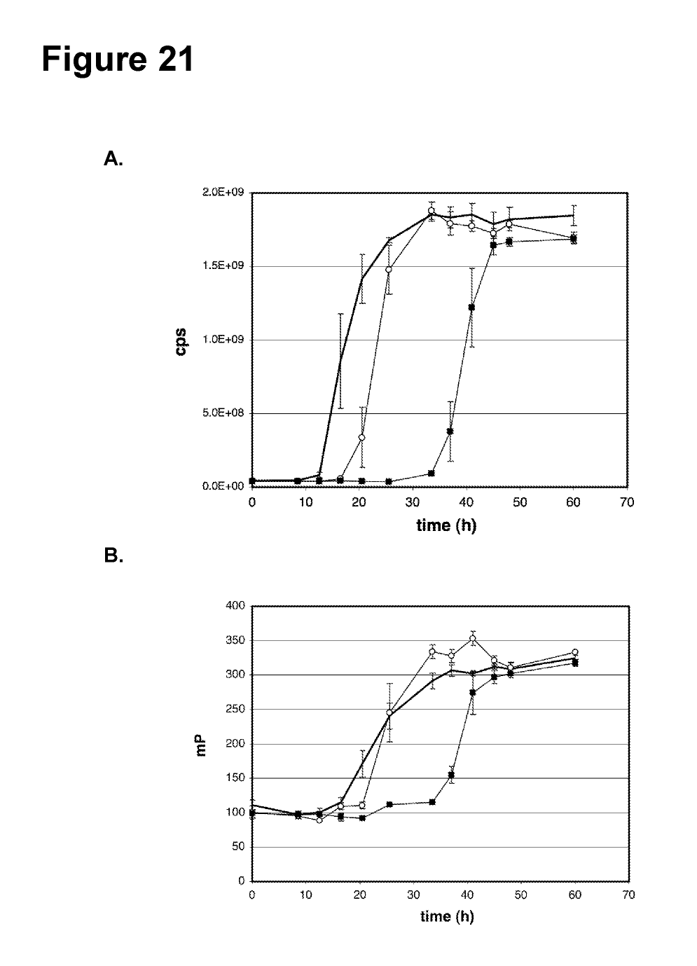

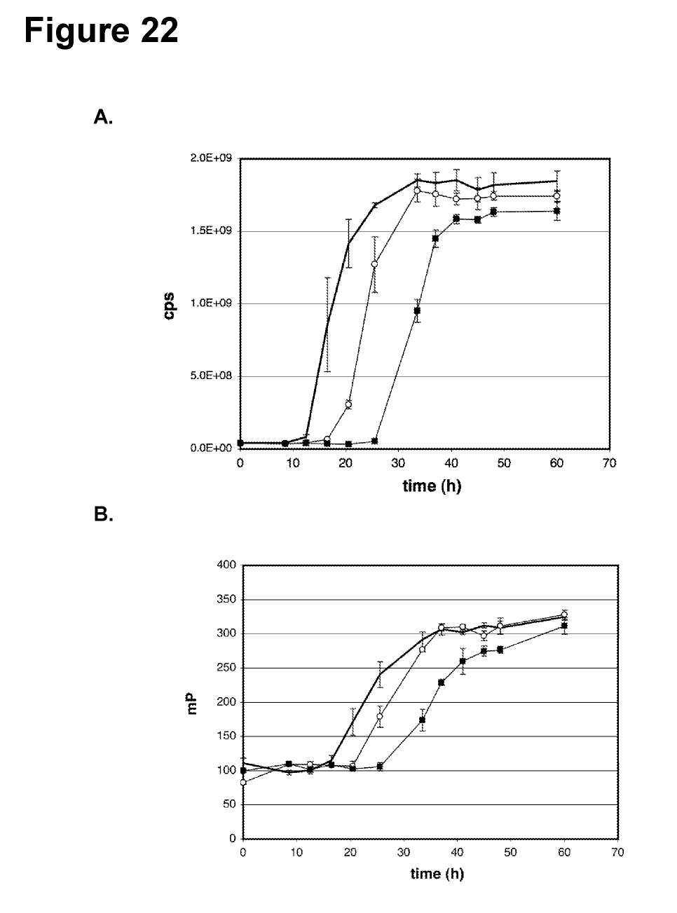

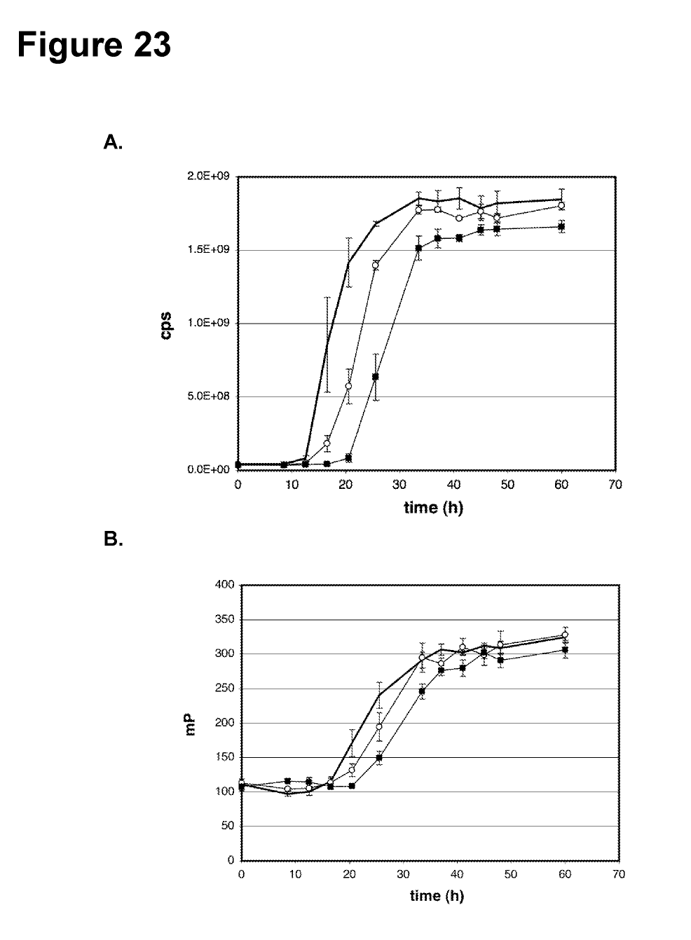

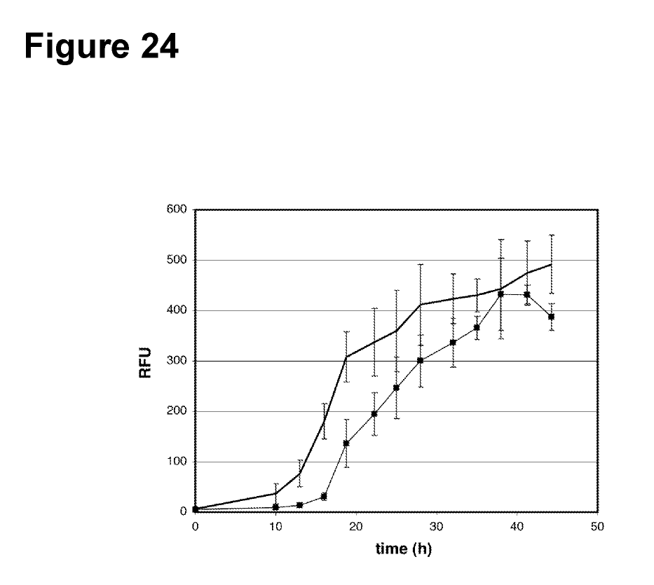

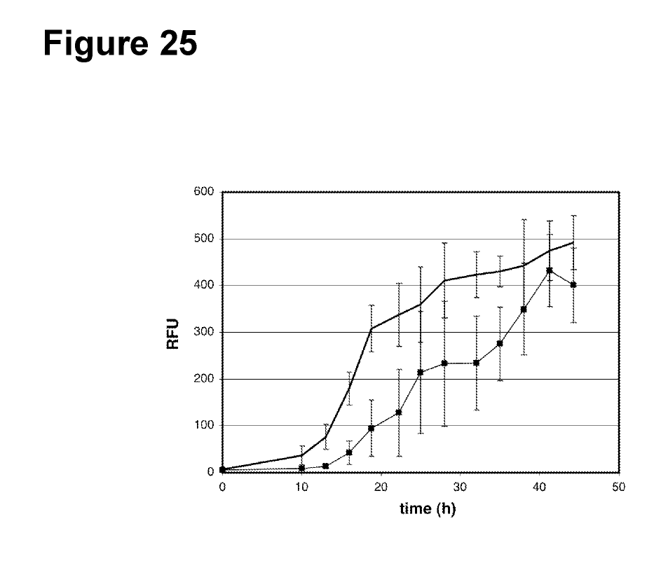

Indatraline, LNK-121, LNK-123, LNK-1111, LNK-1115, and LNK-1800 delay the initiation of α-synuclein aggregation in aqueous solution. Compounds were incubated with purified recombinant human α-synuclein (70 μM) in an aqueous buffer system (20 mM Bis-tris propane, pH 7.4, 100 mM LiCl) at 37° C. with gentle agitation. Structure formation was monitored by Thioflavin T fluorescence and/or fluorescence polarization. Thioflavin T fluorescence can be used to measure aggregation of amyloidogenic proteins, including α-synuclein (excitation, 440 nm; emission, 495 nm) (Naiki, H.; Higuchi, K.; Hosokawa, M.; Takeda, T., Fluorometric determination of amyloid fibrils in vitro using the fluorescent dye, thioflavin T1. Anal Biochem 1989, 177, (2), 244-9; incorporated herein by reference). Average molecular size was monitored by fluorescence polarization using human α-synuclein covalently conjugated to Alexa Fluor 594 (excitation, 546 nm; emission, 620 nm) (Luk, K. C.; Hyde, E. G.; Trojanowski, J. Q.; Lee, V. M., Sensitive fluorescence polarization technique for rapid screening of alpha-synuclein oligomerization/fibrillization inhibitors. Biochemistry 2007, 46, (44), 12522-9; incorporated herein by reference). See FIGS. 18-25.

### Example 12

### Drosophila Retinal Assay

Indatraline suppresses α-synuclein-induced retinal degeneration in transgenic Drosophila. Human A30P α-synuclein expression was directed to the retina with the glass multimer reporter (GMR) promoter. The transgene induces progressive retinal degeneration. Dry food (Formula 4-24; Carolina Biological Supply) was rehydrated with water or an aqueous solution of drug. Drug concentration refers to the final concentration of compound in the aqueous solution. Transgenic female flies less than 12-hours-old were placed on either untreated or drug-embedded food. For analysis of retinal degeneration, fly heads were annealed to a glass microscope, then eyes were transilluminated following optical neutralization of the corneas with immersion oil and examined under a 100× lens (Franceschini, N., Pupil and pseudopupil in the compound eye of Drosophila. In Information processing in the visual system of Drosophila, Wehner, R., Ed. Springer: Berlin, 1972; pp 75-82; incorporated here in by reference). For quantitation, 20-50 ommatidia per eye of at least 12 eyes were examined per group. The percent normal ommatidia was calculated for each eye and averaged for each sample set. Indatraline suppressed α-synuclein-induced retinal degeneration (FIG. 26A).

### Example 13

### Primary Neuronal Toxicity Assay

Indatraline decreased α-synuclein neurotoxicity in dopaminergic neurons. Midbrain cultures will be prepared from E17 rat ventral mesencephalon as described in a published protocol (Xu, J.; Kao, S. Y.; Lee, F. J.; Song, W.; Jin, L. W.; Yankner, B. A., Dopamine-dependent neurotoxicity of alpha-synuclein: a mechanism for selective neurodegeneration in Parkinson disease. Nat Med 2002, 8, (6), 600-6; incorporated herein by reference). Cultured cells were infected with a recombinant lentivirus encoding human A53T α-synuclein (A53T) or a control virus (none). Cells were treated with various concentrations of indatraline (black bars) for 3 days. Cells were then fixed and immunostained for Microtubule-associated protein 2, which stains all neurons, and Tyrosine Hydroxylase, a marker for dopaminergic neurons. Toxicity of A53T α-synuclein toward dopaminergic neurons was determined by calculating the percentage neurons positive for tyrosine hydroxylase (TH⁺ cells). Indatraline diminished toxicity of A53T α-synuclein toward dopaminergic neurons in a dose-dependent manner (FIG. 26B).

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A compound of formula: 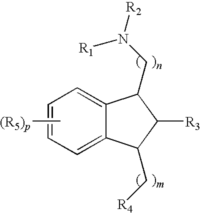
wherein
n is an integer between 0 and 6, inclusive;
m is an integer between 0 and 6, inclusive;
provided that both n and m are not 0;
p is an integer between 0 and 4, inclusive;
R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{A}; —CO₂R_{A}; —C(═O)N(R_{A})₂; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; —C(═O)N(R_{B})₂; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is substituted or unsubstituted, branched or unbranched aryl; or substituted or unsubstituted, branched or unbranched heteroaryl;
R₅ is halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; or a pharmaceutically acceptable salt, derivative, analog, isomer, or solvate thereof.

2.-4. (canceled)

5. The compound of claim 1, wherein n is 0 or 1.

6. (canceled)

7. The compound of claim 1, wherein m is 0 or 1.

8.-10. (canceled)

11. The compound of claim 1, wherein p is 0.

12.-13. (canceled)

14. The compound of claim 1, wherein at least one of R₁ and R₂ is hydrogen.

15. The compound of claim 1, wherein at least one of R₁ and R₂ is C₁-C₆ alkyl.

16.-21. (canceled)

22. The compound of claim 1, wherein R₁ is hydrogen, and R₂ is benzyl.

23. The compound of claim 1, wherein R₄ is substituted or unsubstituted aryl.

24.-25. (canceled)

26. The compound of claim 1, wherein R₄ is substituted phenyl.

27.-50. (canceled)

51. A compound of formula: 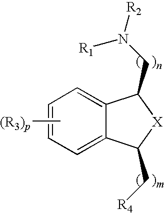
wherein
n is an integer between 0 and 6, inclusive;
m is an integer between 0 and 6, inclusive;
p is an integer between 0 and 4, inclusive;
R₁ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{A}; —CO₂R_{A}; —C(═O)N(R_{A})₂; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{B}; —CO₂R_{B}; —C(═O)N(R_{B})₂; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is substituted or unsubstituted, branched or unbranched aryl; or substituted or unsubstituted, branched or unbranched heteroaryl;
X is O, S, NH, or NR₆; wherein R₆ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{A}; —CO₂R_{A}; —C(═O)N(R_{A})₂; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable forms thereof.

52. A method of preventing aggregation of α-synuclein, the method comprising the contacting of α-synuclein protein with an effective amount of the compound of claim 1 or claim 51 to reduce the aggregation of α-synuclein.

53.-57. (canceled)

58. A method of treating a synculeinopathic subject, the method comprising administering to a synculeinopathic subject a therapeutically effective amount of compound of claim 1 or claim 51 or a pharmaceutically acceptable form thereof.

59. The method of claim 58, wherein the synucleinopathic subject has a synucleinopathy selected from the group consisting of Parkinson's disease, diffuse Lewy body disease, multiple system atrophy disorder, and pantothenate kinase-associated neurodegeneration.

60.-63. (canceled)

64. A method of treating a synucleinopathic subject, the method comprising administering to a synucleinopathic subject:
(a) a therapeutically effective amount of a farnesyl transferase inhibitor or a pharmaceutically acceptable form thereof and
(b) a therapeutically effective amount of a compound of claim 1 or claim 51 that inhibits the aggregation of α-synuclein or a pharmaceutically acceptable form thereof.

65. A pharmaceutical composition comprising a compound of claim 1 or claim 51 and a pharmaceutically acceptable excipient.

66. The pharmaceutical composition of claim 65 further comprising a farnesyl transferase inhibitor.

67.-72. (canceled)

73. A kit comprising a pharmaceutical composition of claim 65, and instructions for administration.

74. The compound of claim 1 selected from 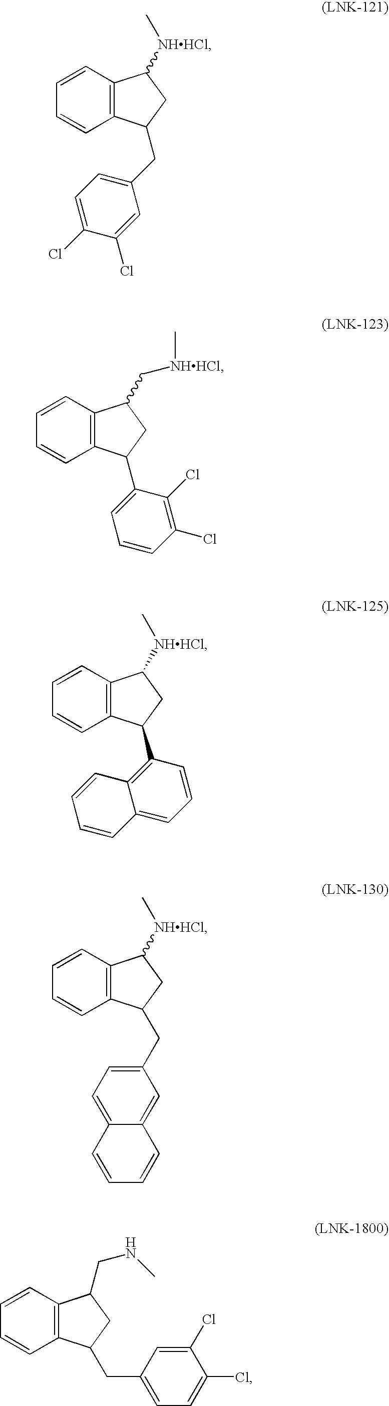 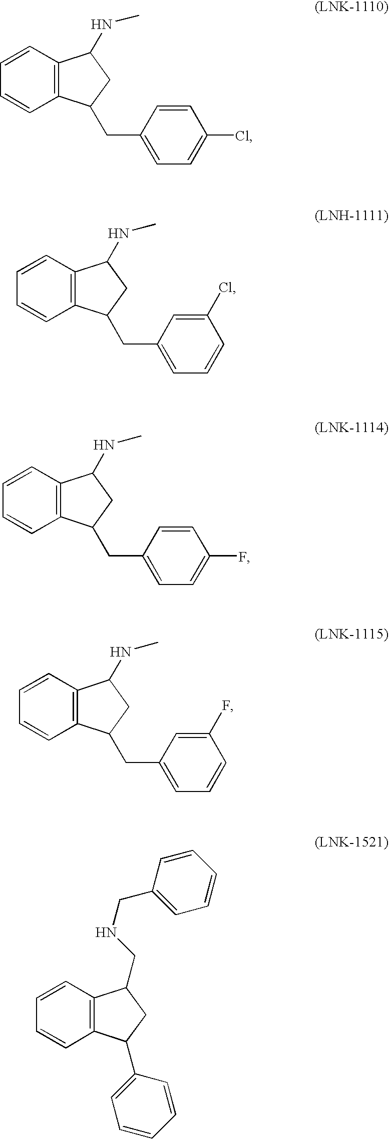
or a pharmaceutically acceptable salt thereof.
